SEQUENCE LISTING (16 sequences)

SEQ ID NO: 1 - protein, 2273 aa, Artificial Sequence [sequence omitted]

SEQ ID NO: 2 - DNA, 6822 bp, Artificial Sequence
  Synthetic Sequence
atgggcttcgtgagacagatacagcttttgctctggaagaactggaccctgcggaaaaggcaaaagattcgctttgtggtggaactcgtgtggcctttatctttatttctggtcttgatctggttaaggaatgccaacccactctacagccatcatgaatgccatttccccaacaaggcgatgccctcagcaggaatgctgccgtggctccaggggatcttctgcaatgtgaacaatccctgttttcaaagccccaccccaggagaatctcctggaattgtgtcaaactataacaactccatcttggcaagggtatatcgagattttcaagaactcctcatgaatgcaccagagagccagcaccttggccgtatttggacagagctacacatcttgtcccaattcatggacaccctccggactcacccggagagaattgcaggaagaggaatacgaataagggatatcttgaaagatgaagaaacactgacactatttctcattaaaaacatcggcctgtctgactcagtggtctaccttctgatcaactctcaagtccgtccagagcagttcgctcatggagtcccggacctggcgctgaaggacatcgcctgcagcgaggccctcctggagcgcttcatcatcttcagccagagacgcggggcaaagacggtgcgctatgccctgtgctccctctcccagggcaccctacagtggatagaagacactctgtatgccaacgtggacttcttcaagctcttccgtgtgcttcccacactcctagacagccgttctcaaggtatcaatctgagatcttggggaggaatattatctgatatgtcaccaagaattcaagagtttatccatcggccgagtatgcaggacttgctgtgggtgaccaggcccctcatgcagaatggtggtccagagacctttacaaagctgatgggcatcctgtctgacctcctgtgtggctaccccgagggaggtggctctcgggtgctctccttcaactggtatgaagacaataactataaggcctttctggggattgactccacaaggaaggatcctatctattcttatgacagaagaacaacatccttttgtaatgcattgatccagagcctggagtcaaatcctttaaccaaaatcgcttggagggcggcaaagcctttgctgatgggaaaaatcctgtacactcctgattcacctgcagcacgaaggatactgaagaatgccaactcaacttttgaagaactggaacacgttaggaagttggtcaaagcctgggaagaagtagggccccagatctggtacttctttgacaacagcacacagatgaacatgatcagagataccctggggaacccaacagtaaaagactttttgaataggcagcttggtgaagaaggtattactgctgaagccatcctaaacttcctctacaagggccctcgggaaagccaggctgacgacatggccaacttcgactggagggacatatttaacatcactgatcgcaccctccgcctggtcaatcaatacctggagtgcttggtcctggataagtttgaaagctacaatgatgaaactcagctcacccaacgtgccctctctctactggaggaaaacatgttctgggccggagtggtattccctgacatgtatccctggaccagctctctaccaccccacgtgaagtataagatccgaatggacatagacgtggtggagaaaaccaataagattaaagacaggtattgggattctggtcccagagctgatcccgtggaagatttccggtacatctggggcgggtttgcctatctgcaggacatggttgaacaggggatcacaaggagccaggtgcaggcggaggctccagttggaatctacctccagcagatgccctacccctgcttcgtggacgattctttcatgatcatcctgaaccgctgtttccctatcttcatggtgctggcatggatctactctgtctccatgactgtgaagagcatcgtcttggagaaggagttgcgactgaaggagaccttgaaaaatcagggtgtctccaatgcagtgatttggtgtacctggttcctggacagcttctccatcatgtcgatgagcatcttcctcctgacgatattcatcatgcatggaagaatcctacattacagcgacccattcatcctcttcctgttcttgttggctttctccactgccaccatcatgctgtgctttctgctcagcaccttcttctccaaggccagtctggcagcagcctgtagtggtgtcatctatttcaccctctacctgccacacatcctgtgcttcgcctggcaggaccgcatgaccgctgagctgaagaaggctgtgagcttactgtctccggtggcatttggatttggcactgagtacctggttcgctttgaagagcaaggcctggggctgcagtggagcaacatcgggaacagtcccacggaaggggacgaattcagcttcctgctgtccatgcagatgatgctccttgatgctgctgtctatggcttactcgcttggtaccttgatcaggtgtttccaggagactatggaaccccacttccttggtactttcttctacaagagtcgtattggcttggcggtgaagggtgttcaaccagagaagaaagagccctggaaaagaccgagcccctaacagaggaaacggaggatccagagcacccagaaggaatacacgactccttctttgaacgtgagcatccagggtgggttcctggggtatgcgtgaagaatctggtaaagatttttgagccctgtggccggccagctgtggaccgtctgaacatcaccttctacgagaaccagatcaccgcattcctgggccacaatggagctgggaaaaccaccaccttgtccatcctgacgggtctgttgccaccaacctctgggactgtgctcgttgggggaagggacattgaaaccagcctggatgcagtccggcagagccttggcatgtgtccacagcacaacatcctgttccaccacctcacggtggctgagcacatgctgttctatgcccagctgaaaggaaagtcccaggaggaggcccagctggagatggaagccatgttggaggacacaggcctccaccacaagcggaatgaagaggctcaggacctatcaggtggcatgcagagaaagctgtcggttgccattgcctttgtgggagatgccaaggtggtgattctggacgaacccacctctggggtggacccttactcgagacgctcaatctgggatctgctcctgaagtatcgctcaggcagaaccatcatcatgtccactcaccacatggacgaggccgacctccttggggaccgcattgccatcattgcccagggaaggctctactgctcaggcaccccactcttcctgaagaactgctttggcacaggcttgtacttaaccttggtgcgcaagatgaaaaacatccagagccaaaggaaaggcagtgaggggacctgcagctgctcgtctaagggtttctccaccacgtgtccagcccacgtcgatgacctaactccagaacaagtcctggatggggatgtaaatgagctgatggatgtagttctccaccatgttccagaggcaaagctggtggagtgcattggtcaagaacttatcttccttcttccaaataagaacttcaagcacagagcatatgccagccttttcagagagctggaggagacgctggctgaccttggtctcagcagttttggaatttctgacactcccctggaagagatttttctgaaggtcacggaggattctgattcaggacctctgtttgcgggtggcgctcagcagaaaagagaaaacgtcaacccccgacacccctgcttgggtcccagagagaaggctggacagacaccccaggactccaatgtctgctccccaggggcgccggctgctcacccagagggccagcctcccccagagccagagtgcccaggcccgcagctcaacacggggacacagctggtcctccagcatgtgcaggcgctgctggtcaagagattccaacacaccatccgcagccacaaggacttcctggcgcagatcgtgctcccggctacctttgtgtttttggctctgatgctttctattgttatccctccttttggcgaataccccgctttgacccttcacccctggatatatgggcagcagtacaccttcttcagcatggatgaaccaggcagtgagcagttcacggtacttgcagacgtcctcctgaataagccaggctttggcaaccgctgcctgaaggaagggtggcttccggagtacccctgtggcaactcaacaccctggaagactccttctgtgtccccaaacatcacccagctgttccagaagcagaaatggacacaggtcaacccttcaccatcctgcaggtgcagcaccagggagaagctcaccatgctgccagagtgccccgagggtgccgggggcctcccgcccccccagagaacacagcgcagcacggaaattctacaagacctgacggacaggaacatctccgacttcttggtaaaaacgtatcctgctcttataagaagcagcttaaagagcaaattctgggtcaatgaacagaggtatggaggaatttccattggaggaaagctcccagtcgtccccatcacgggggaagcacttgttgggtttttaagcgaccttggccggatcatgaatgtgagcgggggccctatcactagagaggcctctaaagaaatacctgatttccttaaacatctagaaactgaagacaacattaaggtgtggtttaataacaaaggctggcatgccctggtcagctttctcaatgtggcccacaacgccatcttacgggccagcctgcctaaggacaggagccccgaggagtatggaatcaccgtcattagccaacccctgaacctgaccaaggagcagctctcagagattacagtgctgaccacttcagtggatgctgtggttgccatctgcgtgattttctccatgtccttcgtcccagccagctttgtcctttatttgatccaggagcgggtgaacaaatccaagcacctccagtttatcagtggagtgagccccaccacctactgggtgaccaacttcctctgggacatcatgaattattccgtgagtgctgggctggtggtgggcatcttcatcgggtttcagaagaaagcctacacttctccagaaaaccttcctgcccttgtggcactgctcctgctgtatggatgggcggtcattcccatgatgtacccagcatccttcctgtttgatgtccccagcacagcctatgtggctttatcttgtgctaatctgttcatcggcatcaacagcagtgctattaccttcatcttggaattatttgagaataaccggacgctgctcaggttcaacgccgtgctgaggaagctgctcattgtcttcccccacttctgcctgggccggggcctcattgaccttgcactgagccaggctgtgacagatgtctatgcccggtttggtgaggagcactctgcaaatccgttccactgggacctgattgggaagaacctgtttgccatggtggtggaaggggtggtgtacttcctcctgaccctgctggtccagcgccacttcttcctctcccaatggattgccgagcccactaaggagcccattgttgatgaagatgatgatgtggctgaagaaagacaaagaattattactggtggaaataaaactgacatcttaaggctacatgaactaaccaagatttatccaggcacctccagcccagcagtggacaggctgtgtgtcggagttcgccctggagagtgctttggcctcctgggagtgaatggtgccggcaaaacaaccacattcaagatgctcactggggacaccacagtgacctcaggggatgccaccgtagcaggcaagagtattttaaccaatatttctgaagtccatcaaaatatgggctactgtcctcagtttgatgcaattgatgagctgctcacaggacgagaacatctttacctttatgcccggcttcgaggtgtaccagcagaagaaatcgaaaaggttgcaaactggagtattaagagcctgggcctgactgtctacgccgactgcctggctggcacgtacagtgggggcaacaagcggaaactctccacagccatcgcactcattggctgcccaccgctggtgctgctggatgagcccaccacagggatggacccccaggcacgccgcatgctgtggaacgtcatcgtgagcatcatcagagaagggagggctgtggtcctcacatcccacagcatggaagaatgtgaggcactgtgtacccggctggccatcatggtaaagggcgcctttcgatgtatgggcaccattcagcatctcaagtccaaatttggagatggctatatcgtcacaatgaagatcaaatccccgaaggacgacctgcttcctgacctgaaccctgtggagcagttcttccaggggaacttcccaggcagtgtgcagagggagaggcactacaacatgctccagttccaggtctcctcctcctccctggcgaggatcttccagctcctcctctcccacaaggacagcctgctcatcgaggagtactcagtcacacagaccacactggaccaggtgtttgtaaattttgctaaacagcagactgaaagtcatgacctccctctgcaccctcgagctgctggagccagtcgacaagcccaggactga

SEQ ID NO: 3 - DNA, 1404 bp, Artificial Sequence
  Synthetic Sequence
gatccaacaaaagtgattataccccccaaaatatgatggtagtatcttatactaccatcattttataggcatagggctcttagctgcaaataatggaactaactctaataaagcagaacgcaaatattgtaaatattagagagctaacaatctctgggatggctaaaggatggagcttggaggctacccagccagtaacaatattccgggctccactgttgaatggagacactacaactgccttggatgggcagagatattatggatgctaagccccaggtgctaccattaggacttctaccactgtcctaacgggtggagcccatcacatgcctatgccctcactgtaaggaaatgaagctactgttgtatatcttgggaagcacttggattaattgttatacagttttgttgaagaagacccctagggtaagtagccataactgcacactaaatttaaaattgttaatgagtttctcaaaaaaaatgttaaggttgttagctggtatagtatatatcttgcctgttttccaaggacttctttgggcagtaccttgtctgtgctggcaagcaactgagacttaatgaaagagtattggagatatgaatgaattgatgctgtatactctcagagtgccaaacatataccaatggacaagaaggtgaggcagagagcagacaggcattagtgacaagcaaagatatgcagaatttcattctcagcaaatcaaaagtcctcaacctggttggaagaatattggcactgaatggtatcaataaggttgctagagagggttagaggtgcacaatgtgcttccataacattttatacttctccaatcttagcactaatcaaacatggttgaatactttgtttactataactcttacagagttataagatctgtgaagacagggacagggacaatacccatctctgtctggttcataggtggtatgtaatagatatttttaaaaataagtgagttaatgaatgagggtgagaatgaaggcacagaggtattagggggaggtgggccccagagaatggtgccaaggtccagtggggtgactgggatcagctcaggcctgacgctggccactcccacctagctcctttctttctaatctgttctcattctccttgggaaggattgaggtctctggaaaacagccaaacaactgttatgggaacagcaagcccaaataaagccaagcatcagggggatctgagagctgaaagcaacttctgttccccctccctcagctgaaggggtggggaagggctcccaaagccataactccttttaagggatttagaaggcataaaaaggcccctggctgagaacttccttcttcattctgcagttggtgccagaactctggatcctgaactggaagaaa

SEQ ID NO: 4 - DNA, 1325 bp, Artificial Sequence
  Synthetic Sequence
gctgcctactgaggcacacaggggcgcctgcctgctgcccgctcagccaaggcggtgttgctggagccagcttgggacagctctcccaacgctctgccctggccttgcgacccactctctgggccgtagttgtctgtctgttaagtgaggaaagtgcccatctccagaggcattcagcggcaaagcagggcttccaggttccgaccccatagcaggacttcttggatttctacagccagtcagttgcaagcagcacccatattatttctataagaagtggcaggagctggatctgaagagtcagcagtctacctttccctgtttcttgtgctttatgcagtcaggaggaatgatctggattccatgtgaagcctgggaccacggagacccaagacttcctgcttgattctccctgcgaactgcaggctgtgggctgagccttcaagaagcaggagtcccctctagccattaactctcagagctaacctcatttgaatgggaacactagtcctgtgatgtctggaaggtgggcgcctctacactccacaccctacatggtggtccagacacatcattcccagcattagaaagctctagggggacccgttctgttccctgaggcattaaagggacatagaaataaatctcaagctctgaggctgatgccagcctcagactcagcctctgcactgtatgggccaattgtagccccaaggacttcttcttgctgcaccccctatctgtccacacctaaaacgatgggcttctattagttacagaactctctggcctgttttgttttgctttgctttgttttgttttgtttttttgtttttttgttttttagctatgaaacagaggtaatatctaatacagataacttaccagtaatgagtgcttcctacttactgggtactgggaagaagtgctttacacatattttctcatttaatctacacaataagtaattaagacatttccctgaggccacgggagagacagtggcagaacagttctccaaggaggacttgcaagttaataactggactttgcaaggctctggtggaaactgtcagcttgtaaaggatggagcacagtgtctggcatgtagcaggaactaaaataatggcagtgattaatgttatgatatgcagacacaacacagcaagataagatgcaatgtaccttctgggtcaaaccaccctggccactcctccccgatacccagggttgatgtgcttgaattagacaggattaaaggcttactggagctggaagccttgccccaactcaggagtttagccccagaccttctgtccaccagc

SEQ ID NO: 5 - DNA, 624 bp, Artificial Sequence
  Synthetic Sequence
aattctgtcattttactagggtgatgaaattcccaagcaacaccatccttttcagataagggcactgaggctgagagaggagctgaaacctacccggggtcaccacacacaggtggcaaggctgggaccagaaaccaggactgttgactgcagcccggtattcattctttccatagcccacagggctgtcaaagaccccagggcctagtcagaggctcctccttcctggagagttcctggcacagaagttgaagctcagcacagccccctaacccccaactctctctgcaaggcctcaggggtcagaacactggtggagcagatcctttagcctctggattttagggccatggtagagggggtgttgccctaaattccagccctggtctcagcccaacaccctccaagaagaaattagaggggccatggccaggctgtgctagccgttgcttctgagcagattacaagaagggactaagacaaggactcctttgtggaggtcctggcttagggagtcaagtgacggcggctcagcactcacgtgggcagtgccagcctctaagagtgggcaggggcactggccacagagtcccagggagtcccaccagcctagtcgccagacct

SEQ ID NO: 6 - DNA, 200 bp, Artificial Sequence
  Synthetic Sequence
acgcctgcaacaggcaggagatcccccaacagttactcccagccttcattccacagggtctggttttcctggaggtgggaagtcccagggtctgaggagagggagcgcaggcccccatttgtaggagtgagtcagctgacccgcccccggggttcctaatctcactaagaaagactttgctgatgacagggtttcctggg

SEQ ID NO: 7 - DNA, 295 bp, Artificial Sequence
  Synthetic Sequence
gggccccagaagcctggtggttgtttgtccttctcaggggaaaagtgaggcggccccttggaggaaggggccgggcagaatgatctaatcggattccaagcagctcaggggattgtctttttctagcaccttcttgccactcctaagcgtcctccgtgaccccggctgggatttagcctggtgctgtgtcagccccggtctcccaggggcttcccagtggtccccaggaaccctcgacagggccagggcgtctctctcgtccagcaagggcagggacgggccacaggccaagggc

SEQ ID NO: 8 - DNA, 2026 bp, Artificial Sequence
  Synthetic Sequence
ctggtgattacattagggcccacctggataatccagaatgatctccctatttcaacatccttaatttattcacatctgcaaagtctctttttcatataaggtaatgttcatcggttcccaggattaagacctgacatctttgggggcataattcagcttgccacagtaggtaaaaattcattgagctgcagttaagatttgtgaattttacctcagtcaagaaatgcacaaacttctggaaaagagtaatgatttacattccatcataataatgaattaaagacctagcagatctactcttttcctaccgagaggcccatggatctgagtagaaagagaagataagcgggattgagtacctaaaagggaggtaggagcctcgagtgtgggtctaaagacaaaaacaggctgaccactagtcattctagagatctgggaaaggtttcctgaatgatgaaaataagcatacaagaagagaggccttcctttcctgccattgaatattgccatgtctggcatgaaaagtagattcattctgacttttcgccttcctcgcagacaccaaccttggcatgtatacaaatctttcctgtatgtccagcatcagttcctatcccactgtggtacctgcagaatctgggcttcttgcactatctgaaagcccctgagaaggagagagttatagtaactaaacaaccaggccctgagatgcatattggctaggaatggcaggggctgacactgtgaactgtgcaaagagaatatgggacagctgtccagggccctcagtgaggggcaggagttagggaaggccctgcccagccctctgagccatagccatagccatcctctgaggaatggacaccccattgtgggggttggggttgagggctgtgtctatagataactactaatgtccagactgctgtaaggggaggtgaaggaggtcagagtcctgaaaccccagagcttatagattctgtctctacattttctatgcccgtgaagcctgagcctaggccctgtgggaaggacagtcaagaaaggaagattactttgttgttgctgttgtgggggtcctggcagctgaagagacagaaatatctctaattccatgagcggtcatacgaggcaagagaagctgcttagagcatggacttagttagtttcagggattggacagagtcaagagctggggtgaggaggtttaccctcggtaggggtgacacagatgtcaaccgcctattccctccacatgcatgtcctgccagaagaacctgtccctgggctgggaatcttatattaccttcctctccaatgagaagagaagttcaaggctcacagacatgtgcatacacagctcaatgcactcagatccccctccaccactcctgcccccactacctacaggagattgactcctgctgtgcacataagctgggataatcagggtttctaaacatcagcttcaaaagtccaatgtcccaaagtggtggggggctggggacgaggtactctttcccatacccttggcttttgtgtggcctggagccgctgatatagagattggagtgggacacgaggtattcctttcaaaaacacaaaggcctatactttgagccctcccatttcaatcccccaccatgcttcacctttaagacctccaactccactttgatcccagttctcaggttcaaggcctcacaaggccaaaatcctgaagttacccttctcaaactcccttgcctttaacatcatcagaatcaacctcctacccccactctgtcccagcagcaatagcctgctaatcttttagccactaatcttttaggcactaatctgctttccaaactcttggcacctgaactatttatagcagtgttttatgcccccccaccaagaaccctattcttttcccatgacccccaccaatcaaaacactcagaggactgtgggtataagaggctggggaggcaggcatagcaaccagagctggagactgatgtgaacttcatctctctcccca

SEQ ID NO: 9 - DNA, 1726 bp, Artificial Sequence
  Synthetic Sequence
gaggctgaggggtggggaaagggcatgggtgtttcatgaggacagagcttccgtttcatgcaatgaaaagagtttggagacggatggtggtgactggactatacacttacacacggtagcgatggtacactttgtattatgtatattttaccacgatctttttaaagtgtcaaaggcaaatggccaaatggttccttgtcctatagctgtagcagccatcggctgttagtgacaaagcccctgagtcaagatgacagcagcccccataactcctaatcggctctcccgcgtggagtcatttaggagtagtcgcattagagacaagtccaacatctaatcttccaccctggccagggccccagctggcagcgagggtgggagactccgggcagagcagagggcgctgacattggggcccggcctggcttgggtccctctggcctttccccaggggccctctttccttggggctttcttgggccgccactgctcccgctcctctccccccatcccaccccctcaccccctcgttcttcatatccttctctagtgctccctccactttcatccacccttctgcaagagtgtgggaccacaaatgagttttcacctggcctggggacacacgtgcccccacaggtgctgagtgactttctaggacagtaatctgctttaggctaaaatgggacttgatcttctgttagccctaatcatcaattagcagagccggtgaaggtgcagaacctaccgcctttccaggcctcctcccacctctgccacctccactctccttcctgggatgtgggggctggcacacgtgtggcccagggcattggtgggattgcactgagctgggtcattagcgtaatcctggacaagggcagacagggcgagcggagggccagctccggggctcaggcaaggctgggggcttcccccagacaccccactcctcctctgctggacccccacttcatagggcacttcgtgttctcaaagggcttccaaatagcatggtggccttggatgcccagggaagcctcagagttgcttatctccctctagacagaaggggaatctcggtcaagagggagaggtcgccctgttcaaggccacccagccagctcatggcggtaatgggacaaggctggccagccatcccaccctcagaagggacccggtggggcaggtgatctcagaggaggctcacttctgggtctcacattcttggatccggttccaggcctcggccctaaatagtctccctgggctttcaagagaaccacatgagaaaggaggattcgggctctgagcagtttcaccacccaccccccagtctgcaaatcctgacccgtgggtccacctgccccaaaggcggacgcaggacagtagaagggaacagagaacacataaacacagagagggccacagcggctcccacagtcaccgccaccttcctggcggggatgggtggggcgtctgagtttggttcccagcaaatccctctgagccgcccttgcgggctcgcctcaggagcaggggagcaagaggtgggaggaggaggtctaagtcccaggcccaattaagagatcaggtagtgtagggtttgggagcttttaaggtgaagaggcccgggctgatcccacaggccagtataaagcgccgtgaccctcaggtgatgcgccagggccggctgccgtcggggacagggctttccatagccagg

SEQ ID NO: 10 - protein, 572 aa, Artificial Sequence
  Synthetic Sequence
MAQHSDYSDDEFCADKLSNYSCDSDLENASTSDEDSSDDEVMVRPRTLRRRRISSSSSDSESDIEGGREEWSHVDNPPVLEDFLGHQGLNTDAVINNIEDAVKLFIGDDFFEFLVEESNRYYNQNRNNFKLSKKSLKWKDITPQEMKKFLGLIVLMGQVRKDRRDDYWTTEPWTETPYFGKTMTRDRFRQIWKAWHFNNNADIVNESDRLCKVRPVLDYFVPKFINIYKPHQQLSLDEGIVPWRGRLFFRVYNAGKIVKYGILVRLLCESDTGYICNMEIYCGEGKRLLETIQTVVSPYTDSWYHIYMDNYYNSVANCEALMKNKFRICGTIRKNRGIPKDFQTISLKKGETKFIRKNDILLQVWQSKKPVYLISSIHSAEMEESQNIDRTSKKKIVKPNALIDYNKHMKGVDRADQYLSYYSILRRTVKWTKRLAMYMINCALFNSYAVYKSVRQRKMGFKMFLKQTAIHWLTDDIPEDMDIVPDLQPVPSTSGMRAKPPTSDPPCRLSMDMRKHTLQAIVGSGKKKNILRRCRVCSVHKLRSETRYMCKFCNIPLHKGACFEKYHTLKNY

SEQ ID NO: 11 - DNA, 1719 bp, Artificial Sequence
  Synthetic Sequence
atggcccagcacagcgactacagcgacgacgagttctgtgccgataagctgagtaactacagctgcgacagcgacctggaaaacgccagcacatccgacgaggacagctctgacgacgaggtgatggtgcggcccagaaccctgagacggagaagaatcagcagctctagcagcgactctgaatccgacatcgagggcggccgggaagagtggagccacgtggacaaccctcctgttctggaagattttctgggccatcagggcctgaacaccgacgccgtgatcaacaacatcgaggatgccgtgaagctgttcataggagatgatttctttgagttcctggtcgaggaatccaaccgctattacaaccagaatagaaacaacttcaagctgagcaagaaaagcctgaagtggaaggacatcacccctcaggagatgaaaaagttcctgggactgatcgttctgatgggacaggtgcggaaggacagaagggatgattactggacaaccgaaccttggaccgagaccccttactttggcaagaccatgaccagagacagattcagacagatctggaaagcctggcacttcaacaacaatgctgatatcgtgaacgagtctgatagactgtgtaaagtgcggccagtgttggattacttcgtgcctaagttcatcaacatctataagcctcaccagcagctgagcctggatgaaggcatcgtgccctggcggggcagactgttcttcagagtgtacaatgctggcaagatcgtcaaatacggcatcctggtgcgccttctgtgcgagagcgatacaggctacatctgtaatatggaaatctactgcggcgagggcaaaagactgctggaaaccatccagaccgtcgtttccccttataccgacagctggtaccacatctacatggacaactactacaattctgtggccaactgcgaggccctgatgaagaacaagtttagaatctgcggcacaatcagaaaaaacagaggcatccctaaggacttccagaccatctctctgaagaagggcgaaaccaagttcatcagaaagaacgacatcctgctccaagtgtggcagtccaagaaacccgtgtacctgatcagcagcatccatagcgccgagatggaagaaagccagaacatcgacagaacaagcaagaagaagatcgtgaagcccaatgctctgatcgactacaacaagcacatgaaaggcgtggaccgggccgaccagtacctgtcttattactctatcctgagaagaacagtgaaatggaccaagagactggccatgtacatgatcaattgcgccctgttcaacagctacgccgtgtacaagtccgtgcgacaaagaaaaatgggattcaagatgttcctgaagcagacagccatccactggctgacagacgacattcctgaggacatggacattgtgccagatctgcaacctgtgcccagcacctctggtatgagagctaagcctcccaccagcgatcctccatgtagactgagcatggacatgcggaagcacaccctgcaggccatcgtcggcagcggcaagaagaagaacatccttagacggtgcagggtgtgcagcgtgcacaagctgcggagcgagactcggtacatgtgcaagttttgcaacattcccctgcacaagggagcctgcttcgagaagtaccacaccctgaagaattactag

SEQ ID NO: 12 - DNA, 1719 bp, Artificial Sequence
  Synthetic Sequence
atggcccagcacagcgactacagcgacgacgagttctgtgccgataagctgagtaactacagctgcgacagcgacctggaaaacgccagcacatccgacgaggacagctctgacgacgaggtgatggtgcggcccagaaccctgagacggagaagaatcagcagctctagcagcgactctgaatccgacatcgagggcggccgggaagagtggagccacgtggacaaccctcctgttctggaagattttctgggccatcagggcctgaacaccgacgccgtgatcaacaacatcgaggatgccgtgaagctgttcataggagatgatttctttgagttcctggtcgaggaatccaaccgctattacaaccagaagagaaacaacttcaagctgagcaagaaaagcctgaagtggaaggacatcacccctcaggagatgaaaaagttcctgggactgatcgttctgatgggacaggtgcggaaggacagaagggatgattactggacaaccgaaccttggaccgagaccccttactttggcaagaccatgaccagagacagattcagacagatctggaaagcctggcacttcaacaacaatgctgatatcgtgaacgagtctgatagactgtgtaaagtgcggccagtgttggattacttcgtgcctaagttcatcaacatctataagcctcaccagcagctgagcctggatgaaggcatcgtgccctggcggggcagactgttcttcagagtgtacaatgctggcaagatcgtcaaatacggcatcctggtgcgccttctgtgcgagagcgatacaggctacatctgtaatatggaaatctactgcggcgagggcaaaagactgctggaaaccatccagaccgtcgtttccccttataccgacagctggtaccacatctacatggacaactactacaattctgtggccaactgcgaggccctgatgaagaacaagtttagaatctgcggcacaatcagaaaaaacagaggcatccctaaggacttccagaccatctctctgaagaagggcgaaaccaagttcatcagaaagaacgacatcctgctccaagtgtggcagtccaagaaacccgtgtacctgatcagcagcatccatagcgccgagatggaagaaagccagaacatcgacagaacaagcaagaagaagatcgtgaagcccaatgctctgatcgactacaacaagcacatgaaaggcgtggaccgggccgaccagtacctgtcttattactctatcctgagaagaacagtgaaatggaccaagagactggccatgtacatgatcaattgcgccctgttcaacagctacgccgtgtacaagtccgtgcgacaaagaaaaatgggattcaagatgttcctgaagcagacagccatccactggctgacagacgacattcctgaggacatggacattgtgccagatctgcaacctgtgcccagcacctctggtatgagagctaagcctcccaccagcgatcctccatgtagactgagcatggacatgcggaagcacaccctgcaggccatcgtcggcagcggcaagaagaagaacatccttagacggtgcagggtgtgcagcgtgcacaagctgcggagcgagactcggtacatgtgcaagttttgcaacattcccctgcacaagggagcctgcttcgagaagtaccacaccctgaagaattactag

SEQ ID NO: 13 - protein, 572 aa, Artificial Sequence
  Synthetic Sequence
MAQHSDYSDDEFCADKLSNYSCDSDLENASTSDEDSSDDEVMVRPRTLRRRRISSSSSDSESDIEGGREEWSHVDNPPVLEDFLGHQGLNTDAVINNIEDAVKLFIGDDFFEFLVEESNRYYNQKRNNFKLSKKSLKWKDITPQEMKKFLGLIVLMGQVRKDRRDDYWTTEPWTETPYFGKTMTRDRFRQIWKAWHFNNNADIVNESDRLCKVRPVLDYFVPKFINIYKPHQQLSLDEGIVPWRGRLFFRVYNAGKIVKYGILVRLLCESDTGYICNMEIYCGEGKRLLETIQTVVSPYTDSWYHIYMDNYYNSVANCEALMKNKFRICGTIRKNRGIPKDFQTISLKKGETKFIRKNDILLQVWQSKKPVYLISSIHSAEMEESQNIDRTSKKKIVKPNALIDYNKHMKGVDRADQYLSYYSILRRTVKWTKRLAMYMINCALFNSYAVYKSVRQRKMGFKMFLKQTAIHWLTDDIPEDMDIVPDLQPVPSTSGMRAKPPTSDPPCRLSMDMRKHTLQAIVGSGKKKNILRRCRVCSVHKLRSETRYMCKFCNIPLHKGACFEKYHTLKNY

SEQ ID NO: 14 - DNA, 1719 bp, Artificial Sequence
  Synthetic Sequence
atggcccagcacagcgactaccccgacgacgagttcagagccgataagctgagtaactacagctgcgacagcgacctggaaaacgccagcacatccgacgaggacagctctgacgacgaggtgatggtgcggcccagaaccctgagacggagaagaatcagcagctctagcagcgactctgaatccgacatcgagggcggccgggaagagtggagccacgtggacaaccctcctgttctggaagattttctgggccatcagggcctgaacaccgacgccgtgatcaacaacatcgaggatgccgtgaagctgttcataggagatgatttctttgagttcctggtcgaggaatccaaccgctattacaaccagaatagaaacaacttcaagctgagcaagaaaagcctgaagtggaaggacatcacccctcaggagatgaaaaagttcctgggactgatcgttctgatgggacaggtgcggaaggacagaagggatgattactggacaaccgaaccttggaccgagaccccttactttggcaagaccatgaccagagacagattcagacagatctggaaagcctggcacttcaacaacaatgctgatatcgtgaacgagtctgatagactgtgtaaagtgcggccagtgttggattacttcgtgcctaagttcatcaacatctataagcctcaccagcagctgagcctggatgaaggcatcgtgccctggcggggcagactgttcttcagagtgtacaatgctggcaagatcgtcaaatacggcatcctggtgcgccttctgtgcgagagcgatacaggctacatctgtaatatggaaatctactgcggcgagggcaaaagactgctggaaaccatccagaccgtcgtttccccttataccgacagctggtaccacatctacatggacaactactacaattctgtggccaactgcgaggccctgatgaagaacaagtttagaatctgcggcacaatcagaaaaaacagaggcatccctaaggacttccagaccatctctctgaagaagggcgaaaccaagttcatcagaaagaacgacatcctgctccaagtgtggcagtccaagaaacccgtgtacctgatcagcagcatccatagcgccgagatggaagaaagccagaacatcgacagaacaagcaagaagaagatcgtgaagcccaatgctctgatcgactacaacaagcacatgaaaggcgtggaccgggccgaccagtacctgtcttattactctatcctgagaagaacagtgaaatggaccaagagactggccatgtacatgatcaattgcgccctgttcaacagctacgccgtgtacaagtccgtgcgacaaagaaaaatgggattcaagatgttcctgaagcagacagccatccactggctgacagacgacattcctgaggacatggacattgtgccagatctgcaacctgtgcccagcacctctggtatgagagctaagcctcccaccagcgatcctccatgtagactgagcatggacatgcggaagcacaccctgcaggccatcgtcggcagcggcaagaagaagaacatccttagacggtgcagggtgtgcagcgtgcacaagctgcggagcgagactcggtacatgtgcaagttttgcaacattcccctgcacaagggagcctgcttcgagaagtaccacaccctgaagaattactag

SEQ ID NO: 15 - protein, 572 aa, Artificial Sequence
  Synthetic Sequence
MAQHSDYPDDEFRADKLSNYSCDSDLENASTSDEDSSDDEVMVRPRTLRRRRISSSSSDSESDIEGGREEWSHVDNPPVLEDFLGHQGLNTDAVINNIEDAVKLFIGDDFFEFLVEESNRYYNQNRNNFKLSKKSLKWKDITPQEMKKFLGLIVLMGQVRKDRRDDYWTTEPWTETPYFGKTMTRDRFRQIWKAWHFNNNADIVNESDRLCKVRPVLDYFVPKFINIYKPHQQLSLDEGIVPWRGRLFFRVYNAGKIVKYGILVRLLCESDTGYICNMEIYCGEGKRLLETIQTVVSPYTDSWYHIYMDNYYNSVANCEALMKNKFRICGTIRKNRGIPKDFQTISLKKGETKFIRKNDILLQVWQSKKPVYLISSIHSAEMEESQNIDRTSKKKIVKPNALIDYNKHMKGVDRADQYLSYYSILRRTVKWTKRLAMYMINCALFNSYAVYKSVRQRKMGFKMFLKQTAIHWLTDDIPEDMDIVPDLQPVPSTSGMRAKPPTSDPPCRLSMDMRKHTLQAIVGSGKKKNILRRCRVCSVHKLRSETRYMCKFCNIPLHKGACFEKYHTLKNY

SEQ ID NO: 16 - DNA, 1732 bp, Artificial Sequence
  Synthetic Sequence
tcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattc

(19) United States
(12) Patent Application Publication
(10) Pub. No.: US 2023/0193319 A1
(43) Pub. Date: Jun. 22, 2023

(54) COMPOSITIONS AND METHODS FOR TREATMENT OF INHERITED MACULAR DEGENERATION

(71) Applicant: Saliogen Therapeutics, Inc., Cambridge, MA (US)

(72) Inventors: Joseph J. HIGGINS, Cambridge, MA (US); Scott MCMILLAN, Cambridge, MA (US); Ray TABIBIAZAR, Cambridge, MA (US)

(21) Appl. No.: 17/922,119

(22) PCT Filed: Apr. 29, 2021
(86) PCT No.: PCT/US2021/030007

§ 371 Date: Oct. 28, 2022

Related U.S. Application Data
(60) Provisional application No. 63/017,442, filed on Apr. 29, 2020.

(51) Int. Cl. C12N 15/90; A61K 48/00; A61K 9/00; A61K 9/51; A61P 27/02

(52) U.S. Cl. CPC C12N 15/90; A61K 48/0058; A61K 48/0075; A61K 9/0048; A61K 9/0019; A61K 9/5123; A61P 27/02

(57) ABSTRACT

Gene therapy compositions and methods are provided for targeting an ATP binding cassette subfamily A member 4 (ABCA4), or a functional fragment thereof, in a patient, thereby treating or mitigating Inherited Macular Degenerations including a Stargardt disease or other diseases that involve retinal degeneration.

### FIELD OF THE INVENTION

The present invention relates, in part, to methods, compositions, and products for therapy, e.g. treating and/or mitigating Inherited Macular Degeneration (IMD).

### PRIORITY

The present application claims priority to and benefit from the U.S. Provisional Patent Application No. 63/017,442 filed Apr. 29, 2020, the entirety of which is incorporated by reference herein.

### DESCRIPTION OF THE TEXT FILE SUBMITTED ELECTRONICALLY

This application contains a Sequence Listing in ASCII format submitted electronically herewith via EFS-Web. The ASCII copy, created on Apr. 28, 2021, is named SAL-002PR_Sequence_Listing_ST25.txt and is 64,498 bytes in size. The Sequence Listing is incorporated herein by reference in its entirety.

### BACKGROUND

Macular Degeneration is a condition in which cells of the macula, found in the center of the retina—the tissue at the back of the eye that senses light—become damaged, Vision loss usually occurs gradually and typically affects both eyes at different rates. Inherited Macular Degeneration (IMD), also called Macular Dystrophy (MD) refers to a group of heritable disorders that cause ophthalmoscopically visible abnormalities in the retina.

Stargardt disease (STGD), first described by the German ophthalmologist Karl Stargardt in 1909, is the most common form of IMD. It is usually is an inherited recessive disorder of the retina. Other names for the disease include Stargardt's macular dystrophy (SMD), juvenile macular degeneration, or fundus flavimaculatus. STGD typically causes vision loss during childhood or adolescence, although sometimes vision loss may not be noticed until later in adulthood. STGD causes progressive damage—or degeneration—of the macula, which is a small area in the center of the retina that is responsible for sharp, straight-ahead vision. Worldwide incidence of STGD is estimated to be 1 in 8,000-10,000 individuals.

STGD is one of several genetic disorders that cause macular degeneration, and it is characterized by a progressive worsening of vision due to the loss of light-sensing photoreceptor cells in the retina. The loss of central vision dramatically reduces one's ability to read, write, and navigate the surrounding environment, significantly reducing the person's quality of life. Recessive Stargardt disease (STGD1) is by far the most common form of Stargardt disease, which is caused by mutations in the ATP binding cassette subfamily A member 4 (ABCA4). The ABCA4 gene/protein is expressed in photoreceptor (PR) cells. STGD1 is manifested by deposition of lipofuscin, a fluorescent mixture of partially digested proteins and lipids, in the lysosomal compartment of the retinal pigment epithelium (RPE), which precedes photoreceptor degeneration. RPE plays a role in controlling the immune response through expression of mRNAs and proteins associated with the complement portion of the immune system, which is a key component of innate immunity. Age-related macular degeneration (AMD) is a disease with significant similarities to STGD1, and it is also associated with RPE lipofuscin accumulation and complement dysregulation. Lenis et al., Proc Natl Acad Sci USA. 2017 Apr. 11; 114(15):3987-3992.

Another form of STGD is STGD4, a rare dominant defect in the PROM1 gene. Kniazeva et al. Am J Hum Genet. 1999; 64:1394-1399. STGD3, also known as Stargardt-like dystrophy, is another rare dominant form of STGD, caused by mutations in the Elongation of Very Long-Chain Fatty Acids-Like 4 Gene (ELOVL4). Agbaga et al. Invest Ophthalmol Vis Sci. 2014; 55: 3669-3680.

Existing therapies for IMD include deuterated vitamin A, microcurrent stimulation (MCS), RPE transplantation, nutritional supplements, stem cell therapy, and modulation of the complement system. Despite these efforts, however, currently there is no effective therapy for treatment of IMD in general arid STGD in particular. Gene therapy development for IMD diseases has been challenging because commonly used adeno-associated viruses (AAVs) do not have the capacity for a gene with a coding sequence larger than 5 kb, which includes ABCA4 (6.8 kb), the gene responsible for STGD, among other retinal disorders.

Accordingly, compositions and methods for efficiently preventing and treating IMD such as Stargardt disease, as well as other macular dystrophies, are needed.

### SUMMARY

In various aspects, the present invention provides compositions and methods for treating and/or mitigating Inherited Macular Degeneration (IMD) disorders, which are a major cause of blindness worldwide. IMD includes Stargardt disease and other Macular dystrophies (MDs), including Best disease, X-linked retinoschisis, pattern dystrophy, Sorsby fundus dystrophy and autosomal dominant drusen. The compositions and methods of the present invention make use of gene transfer constructs comprising transposon expression vectors that use sequence- or locus-specific transposition (SLST) to correct gene defects associated with these diseases. The described compositions and methods employ a non-viral mode of gene transfer. Thus, shortcomings associated with use of viral vectors are overcome.

In some aspects, a composition comprising a gene transfer construct is provided that comprises (a) a nucleic acid encoding an ATP binding cassette. subfamily A member 4 (ABCA4) protein, or a functional fragment thereof; (b) retina-specific promoter, and (c) a non-viral vector comprising one or more transposase recognition sites and one or more inverted terminal repeats (ITRs) or end sequences.

The gene therapy in accordance with the present disclosure can be performed using transposon-based vector systems, with the assistance by transposases, which are provided on the same vector as the gene to be transferred (cis) or on a different vector (trans) or as RNA. The transposon-based vector systems can operate under the control of a retina-specific promoter.

In embodiments, the transposase, e.g. one derived from Bombyx mori, Xenopus tropicalis, Trichoplusia ni, Rhinolophus ferrumequinum, Rousettus aegyptiacus, Phyllostomus discolor, Myotis myotis, Myotis lucifugus, Pteropus vampyrus, Pipistrellus kuhlii, Pan troglodytes, Molossus molossus, or Homo sapiens, and/or is an engineered version thereof, is used to insert the ABCA4 gene, or a functional fragment thereof, into a patient's genome.

In embodiments, a transposase is a Myotis lucifugus transposase (MLT, or MLT transposase), which comprises an amino acid sequence of SEQ ID NO: 10, or a variant having at least about 90%, or at least about 93%, or at least about 95%, or at least about 97%, or at least about 98%, or at least about 99% identity thereto, and one or more mutations selected from 1573X, E574X, and S2X, wherein X is any amino acid or no amino acid, optionally X is A, G, or a deletion. In embodiments, the mutations are L573del, E574del, and S2A.

In embodiments, the MLT transposase comprises an amino acid sequence with mutations L573del, E574del, and S2A (SEQ ID NO: 10), and additionally with one or more mutations that confer hyperactivity (or hyperactive mutations). In embodiments, the hyperactive mutations are one or more of S8X, C13X, and N125X mutations, wherein X is optionally any amino acid or no amino acid, optionally X is P, R, or K. In embodiments, the mutations are S8P, C13R, and N125K. In some embodiments, the MLT transposase has S8P and C13R mutations, In some embodiments, the MLT transposase has N125K mutation, in some embodiments, the MLT transposase has all three S8P, C13R, and N125K mutations.

The described compositions can be delivered to a host cell using lipid nanoparticles (LNPs). In some embodiments, the LNP comprises one or more molecules selected from a neutral or structural lipid (e.g. DSPC), cationic lipid (e.g. MC3), cholesterol, PEG-conjugated lipid (CDM-PEG), and a targeting ligand (e.g. N-Acetylgalactosamine (GalNAc)), In some embodiments, the LNP comprises GalNAc or another ligand for Asialoglycoprotein Receptor (ASGPR)-mediated uptake into cells with mutated ABCA4 or other genes (e.g., ELOVL4, PROM1, BEST1, or PRPH2).

In some aspects, a method for preventing or decreasing the rate of photoreceptor loss in a patient is provided, which can be an in vivo or ex vivo method. Accordingly, in some embodiments, a method is provided that comprises administering to a patient in need thereof a composition in accordance with embodiments of the present disclosure. In some embodiments, an ex vivo method for preventing or decreasing the rate of photoreceptor loss in a patient is provided that comprises (a) contacting a cell obtained from a patient (autologous) or other individual (allogeneic) with the described composition, and (b) administering the cell to a patient in need thereof.

In some embodiments, a method for treating and/or mitigating a class of IMDs (also referred to as Macular dystrophies (MDs)) is provided, including STGD, Best disease, X-linked retinoschisis, pattern dystrophy, Sorsby fundus dystrophy and autosomal dominant drusen.

In some embodiments, a method for treating and/or mitigating an IMD is provided, which can also be performed in vivo or ex vivo. In some embodiments, the method comprises administering to a patient in need thereof composition in accordance with embodiments of the present disclosure, in some embodiments, the method for treating and/or mitigating an IMD comprises (a) contacting a cell obtained from a patient or another individual with a composition of the present disclosure, and (b) administering the cell to a patient in need thereof.

The IMD can be a STGD, and, in some embodiments, the STGD can be STGD Type 1 (STGD1). In some embodiments, the STGD can be STGD Type 3 (STGD3) or STGD Type 4 (STGD4) disease. The IMD can be characterized by one or more mutations in one or more of ABCA4, ELOVL4, PROM1, BEST1, and PRPH2. The ABCA4 mutations can be autosomal recessive or dominant mutations. The methods in accordance with the present disclosure allow reducing, decreasing, or alleviating symptoms of IMD such as, e.g. Stargardt disease, including improved distance visual acuity and/or decreased the rate of photoreceptor loss as compared to a lack of treatment. In some embodiments, the method results in improvement of best corrected visual acuity (BCVA) to greater than about 20/200.

The compositions and methods in accordance with embodiments of the present disclosure are substantially non-immunogenic, do not cause any unmanageable side effects, and, in some cases, can be effectively delivered via a single administration. The prevention or decreasing of the rate of photoreceptor loss can be robust and durable. The described compositions and methods lower or prevent lipofuscin accumulation in the retina (e.g., in the RPE and/or Bruch's membrane), reduce or prevent formation of retinal pigment epithelium (RPE) debris, improve distance visual acuity of the patient.

In some aspects of the present disclosure, an isolated cell is provided that comprises the composition in accordance with embodiments of the present disclosure.

In some embodiments, the method provides improved distance visual acuity and/or decreased the rate of photoreceptor loss as compared to a lack of treatment. The method can also result in improvement of best corrected visual acuity (BCVA) to greater than about 20/200. In some embodiments, the method results in improvement of retinal or foveal morphology, as measured by fundus autofluorescence (FAF) or Spectral Domain-Optical Coherence Tomography (SD-OCT). Other imaging technologies can be used as well.

The described method improve patients vision. In some embodiments, the methods result in reduction or prevention of one or more of wavy vision, blind spots, blurriness, loss of depth perception, sensitivity to glare, impaired color vision, and difficulty adapting to dim lighting (delayed dark adaptation) in the patient.

In some embodiments, the methods in accordance with the present disclosure obviate the need for steroid treatment. Additionally or alternatively, the methods can obviate the need for Soraprazan, Isotretinoin, Dobesilate, 4-methylpyrazole, ALK-001 9 (C20 deuterated vitamin A), Fenretinide (a synthetic form of vitamin A), LBS-500, A1120, Emixustat, Fenofibrate, Avacincaptad pegol, and other therapeutic agents. In some embodiments, however, the present compositions and methods involve the use of one or more additional therapeutic agents selected from Soraprazan, Isotretinoin, Dobesilate, 4-methylpyrazole, ALK-001 9 (020 deuterated vitamin A), Fenretinide (a synthetic form of vitamin A), LBS-500, A1120, Emixustat, Fenofibrate, Avacincaptad pegol, and other therapeutic agents. Other aspects and certain embodiments of the invention will be apparent from the following detailed description.

### BRIEF DESCRIPTION OF THE FIGURES

FIGS. 1A, 1B, 1C, 1D, 1E, 1F, 1J, 1H, and 1I are schematic representations of the vectors that can be used in the transfection, transposition efficacy, and expression studies in retinal cell lines.

FIG. 2 illustrates a lipid nanoparticle structure used in some embodiments of the present disclosure.

FIG. 3 shows GFP expression of 661W mouse photoreceptor cells 24 hours post transfection with varying lipofection reagents as well as either MLT transposase 1 (MLT with the N125K mutation) or MLT transposase 2 (MLT with the S8P/C13R mutations) of the present disclosure, compared to un-transfected cells. The top row shows un-transfected 661W mouse photoreceptor cells, cells transfected with a transposon with L3 (Lipofectamine 3000) and MLT 1, and cells transfected with a transposon with L3 and MLT 2; the middle row shows un-transfected 661W mouse photoreceptor cells, cells transfected with a transposon with LTX (Lipofectamine LTX & PLUS) and MLT 1, and cells transfected with a transposon with LTX and MLT 2; and the bottom row shows un-transfected 661W mouse photoreceptor cells, cells transfected with a transposon with MAX (Lipofectamine Messenger MAX) and MLT 1, and cells transfected with a transposon with MAX and MLT 2.

FIG. 4 shows stable integration of donor DNA (GFP) by transposition in mouse photoreceptor cell line 661W after 4 rounds of splitting over 15 days. The rows show results for days 3, 6, 9, 12, and 15; the columns show results for untransfected cells, cells transfected with a donor DNA only; cells transfected with a donor DNA and MLT 1, and cells transfected with a donor DNA and MLT 2.

FIG. 5 is a bar chart illustrating results of FACS analysis of stable integration of a donor DNA (GFP) by transposition in mouse photoreceptor cell line 661W on day 15. The percent (%) of OFF expression is shown for untransfected cells, cells transfected with the donor DNA only (“+GFP only”); cells transfected with the donor DNA and MLT 1 (“MLT 1+GFP”); and cells transfected with the donor DNA and MLT 2 (“MLT 2+GFP”).

FIG. 6 shows expression of GFP in ARPE-19 cells at 24 hours post transfection. The top row shows un-transfected ARPE-19 cells, cells transfected with a transposon with L3 only, cells transfected with a transposon with L3 and MLT 1, and cells transfected with a transposon with L3 and MLT 2; the middle row shows un-transfected ARPE-19 cells, cells transfected with a transposon with LTX only, cells transfected with a transposon with LTX and MLT 1, and cells transfected with a transposon with LTX and MLT 2; and the bottom row shows un-transfected ARPE-19 cells, cells transfected with a transposon with MAX only, cells transfected with a transposon with MAX and MLT 1, and cells transfected with a transposon with MAX and MLT 2.

FIG. 7 shows higher resolution images of MLT transposase 1 and MLT transposase 2, visible GFP expression at 24 hours post transfection.

FIG. 8 shows stable integration of donor DNA (GFP) in photoreceptor cell line ARPE19 with MLT transposase 2 (MLT 2). The rows show results for days 4, 8, 12, and 15; the columns show results for cells transfected with a donor DNA only, and cells transfected with the donor DNA and MLT 2.

FIG. 9 is a bar chart illustrating results of FACS analysis of stable integration of a donor DNA (GFP) by transposition in ARPE19 cell lines after 4 generations of cell divisions. The percent (%) of GFP expression is shown for untransfected cells, cells transfected with the donor DNA only (“+GFP only”); cells transfected with the donor DNA and MLT 1 (“MLT 1+GFP”); and cells transfected with the donor DNA and MLT 2 (“MLT 2+GFP”).

FIGS. 10A and 10B depict images of mouse 1-1L left (FIG. 10A) and 1-1L right (FIG. 10B) eyes injected with PBS.

FIGS. 11A, 11B, 11C, and 11D depict images of mice 3-1L and 3-1R right eyes injected with only DNA (FIG. 11A and FIG. 11C) and mice 3-1L and 3-1R left eyes injected with a donor DNA and MLT 2 (FIG. 11B and FIG. 11D).

FIGS. 12A and 12B depict images of mouse 4-1R's right eye injected with a donor DNA (FIG. 12A) and MLT 2 (FIG. 12B).

FIGS. 13A and 13B depict images of mouse 4-NP right eye (FIG. 13A) injected with only a donor DNA, and left eye (FIG. 13B) injected with both the donor DNA and MLT 2.

FIGS. 14A and 14B depict images of mouse 4-1L right eye (FIG. 14A) injected with only a donor DNA, and left eye (FIG. 14B) injected with both the donor DNA and MLT 2.

FIGS. 15A and 15B depict images of mouse 5-BP right eye (FIG. 15A) injected with only a donor DNA, and left eye (FIG. 15B) injected with both the donor DNA and MLT 2.

FIG. 16 illustrates a design of experiments that assess effectiveness of transposition of 661W mouse photoreceptor cells and retinal epithelium (ARPE19) cells using a DNA donor and an RNA helper in accordance with some embodiments of the present disclosure.

FIG. 17 depicts images of mouse left and right eyes (top and bottom rows, respectively), taken on day 21 day post sub-retinal injection, with (“+MLT”) or without (“−MLT”) the MLT transposase used in the transfection.

### DETAILED DESCRIPTION

The present invention is based, in part, on the discovery that non-viral, capsid free gene therapy methods and compositions can be used for preventing or decreasing the rate of photoreceptor loss in a patient. The non-viral gene therapy methods in accordance with the present disclosure find use in retina-directed gene therapy for Inherited Macular Degenerations (IMDs). In some embodiments, the present methods and compositions find use in retina-directed gene therapy for Stargardt disease (STGD) caused by mutations in an ATP binding cassette subfamily A member 4 (ABCA4). The described methods and compositions employ transposition of ABCA4 or another gene or a functional fragment thereof, from a gene transfer construct to a host genome. The described methods and compositions lower or prevent lipofuscin accumulation in the retina (e.g., in the RPE and/or Bruch's membrane, and photoreceptors), and improve distance visual acuity of the patient.

STGD is characterized by macular atrophy and peripheral flecks in the retinal pigment epithelium (RPE). The ABCA4 gene encodes a protein (ABCA4 protein) found in rod and cone photoreceptors, which is a transmembrane protein involved in the transport of vitamin A intermediates, such as specifically N-retinylidine-phosphatidylethanol-amine (N-RPE), to the RPE. ABCA4 is responsible for the clearance of all-trans-retinal (reactive vitamin A aldehyde) from photoreceptor cells, and loss of ABCA4 function leads to the accumulation of bis-retinoids (such as N-RPE) in the outer segment membranes of the photoreceptor cells, which in turn causes the formation of lipofuscin. This ultimately leads to accumulation of high levels of lipofuscin in the RPE (and thus increased retinal autofluorescence) and progressive RPE and photoreceptor cell loss.

Mutations of ABCA4 are associated with a wide spectrum of phenotypes, including cone-rod dystrophy (cones and rods die away in STGD disease) and retinitis pigmentosa (a breakdown and loss of cells in the retina). See, e.g., Song et al., JAMA Ophthalmol. 2015; 133(10):1198-1203. Similarly, mutations in other genes responsible for MDs similarly exhibit various phenotypes that differ among patients.

As mentioned above, the use of the adeno-associated virus (AAV) vector for gene therapy involving ABCA4 is prevented by the size of ABCA4 (6.8 kb) that exceeds the 4.5 kb to 5.0 kb capacity of the AAV. Thus, equine infectious anemia lentivirus (EIAV) has been used for gene transfer, by subretinal injection. Kong et al., Gene Ther 2003; 15(10):1311-1320. Another approach that addressed the relatively large size of ABCA4 was to split the gene across two AAV vectors such that the two transgene fragments combine inside the host cell. Dyka et al., Hum Gene Ther 2019; November; 30(11):1361-1370.

The compositions and methods of the present disclosure provide a non-viral delivery of transgenes that replace mutated copies of ABCA4 or other targeted gene(s). Accordingly, the compositions and methods of the present disclosure provide gene transfer constructs that target ABCA4, or a functional fragment thereof, to correct pathogenic variants in the patient's genome and to thus prevent or decrease the rate of photoreceptor loss in a patient. Accordingly, in some aspects of the present disclosure, a composition comprising a gene transfer construct is provided, comprising (a) a nucleic acid encoding ABCA4 protein, or a functional fragment thereof, (b) a retina-specific promoter, and (c) a non-viral vector comprising one or more transposase recognition sites and one or more inverted terminal repeats (ITRs) or end sequences.

In some embodiments, the ABCA4 protein is human ABCA4 protein, or a functional fragment thereof. In embodiments, a gene encoding the human ABCA4 is human ABCA4 (GenBank Ace. No. NM_000350). The nucleic acid encoding the human ABCA4 may comprise a nucleotide sequence encoding a protein having an amino acid sequence of SEQ ID NO: 1, or a variant having at least about 90%, or at least about 93%, or at least about 95%, or at least about 97%, or at least about 98% identity thereto. In some embodiments, the nucleic acid encoding the human ABCA4 comprises a nucleotide sequence of SEQ ID NO: 2, or a variant having at least about 90%, or at least about 93%, or at least about 95%, or at least about 97%, or at least about 98% identity thereto.

In some embodiments, the nucleic acid encoding the human ABCA4 comprises a nucleotide sequence encoding a protein having an amino add sequence of SEQ ID NO: 1, or a variant having at least about 90%, or at least about 93%, or at least about 95%, or at least about 97%, or at least about 98% identity thereto. In some embodiments, the nucleic acid encoding the human ABCA4 comprises a nucleotide sequence of SEQ ID NO: 2, or a variant haying at least about 90%, or at least about 93%, or at least about 95%, or at least about 97%, or at least about 98% identity thereto. SEQ ID NO: 1 is

```
(SEQ ID NO: 1)
      1 MGFVRQIQLL LWKNWTLRKR QKIRFVVELV WPLSLFLVLI WLRNANPLYS HHECHFPNKA
    61 MPSAGMLPWL QGIFCNVNNP CFQSPTPGES PGIVSNYNNS ILARVYRDFQ ELLMNAPESQ
  121 HLGRIWTELH ILSQFMDTLR THPERIAGRG IRIRDILKDE ETLTLFLIKN IGLSDSVVYL
  181 LINSQVRPEQ FAHGVPDLAL KDIACSEALL ERFIIFSQRR GAKTVRYALC SLSQGTLQWI
  241 EDTLYANVDF FKLFRVLPTL LDSRSQGINL RSWGGILSDM SPRIQEFIHR PSMQDLLWVT
  301 RPLMQNGGPE TFTKLMGILS DLLCGYPEGG GSRVLSFNWY EDNNYKAFLG IDSTRKDPIY
  361 SYDRRTTSFC NALIQSLESN PLTKIAWRAA KPLLMGKILY TPDSPAARRI LKNANSTFEE
  421 LEHVRKLVKA WEEVGPQIWY FFDNSTQMNM IRDTLGNPTV KDFLNRQLGE EGITAEAILN
  481 FLYKGPRESQ ADDMANFDWR DIFNITDRTL RLVNQYLECL VLDKFESYND ETQLTQRALS
  541 LLEENMFWAG VVFPDMYPWT SSLPPHVKYK IRMDIDVVEK TNKIKDRYWD SGPRADPVED
  601 FRYIWGGFAY LQDMVEQGIT RSQVQAEAPV GIYLOOMPYP CFVDDSFMII LNRCFPIFMV
  661 LAWIYSVSMT VKSIVLEKEL RLKETLKNQG VSNAVIWCTW FLDSFSIMSM SIFLLTIFIM
  721 HGRILHYSDP FTLFLFLLAF STATIMLCFL LSTFFSKASL AAACSGVIYE TLYLPHILCE
  781 AWQDRMTAEL KKAVSLLSPV AFGFGTEYLV RFEEQGLGLQ WSNIGNSPTE GDEFSFLLSM
  841 QMMLLDAAVY GLLAWYLDQV FPGDYGTPLP WYFLLQESYW LGGEGCSTRE ERALEKTEPL
  901 TEETEDPEHP EGIHDSFFER EHPGWVPGVC VKNLVKIFEP CGRPAVDRLN ITFYENQITA
  961 FLGHNGAGKT TTLSILTGLL PPTSGTVLVG GRDIETSLDA VROSLGMCPQ HNILFHHLTV
1021 AEHMLFYAQL KGKSQEEAQL EMEAMLEDTG LHHKRNEEAQ DLSGGMQRKL SVAIAFVGDA
1081 KVVILDEPTS GVDPYSRRSI WDLLLKYRSG RTIIMSTHHM DEADLLGDRI AIIAQGRLYC
1141 SGTPLFLKNC FGTGLYLTLV RKMKNIQSQR KGSEGTCSCS SKGFSTTCPA HVDDLTPEQV
1201 LDGDVNELMD VVLHHVPEAK LVECIGQELI FLLPNKNFKH RAYASLFREL EETLADLGLS
1261 SFGISDTPLE EIFLKVTEDS DSGPLFAGGA QQKRENVNPR HPCLGPREKA GQTPQDSNVC
1321 SPGAPAAHPE GQPPPEPECP GPQLNTGTQL VLQHVQALLV KRFQHTIRSH KDFLAQIVLP
1381 ATFVFLALML SIVIPPFGEY PALTLHPWIY GQQYTFFSMD EPGSEQFTVL ADVLLNKPGE
1441 GNRCLKEGWL PEYPCGNSTP WKTPSVSPNI TQLFQKQKWT QVNPSPSCRC STREKLTMLP
1501 ECPEGAGGLP PPQRTQRSTE ILQDLTDRNI SDFLVKTYPA LIRSSLKSKE WVNEQRYGGI
1561 SIGGKLPVVP ITGEALVGFL SDLGRIMNVS GGPITREASK EIPDFLKHLE TEDNIKVWFN
1621 NKGWHALVSF LNVAHNAILR ASLPKDRSPE EYGITVISQP LNLTKEQLSE ITVLTTSVDA
1681 VVAICVIFSM SFVPASFVLY LIQERVNKSK HLQFISGVSP TTYWVTNFLW DIMNYSVSAG
1741 LVVGIFIGFQ KKAYTSPENL PALVALLLLY GWAVIPMMYP ASFLFDVPST AYVALSCANT
1801 FIGINSSAIT FILELFENNR TLLRFNAVLR KLLIVFPHFC LGRGLIDLAL SQAVTDVYAR
1861 FGEEHSANPF HWDLIGKNLF AMVVEGVVYE LLTLLVQRHF FLSQWIAEPT KEPIVDEDDD
1921 VAEERQRIIT GGNKTDILRL HELTKIYPGT SSPAVDRLCV GVRPGECFGL LGVNGAGKTT
1981 TFKMLTGDTT VTSGDATVAG KSILTNISEV HQNMGYCPQF DAIDELLTGR EHLYLYARLR
2041 GVPAEEIEKV ANWSIKSLGL TVYADCLAGT YSGGNKRKLS TAIALIGCPP LVLLDEPTTG
2101 MDPQARRMLW NVIVSIIREG RAVVLTSHSM EECEALCTRL AIMVKGAFRC MGTIQHLKSK
2161 FGDGYIVTMK IKSPKDDLLP DLNPVEQFFQ GNFPGSVQRE RHYNMLQFQV SSSSLARIFQ
2221 LLLSHKDSLL IEEYSVTQTT LDQVFVNFAK QQTESHDLPL HPRAAGASRQ AQD
```

In embodiments, the human ABCA4 is encoded by a nucleotide sequence of SEQ ID NO: 2, or a variant having at least about 90%, or at least about 93%, or at least about 95%, or at least about 97%, or at least about 98% identity thereto, including a codon-optimized version. SEQ ID NO: 2 is:

| (SEQ ID NO: 2) | |
|---|---|
| 1 | atgggcttcg tgagacagat acagcttttg ctccggaaga actggaccct gaggaaaagg |
| 61 | caaaagattc gctttgtggt ggaactcgtg tggcctttat ctttatttct ggtcttgatc |
| 121 | tggttaagga atgccaaccc actctacagc catcatgaat gccatttccc caacaaggcg |
| 181 | atgccctcag cagcaatgct gccgtggctc caggggatct tctgcaatgt gaacaatccc |
| 241 | tgttttcaaa gccccacccc aggagaatct cctggaattg tgtcaaacta taacaactcc |
| 301 | atcttggcaa gggtatatcg agattttcaa gaactcctca tgaasccacc agagagccag |
| 361 | caccttggcc gtatttggac agagctacac atcttgtccc aattcatgga caccctccgg |
| 421 | actcacccgg agagaattgc aggaagagga atacgaataa gggatatctt gaaagatgaa |
| 481 | gaaacactga cactatttct cattaaaaac atcggcctgt ctgactcagt ggtctacctt |
| 541 | ctgatcaact ctcaagtccg tccagagcag ttcgctcatg gagtcccgga cctggcgctg |
| 601 | aaggacatcg cctgcagcga ggccctcctg gagcgcttca ccatcttcag ccagagacgc |
| 661 | ggggcaaaga cggtgcgcta tgccctgtgc tccctctccc agggcaccct acagtggata |
| 721 | gaagacactc tgtatgccaa cgtggacttc ttcaagctct tccgtgtgct tcccacactc |
| 781 | ctagacagcc gttctcaagg tatcaatctg agatcttggg gaggaatatt atctgatatg |
| 841 | tcaccaagaa ttcaagagtt tatccatcgg ccgagtatgc aggacttgct gtgggtgacc |
| 901 | aggcccctca tgcagaatgg tggtccagag acctttacaa agctgatggg catcctgtct |
| 961 | gacctcctgt gtggctaccc cgagggaggt ggctctcggg tgctctcctt caactggtat |
| 1021 | gaagacaata actataaggc ctttctgggg attgactcca caaggaagga tcctatctat |
| 1081 | tcttatgaca gaagaacaac atccttttgt aatgcattga tccagagcct ggagtcaaat |
| 1141 | cctttaacca aaatcgcttg gagggcggca aagcctttgc tgatgggaaa aatcctgtac |
| 1201 | actcctgatt cacctgcagc acgaaggata ctgaagaatg ccaactcaac ttttgaagaa |
| 1261 | ctggaacacg ttaggaagtt ggtcaaagcc tgggaagaag tagggcccca gatctggtac |
| 1321 | ttctttgaca acagcacaca gatgaacatg atcagagata ccctggggaa cccaacagta |
| 1381 | aaagactttt tgaataggca gcttggtgaa gaaggtatta ctgctgaagc catcctaaac |
| 1441 | ttcctctaca agggccctcg ggaaagccag gctgacgaca tggccaactt cgactggagg |
| 1501 | gacatattta acatcactga tcgcaccctc cacctagtca atcaatacct ggagtgcttg |
| 1561 | gtcctggata agtttgaaag ctacaatgat gaaactcagc tcacccaacg tgccctctct |
| 1621 | ctactggagg aaaacatgtt ctgggccgga gtggtattcc ctgacatgta tccctggacc |
| 1681 | agctctctac caccccacgt gaagtataag atccgaatgg acatagacgt ggtggagaaa |
| 1741 | accaataaga ttaaagacag gtattgggat tctggtccca gagctgatcc cgtggaagat |
| 1801 | ttccggtaca tctggggcgg gtttgcctat ctgcaggaca tggttgaaca ggggatcaca |
| 1861 | aggagccagg tgcaggcgga ggctccagtt ggaatctacc tccagcagat gccctacccc |
| 1921 | tgcttcgtgg acgattcttt catgatcatc ctgaaccgct gtttccctat cttcatggtg |
| 1981 | ctggcatgga tctactctgt ctccatgact gtgaagagca tcgtcttgga gaaggagttg |
| 2041 | cgactgaagg agaccttgaa aaatcagggt gtctccaatg cagtgatttg gtgtacctgg |
| 2101 | ttcctggaca gcttctccat catgtcgatg agcatcttcc tcctgacgat attcatcatg |
| 2161 | catggaagaa tcctacatta cagcgaccca ttcatcctct tcctgttctt gttggctttc |
| 2221 | tccactgcca ccatcatgct gtgctttctg ctcagcacct tcttctccaa ggccagtctg |
| 2281 | gcagcagcct gtagtggtgt catctatttc accctctacc tgacacacat cctgtgcttc |
| 2341 | gcctggcagg accgcatgac cgctgagctg aagaaggctg tgagcttact gtctccggtg |
| 2401 | gcatttggat ttggcactga gtacctggtt cgctttgaag accaaggccc ggggctgcag |
| 2461 | tggagcaaca tcgggaacag tcccacggaa ggggacgaat tcagcttcct gctgtccatg |
| 2521 | cagatgatgc tccttgatgc tgctgtctat ggcttactcg cttggtacct tgatcaggtg |
| 2581 | tttccaggag actatggaac cccacttcct tggtactttc ttctacaaga gtcgtattgg |
| 2641 | cttggcggtg aagggtgttc aaccagagaa gaaagagccc tggaaaagac cgagccccta |
| 2701 | acagaggaaa cggaggatcc agagcaccca gaaggaatac acgactcctt atttgaacgt |
| 2761 | gagcatccag ggtgggttcc tggggtatgc gtgaagaatc tggtaaagat ttttgagccc |
| 2821 | tgtggccggc cagctgtgga ccgtctgaac atcaccttct acgagaacca gatcaccgca |
| 2881 | ttcctgggcc acaatggagc tgggaaaacc accaccttgt ccatcctgac gggtctgttg |
| 2941 | ccaccaacct ctgggactgt gctcgttggg ggaagggaca ttgaaaccag cctggatgca |
| 3001 | gtccggcaga gccttggcat gtgtccacag cacaacatcc tgttccacca cctcacggtg |
| 3061 | gctgagcaca tgctgttcta tgcccagctg aaaggaaagt cccaggagga ggcccagctg |
| 3121 | gagatggaag ccatgttgga ggacacaggc ctccaccaca agcggaatga agaggctcag |
| 3181 | gacctatcag gtggcatgca gagaaagctg tcggttgcca ttgcctttgt gggagatgcc |
| 3241 | aaggtggtga ttctggacga acccacctct ggggtggacc cttactcgag acgctcaatc |
| 3301 | tgggatctgc tcctgaagta tcgctcaggc agaaccatca tcatgtccac tcaccacatg |
| 3361 | gacgaggccg acctccttgg ggaccgcatt gccatcattg cccagggaag gctctactgc |
| 3421 | tcaggcaccc cactcttcct gaagaactgc tttggcacag gcttgtactt aaccttggtg |
| 3481 | cgcaagatga aaaacatcca gagccaaagg aaaggcagtg aggggacctg cagctgctcg |
| 3541 | tctaagggtt tctccaccac gtgtccagcc cacgtcgatg acctaactcc agaacaagtc |
| 3601 | ctggatgggg atgtaaatga gctgatggat gtagttctcc accatgttcc agaggcaaag |
| 3661 | ctggtggagt gcattggtca agaacttatc ttccttcttc caaataagaa cttcaagcac |
| 3721 | agagcatatg ccagcctttt cagagagctg gaggagacgc tggctgacct tggtctcagc |
| 3781 | agttttggaa tttctgacac tcccctggaa gagatttttc tgaaggtcac ggaggattct |
| 3841 | gattcaggac ctctgtttgc gggtggcgct cagcagaaaa gagaaaacgt caacccccga |
| 3901 | cacccctgct tgggtcccag agagaaggct ggacagacac cccaggactc aaatgtctgc |
| 3961 | tccccagggg cgccggctgc tcacccagag ggccagcctc ccccagagcc agagtgccca |
| 4021 | ggcccgcagc tcaacacggg gacacagctg gtcctccagc atgtgcaggc gctgctggtc |
| 4081 | aagagattcc aacacaccat ccgcagccac aaggacttcc tggcgcagat agtgctcccg |
| 4141 | gctacctttg tgtttttggc tctgatgctt tctattgtta tccctccttt tggcgaatac |
| 4201 | cccgctttga cccttcaccc ctggatatat gggcagcagt acaccttctt cagcatggat |
| 4261 | gaaccaggca gtgagcagtt cacggtactt gcagacgtcc tcctgaataa gccaggcttt |
| 4321 | ggcaaccgct gcctgaagga agggtggctt ccggagtacc cctgtggcaa ctcaacaccc |
| 4381 | tggaagactc cttctgtgtc cccaaacatc acccagctgt tccagaagca gaaatggaca |
| 4441 | caggtcaacc cttcaccatc ctgcaggtgc agcaccaggg agaagctcac catgctgcca |
| 4501 | gagtgccccg agggtgccgg gggcctcccg cccccccaga gaacacagcg cagcacggaa |
| 4561 | attctacaag acctgacgga caggaacatc tccgacttct tggtaaaaac gtatcctgct |
| 4621 | cttataagaa gcagcttaaa gagcaaattc tgggtcaatg aacagaggta tggaggaatt |
| 4681 | tccattggag gaaagctccc agtcgtcccc atcacggggg aagcacttgt tgggttttta |
| 4741 | agcgaccttg gccggatcat gaatgtgagc gggggcccta tcactagaga ggcctctaaa |
| 4801 | gaaatacctg atttccttaa acatctagaa actgaagaca acattaaggt gtggtttaat |
| 4861 | aacaaaggct ggcatgccct ggtcagcttt ctcaatgtgg cccacaacgc catcttacgg |
| 4921 | gccagcctgc ctaaggacag gagccccgag gagtatggaa tcaccgtcat tagccaaccc |
| 4981 | ctgaacctga ccaaggagca gctctcagag attacagtgc tgaccacttc agtggatgct |
| 5041 | gtggttgcca tctgcgtgat tttctccatg tccttcgtcc cagccagctt tgtcctttat |
| 5101 | ttgatccagg agcgggtgaa caaatccaag cacctccagt ttatcagtgg agtgagcccc |
| 5161 | accacctact gggtgaccaa cttcctctgg gacatcatga attattccgt gagtgctggg |
| 5221 | ctggtggtgg gcatcttcat cgggtttcag aagaaagcct acacttctcc agaaaacctt |
| 5281 | cctgcccttg tggcactgct cctgctgtat ggatgggcgg tcattcccat gatgtaccca |
| 5341 | gcatccttcc tgtttgatgt ccccagcaca gcctatgtgg ctttatcttg tgctaatctg |
| 5401 | ttcatcggca tcaacagcag tgctattacc ttcatcttgg aattatttga gaataaccgg |
| 5461 | acgctgctca ggttcaacgc cgtgctgagg aagctgctca ttgtcttccc ccacttctgc |
| 5521 | ctgggccggg gcctcattga ccttgcactg agccaggctg tgacagatgt statgcccgg |
| 5581 | tttggtgagg agcactctgc aaatccgttc cactgggacc tgattgggaa gaacctgttt |
| 5641 | gccatggtgg tggaaggggt ggtgtacttc ctcctgaccc tgctggtcca gcgccacttc |
| 5701 | ttcctctccc aatggattgc cgagcccact aaggagccca ttgttgatga agatgatgat |
| 5761 | gtggctgaag aaagacaaag aattattact ggtggaaata aaactgacat cttaaggcta |
| 5821 | catgaactaa ccaagattta tccaggcacc tccagcccag cagtggacag gctgtgtgtc |
| 5881 | ggagttcgcc ctggagagtg ctttggcctc ctgggagtga atggtgccgg caaaacaacc |
| 5941 | acattcaaga tgctcactgg ggacaccaca gtgacctcag gggatgccac cgtagcaggc |
| 6001 | aagagtattt taaccaatat ttctgaagtc catcaaaata tgggctactg tcctcagttt |
| 6061 | gatgcaattg atgagctgct cacaggacga gaacatcttt acctttatgc ccggcttcga |
| 6121 | ggtgtaccag cagaagaaat cgaaaaggtt gcaaactgga gtattaagag cctgggcctg |
| 6181 | actgtctacg ccgactgcct ggctggcacg tacagtgggg gcaacaagcg gaaactctcc |
| 6241 | acagccatcg cactcattgg ctgcccaccg ctggtgctgc tggatgagcc caccacaggg |
| 6301 | atggaccccc aggcacgccg catgctgtgg aacgtcatcg tgagcatcat cagagaaggg |
| 6361 | agggctgtgg tcctcacatc ccacagcatg gaagaatgtg aggcactgtg tacccggctg |
| 6421 | gccatcatgg taaagggcgc ctttcgatgt atgggcacca ttcagcatct caagtccaaa |
| 6481 | tttggagatg gctatatcgt cacaatgaag atcaaatccc cgaaggacga cctgcttcct |
| 6541 | gacctgaacc ctgtggagca gttcttccag gggaacttcc caggcagtgt gcagagggag |
| 6601 | aggcactaca acatgctcca gttccaggtc tcctcctcct ccctggcgag gatcttccag |
| 6661 | ctcctcctct cccacaagga cagcctgctc atcgaggagt actcagtcac acagaccaca |
| 6721 | ctggaccagg tgtttgtaaa ttttgctaaa cagcagactg aaagtcatga cctccctctg |
| 6781 | caccctcgag ctgctggagc cagtcgacaa gcccaggact ga |

In some embodiments, the present disclosure relates to compositions and methods for gene transfer via a dual transposon and transposase system. Transposable elements are non-viral gene delivery vehicles found ubiquitously in nature. Transposon-based vectors have the capacity of stable genomic integration and long-lasting expression of transgene constructs in cells. Generally speaking, dual transposon and transposase systems work via a cut-and-paste mechanism whereby transposon DNA containing a transgene(s) of interest is integrated into chromosomal DNA by a transposase enzyme at a repetitive sequence site.

As would be appreciated in the art, a transposon often includes an open reading frame that encodes a transgene at the middle of transposon and terminal repeat sequences at the 5′ and 3′ end of the transposon. The translated transposase binds to the 5′ and 3′ sequence of the transposon and carries out the transposition function.

In embodiments, a transposon is used interchangeably with transposable elements, which are used to refer to polynucleotides capable of inserting copies of themselves into other polynucleotides. The term transposon is well known to those skilled in the art and includes classes of transposons that can be distinguished on the basis of sequence organization, for example short inverted repeats (ITRs) at each end, and/or directly repeated long terminal repeats (LTRs) at the ends. In some embodiments, the transposon as described herein may be described as a piggyBac like element, e.g. a transposon element that is characterized by its traceless excision, which recognizes TTAA sequence and restores the sequence at the insert site back to the original TTAA sequence after removal of the transposon.

In some embodiments, the non-viral vector is a transposon-mediated gene transfer system (e.g., a DNA plasmid transposon system) that is flanked by ITRs recognized by a transposase. In some embodiments, the ITRs flank the nucleic acid encoding the ABCA4 gene. The non-viral vector operates as a transposon-based vector system comprising a heterologous polynucleotide (also referred to as a transgene) flanked by two ends that are recognized by a transposase. The transposon ends include ITRs, which may be exact or inexact repeats and that are inverted in orientation with respect to each other. The transposase acts on the transposon ends to thereby “cut” the transposon (along with the transposon ends) from the vector and “paste,” or integrate, the transposon into a host genome. In embodiments, the transposase is provided as a DNA expression vector or as an expressible RNA or a protein such that long-term expression of the transposase does not occur in the transgenic cells.

In embodiments, a gene transfer system is a nucleic acid (DNA) encoding a transposon, and is referred to as a “donor DNA.” in embodiments, a nucleic acid encoding a transposase is helper RNA (Le. an mRNA encoding the transposase), and a nucleic acid encoding a transposon is donor DNA (or a DNA donor transposon). In embodiments, the donor DNA is incorporated into a plasmid. In embodiments, the donor DNA is a plasmid.

DNA donor transposons, which are mobile elements that use a “cut-and-paste” mechanism, include donor DNA that is flanked by two end sequences in the case of mammals (e.g. Myotis lucifugus, Myotis myotis, Pteropus vampyrus, Pipistrellus kuhlii, and Pan troglodytes) including humans (Homo sapiens), or Inverted Terminal Repeats (ITRs) in other living organisms such as insects (e.g. Trichoplusia ni) or amphibians (Xenopus species). Genomic DNA is excised by double strand cleavage at the hosts' donor site and the donor DNA is integrated at this site. A dual system that uses bioengineered transposons and transposases includes (1) a source of an active transposase that “cuts” at a specific nucleotide sequences such as TTAA and (2) DNA sequence(s) that are flanked by recognition end sequences or ITRs that are mobilized by the transposase. Mobilization of the DNA sequences permits the intervening nucleic acid, or a transgene, to be inserted at the specific nucleotide sequence (i.e. TTAA) without a DNA footprint.

In embodiments, a transposase is a Myotis lucifugus transposase (MLT, or MLT transposase), which comprises an amino acid sequence of SEQ ID NO: 10, or a variant having at least about 90%, or at least about 93%, or at least about 95%, or at least about 97%, or at least about 98%, or at least about 99% identity thereto. In embodiments, a transposase is a Myotis lucifugus transposase (MLT, or MLT transposase), which comprises an amino acid sequence of SEQ ID NO: 9, or a variant having at least about 90%, or at least about 93%, or at least about 95%, or at least about 97%, or at least about 98%, or at least about 99% identity thereto and S2X, wherein X is any amino acid or no amino acid, optionally X is A or G.

In embodiments, a transposase is a Myotis lucifugus transposase (MLT, or MLT transposase), which comprises an amino acid sequence of SEQ ID NO: 9, or a variant having at least about 90%, or at least about 93%, or at least about 95%, or at least about 97%, or at least about 98%, or at least about 99% identity thereto and S2X, wherein Xis any amino acid or no amino acid, optionally X is A or G and a C terminal deletions selected from L573X and E574X wherein X is no amino acid. In embodiments, the mutations are L573del, E574del, and S2A.

In embodiments, the MLT transposase comprises an amino acid sequence of SEQ ID NO: 10 with mutations L573del, E574del, and S2A:

(SEQ ID NO: 10)
MAQHSDYSDDEFCADKLSNYSCDSDLENASTSDEDSSDDEVMVRPRTLR
RRRISSSSSDSESDIEGGREEWSHVDNPPVLEDFLGHQGLNTDAVINNI
EDAVKLFIGDDFFEFLVEESNRYYNQNRNNFKLSKKSLKWKDITPQEMK
KFLGLIVLMGQVRKDRRDDYWTTEPWTETPYFGKTMTRDRFRQIWKAWH
FNNNADIVNESDRLCKVRPVLDYFVPKFINIYKPHQQLSLDEGIVPWRG
RLFFRVYNAGKIVKYGILVRLLCESDTGYICNMEIYCGEGKRLLETIQT
VVSPYTDSWYHIYMDNYYNSVANCEALMKNKFRICGTIRKNRGIPKDFQ
TISLKKGETKFIRKNDILLQVWQSKKPVYLISSIHSAEMEESQNIDRTS
KKKIVKPNALIDYNKHMKGVDRADQYLSYYSILRRTVKWTKRLAMYMIN
CALFNSYAVYKSVRQRKMGFKMFLKQTAIHWLTDDIPEDMDIVPDLQPV
PSTSGMRAKPPTSDPPCRLSMDMRKHTLQAIVGSGKKKNILRRCRVCSV
HKLRSETRYMCKFCNIPLHKGACFEKYHTLKNY,

or an amino acid sequence having at least about 90%, or at least about 93%, or at least about 95%, or at least about 97%, or at least about 98%, or at least about 99% identity thereto.

In some embodiments, an MLT transposase which comprises an amino acid sequence of SEQ ID NO: 10 is encoded by following nucleotide sequence:

(SEQ ID NO: 11)
atggcccagcacagcgactacagcgacgacgagttctgtgccgataagc
tgagtaactacagctgcgacagcgacctggaaaacgccagcacatccga
cgaggacagctctgacgacgaggtgatggtgcggcccagaaccctgaga
cggagaagaatcagcagctctagcagcgactctgaatccgacatcgagg
gcggccgggaagagtggagccacgtggacaaccctcctgttctggaaga
ttttctgggccatcagggcctgaacaccgacgccgtgatcaacaacatc
gaggatgccgtgaagctgttcataggagatgatttctttgagttcctgg
tcgaggaatccaaccgctattacaaccagaatagaaacaacttcaagct
gagcaagaaaagcctgaagtggaaggacatcacccctcaggagatgaaa
aagttcctgggactgatcgttctgatgggacaggtgcggaaggacagaa
gggatgattactggacaaccgaaccttggaccgagaccccttactttgg
caagaccatgaccagagacagattcagacagatctggaaagcctggcac
ttcaacaacaatgctgatatcgtgaacgagtctgatagactgtgtaaag
tgcggccagtgttggattacttcgtgcctaagttcatcaacatctataa
gcctcaccagcagctgagcctggatgaaggcatcgtgccctggcggggc
agactgttcttcagagtgtacaatgctggcaagatcgtcaaatacggca
tcctggtgcgccttctgtgcgagagcgatacaggctacatctgtaatat
ggaaatctactgcggcgagggcaaaagactgctggaaaccatccagacc
gtcgtttccccttataccgacagctggtaccacatctacatggacaact
actacaattctgtggccaactgcgaggccctgatgaagaacaagtttag
aatctgcggcacaatcagaaaaaacagaggcatccctaaggacttccag
accatctctctgaagaagggcgaaaccaagttcatcagaaagaacgaca
tcctgctccaagtgtggcagtccaagaaacccgtgtacctgatcagcag
catccatagcgccgagatggaagaaagccagaacatcgacagaacaagc
aagaagaagatcgtgaagcccaatgctctgatcgactacaacaagcaca
tgaaaggcgtggaccgggccgaccagtacctgtcttattactctatcct
gagaagaacagtgaaatggaccaagagactggccatgtacatgatcaat
tgcgccctgttcaacagctacgccgtgtacaagtccgtgcgacaaagaa
aaatgggattcaagatgttcctgaagcagacagccatccactggctgac
agacgacattcctgaggacatggacattgtgccagatctgcaacctgtg
cccagcacctctggtatgagagctaagcctcccaccagcgatcctccat
gtagactgagcatggacatgcggaagcacaccctgcaggccatcgtcgg
cagcggcaagaagaagaacatccttagacggtgcagggtgtgcagcgtg
cacaagctgcggagcgagactcggtacatgtgcaagttttgcaacattc
ccctgcacaagggagcctgcttcgagaagtaccacaccctgaagaatta
ctag,

or a nucleotide sequence having at least about 90%, or at least about 93%, or at least about 95%, or at least about 97%, or at least about 98%, or at least about 99% identity thereto.

In some embodiments, the MLT transposase (e.g., the MLT transposase having an amino acid sequence of SEQ ID NO: 10, or an amino add sequence having at least about 90%, or at least about 93%, or at least about 95%, or at least about 97%, or at least about 98%, or at least about 99% identity thereto) comprises one or more hyperactive mutations that confer hyperactivity upon the MLT transposase. In embodiments, the hyperactive mutations, relative to the amino add sequence of SEQ ID NO: 10 or a functional equivalent thereof, are one or more of S8X, C13X, and N125X mutations, wherein X is optionally any amino add or no amino add, optionally X is P, R, or K. In embodiments, the mutations are S8P, C13R, and N125K. In some embodiments, the MLT transposase has S8P and G13R mutations. In some embodiments, the MLT transposase has N125K mutation. In some embodiments, the MLT transposase has all three S8P, C13R, and N125K mutations.

In some embodiments, an MLT transposase is encoded by a nucleotide sequence (SEQ ID NO: 12) that corresponds to an amino acid (SEQ ID NO: 13) having the N125K mutation relative to the amino acid sequence of SEQ ID NO: 10 or a functional equivalent thereof, wherein SEQ ID NO: 12 and SEQ ID NO: 13 are as follows:

| (SEQ ID NO: 12) | |
|---|---|
| 1 | atggcccagc acagcgacta cagcgacgac gagttctgtg ccgataagct gagtaactac |
| 61 | agctgcgaca gcgacctgga aaacgccagc acatccgacg aggacagctc tgacgacgag |
| 121 | gtgatggtgc ggcccagaac cctgagacgg agaagaatca gcagctctag cagcgactct |
| 181 | gaatccgaca tcgagggcgg ccgggaagag tggagccacg tggacaaccc tcctgttctg |
| 241 | gaagattttc tgggccatca gggcctgaac accgacgccg tgatcaacaa catcgaggat |
| 301 | gccgtgaagc tgttcatagg agatgatttc tttgagttcc tggtcgagga atccaaccgc |
| 361 | tattacaacc agaagagaaa caacttcaag ctgagcaaga aaagcctgaa gtggaaggac |
| 421 | atcacccctc aggagatgaa aaagttcctg ggactgatcg ttctgatggg acaggtgcgg |
| 481 | aaggacagaa gggatgatta ctggacaacc gaaccttgga ccgagacccc ttactttggc |
| 541 | aagaccatga ccagagacag attcagacag atctggaaag cctggcactt caacaacaat |
| 601 | gctgatatcg tgaacgagtc tgatagactg tgtaaagtgc ggccagtgtt ggattacttc |
| 661 | gtgcctaagt tcatcaacat ctataagcct caccagcagc tgagcctgga tgaaggcatc |
| 721 | gtgccctggc ggggcagact gtccttcaga gtgtacaatg ctggcaagat cgtcaaatac |
| 781 | ggcatcctgg tgcgccttct gtgcgagagc gatacaggct acatctgtaa tatggaaatc |
| 841 | tactgcggcg agggcaaaag actgctggaa accatccaga ccgtcgtttc cccttatacc |
| 901 | gacagctggt accacatcta catggacaac tactacaatt ctgtggccaa ctgcgaggcc |
| 961 | ctgatgaaga acaagtttag aatctgcggc acaatcagaa aaaacagagg catccctaag |
| 1021 | gacttccaga ccatctctct gaagaagggc gaaaccaagt tcatcagaaa gaacgacatc |
| 1081 | ctgctccaag tgtggcagtc caagaaaccc gtgtacctga tcagcagcat ccatagcgcc |
| 1141 | gagatggaag aaagccagaa catcgacaga acaagcaaga agaagatcgt gaagcccaat |
| 1201 | gctctgatcg actacaacaa gcacatgaaa ggcgtggacc gggccgatca gtacctgtct |
| 1261 | tattactcta tcctgagaag aacagtgaaa tggaccaaga gactggccat gtacatgatc |
| 1321 | aattgcgccc tgttcaacag ctacgccgtg tacaagtccg tgcgacaaag aaaaatggga |
| 1381 | ttcaagatgt tcctgaagca gacagccatc cactggctga cagacgacat tcctgaggac |
| 1441 | atggacattg tgccagatct gcaacctgtg cccagcacct ccggtatgag agctaagcct |
| 1501 | cccaccagcg atcctccatg tagactgagc atggacatgc ggaagcacac cctgcaggcc |
| 1561 | atcgtcggca gcggcaagaa gaagaacatc cttagacggt gcagggtgtg cagcgtgcac |
| 1621 | aagctgcgga gcgagactcg gtacatgtgc aagttttgca acattcccct gcacaaggga |
| 1681 | gcctgcttcg agaagtacca caccctgaag aattactag, |

or a nucleotide sequence having at least about 90%, or at least about 93%, or at least about 95%, or at least about 97%, or at least about 98%, or at least about 99% identity thereto (the codon corresponding to the N125K mutation is underlined and bolded).

| (SEQ ID NO: 13) | |
|---|---|
| 1 | MAQHSDYSDD EFCADKLSNY SCDSDLENAS TSDEDSSDDE VMVRPRTLRR RRISSSSSDS |
| 61 | ESDIEGGREE WSHVDNPPVL EDFLGHQGLN TDAVINNIED AVKLFIGDDF FEFLVEESNR |
| 121 | YYNQKRNNFK LSKKSLKWKD ITPQEMKKFL GLIVLMGQVR KDRRDDYWTT EPWTETPYFG |
| 181 | KTMTRDRFRQ IWKAWHFNNN ADIVNESDRL CKVRPVLDYF VPKFINIYKP HQQLSLDEGI |
| 241 | VPWRGRLFFR VYNAGKIVKY GILVRLLCES DTGYICNMEI YCGEGKRLLE TIQTVVSPYT |
| 301 | DSWYHIYMDN YYNSVANCEA LMKNKFRICG TIRKNRGIPK DFQTISLKKG ETKFIRKNDI |
| 361 | LLQVWQSKKP VYLISSIHSA EMEESQNIDR TSKKKIVKPN ALIDYNKHMK GVDRADQYLS |
| 421 | YYSILRRTVK WTKRLAMYMI NCALFNSYAV YKSVRQRKMG FKMFLKQTAI HWLTDDIPED |
| 481 | MDIVPDLQPV PSTSGMRAKP PTSDPPCRLS MDMRKHTLQA IVGSGKKKNI LRRCRVCSVH |
| 541 | KLRSETRYMC KFCNIPLHKG ACFEKYHTLK NY, |

or an amino acid sequence having at least about 90%, or at least about 93%, or at least about 95%, or at least about 97%, or at least about 98%, or at least about 99% identity thereto (the amino acid corresponding to the N125K mutation is underlined and bolded).

In some embodiments, the MLT transposase encoded by the nucleotide sequence of SEQ ID NO: 12 and having the amino acid sequence of SEQ ID NO: 13 is referred to as an MLT transposase 1 (or MLT 1).

In some embodiments, an MLT transposase encoded by a nucleotide sequence (SEQ ID NO: 14) that corresponds to an amino acid (SEQ ID NO: 15) having the S8P and C13R mutations relative to the amino acid sequence of SEQ ID NO: 10 or a functional equivalent thereof, wherein SEQ ID NO: 14 and SEQ ID NO: 15 are as follows:

| (SEQ ID NO: 14) | |
|---|---|
| 1 | atggcccagc acagcgacta ccccgacgac gagttcagac ccgataagct gagtaactac |
| 61 | agctgcgaca gcgacctgga aaacgccagc acatccgacg aggacagctc tgacgacgag |
| 121 | gtgatggtgc ggcccagaac cctgagacgg agaagaatca gcagctctag cagcgactct |
| 181 | gaatccgaca tcgagggcgg ccgggaagag tggagccacg tggacaaccc tcctgttctg |
| 241 | gaagattttc tgggccatca gggcctgaac accgacgccg tgatcaacaa catcgaggat |
| 301 | gccgtgaagc tgttcatagg agatgatttc tttgagttcc tggtcgagga atccaaccgc |
| 361 | tattacaacc agaatagaaa caacttcaag ctgagcaaga aaagcctgaa gtggaaggac |
| 421 | atcacccctc aggagatgaa aaagttcctg ggactgatcg ttctgatggg acaggtgcgg |
| 481 | aaggacagaa gggatgatta ctggacaacc gaaccttgga ccgagacccc ttactttggc |
| 541 | aagaccatga ccagagacag attcagacag atctggaaag cctggcactt caacaacaat |
| 601 | gctgatatcg tgaacgagtc tgatagactg tgtaaagtgc ggccagtgtt ggattacttc |
| 661 | gtgcctaagt tcatcaacat ctataagcct caccagcagc tgagcctgga tgaaggcatc |
| 721 | gtgccctggc ggggcagact gttcttcaga gtgtacaatg ctggcaagat cgtcaaatac |
| 781 | ggcatcctgg tgcgccttct gtgcgagagc gatacaggct acatctgtaa tatggaaatc |
| 841 | tactgcggcg agggcaaaag actgctggaa accatccaga ccgtcgtttc cccttatacc |
| 901 | gacagctggt accacatcta catggacaac tactacaatt ctgtggccaa ctgcgaggcc |
| 961 | ctgatgaaga acaagtttag aatctgcggc acaatcagaa aaaacagagg catccctaag |
| 1021 | gacttccaga ccatctctct gaagaagggc gaaaccaagt tcatcagaaa gaacgacatc |
| 1081 | ctgctccaag tgtggcagtc caagaaaccc gtgtacctga tcagcagcat ccatagcgcc |
| 1141 | gagatggaag aaagccagaa catcgacaga acaagcaaga agaagatcgt gaagcccaat |
| 1201 | gctctgatcg actacaacaa gcacatgaaa ggcgtggacc gggccgacca gtacctgtct |
| 1261 | tattactcta tcctgagaag aacagtgaaa tggaccaaga gactggccat gtacatgatc |
| 1321 | aattgcgccc tgttcaacag ctacgccgtg tacaagtccg tgcgacaaag aaaaatggga |
| 1381 | ttcaagatgt tcctgaagca gacagccatc cactggctga cagacgacat tcctgaggac |
| 1441 | atggacattg tgccagatct gcaacctgtg cccagcacct ctggtatgag agctaagcct |
| 1501 | cccaccagcg atcctccatg tagactgagc atggacatgc ggaagcacac cctgcaggcc |
| 1561 | atagtcggca gcggcaagaa gaagaacatc cttagacggt gcagggtgtg cagcgtgcac |
| 1621 | aagctgcgga gcgagactcg gtacatgtgc aagttttgca acattcccct gcacaaggga |
| 1681 | gcctgcttcg agaagtacca caccctgaag aattactag, |

or a nucleotide sequence having at least about 90%, or at least about 93%, or at least about 95%, or at least about 97%, or at least about 98%, or at least about 99% identity thereto (the codons corresponding to the S8P and C13R mutations are underlined and bolded).

| (SEQ ID NO: 15) | |
|---|---|
| 1 | MAQHSDYPDD EFRADKLSNY SCDSDLENAS TSDEDSSDDE VMVRPRTLRR RRISSSSSDS |
| 61 | ESDIEGGREE WSHVDNPPVL EDFLGHQGLN TDAVINNIED AVKLFIGDDF FEFLVEESNR |
| 121 | YYNQNRNNFK LSKKSLKWKD ITPQEMKKFL GLIVLMGQVR KDRRDDYWTT EPWTETPYFG |
| 181 | KTMTRDRFRQ IWKAWHFNNN ADIVNESDRL CKVRPVLDYF VPKFINIYKP HQQLSLDEGI |
| 241 | VPWRGRLFFR VYNAGKIVKY GILVRLLCES DTGYICNMEI YCGEGKRLLE TIQTVVSPYT |
| 301 | DSWYHIYMDN YYNSVANCEA LMKNKFRICG TIRKNRGIPK DFQTISLKKG ETKFIRKNDI |
| 361 | LLQVWQSKKP VYLISSIHSA EMEESQNIDR TSKKKIVKPN ALIDYNKHMK GVDRADQYLS |
| 421 | YYSILRRTVK WTKRLAMYMI NCALFNSYAV YKSVRQRKMG FKMFLKQTAI HWLTDDIPED |
| 481 | MDIVPDLQPV PSTSGMRAKP PTSDPPCRLS MDMRKHTLQA IVGSGKKKNI LRRCRVCSVH |
| 541 | KLRSETRYMC KECNIPLHKG ACFEKYHTLK NY, |

or an amino acid sequence having at least about 90%, or at least about 93%, or at least about 95%, or at least about 97%, or at least about 98%, or at least about 99% identity thereto (the amino acids corresponding to the S8P and C13R mutations are underlined and bolded).

In some embodiments, the MLT transposase encoded by the nucleotide sequence of SEQ ID NO: 14 and having the amino acid sequence of SEQ ID NO: 15 is referred to as an MLT transposase 2 (or MLT 2).

In some embodiments, the transposase is from a Tc1/mariner transposon system. See, e,g, Plasterk et al. Trends in Genetics. 1999; 15 (8): 32S-32.

In some embodiments, the transposase is from a Sleeping Beauty transposon system (see, e.g. Cell. 1997;91:501-510) or a piggyBac transposon system (see, e.g. Trends Biotechnol. 2015 September; 33(9):525-33. doi: 10.1016/j.tibtech.2015.06.009. Epub 2015 Jul. 23).

In some embodiments, the transposase is from a LEAP-IN 1 type or LEAP-IN transposon system (Biotechnol J. 2018 October; 13(10):e1700748, doi: 10.1002/biot.201700748. Epub 2018 Jun. 11).

In some embodiments, a non-viral vector includes a LEAP-IN 1 type of LEAPIN Transposase (ATM, Newark, Calif.), The LEAPIN Transposase system includes a transposase (e.g., a transposase mRNA) and a vector containing one or more genes of interest (transposons), selection markers, regulatory elements, etc., flanked by the transposon cognate inverted terminal repeats (ITRs) and the transposition recognition motif (TTAT). Upon co-transfection of vector DNA and transposase mRNA, the transiently expressed enzyme catalyzes high-efficiency and precise integration of a single copy of the transposon cassette (all sequences between the ITRs) at one or more sites across the genome of the host cell. Hottentot et al. In Genotyping: Methods and Protocols. White S J, Cantsilieris S, eds: 185-196. (New York, N.Y.: Springer): 2017. pp. 185-196. The LEAPIN Transposase generates stable transgene integrants with various advantageous characteristics, including single copy integrations at multiple genomic loci, primarily in open chromatin segments; no payload limit, so multiple independent transcriptional units may be expressed from a single construct; the integrated transgenes maintain their structural and functional integrity; arid maintenance of transgene integrity ensures the desired chain ratio in every recombinant cell.

In some embodiments, the ABCA4 is operably coupled to a promoter that can influence overall expression levels and cell-specificity of the transgenes (e.g. ABCA4 or a functional fragment thereof).

In some embodiments, the promoter is a CAG promoter (cytomegalovirus (CMV) enhancer fused to the chicken -actin promoter and rabbit beta-Globin splice acceptor) (1732 bp), which expresses in both RPE and photoreceptor levels in vivo and in vitro. In some embodiments, the CAG promoter comprises the following nucleotide sequence (SEQ ID NO: 16):

| (SEQ ID NO: 16) | |
|---|---|
| 1 | tcgacattga ttattgacta gttattaata gtaatcaatt acggggtcat tagttcatag |
| 61 | cccatatatg gagttccgcg ttacataact tacggtaaat ggcccgcctg gctgaccgcc |
| 121 | caacgacccc cgcccattga cgtcaataat gacgtatgtt cccatagtaa cgccaatagg |
| 181 | gactttccat tgacgtcaat gggtggagta tttacggtaa actgcccact tggcagtaca |
| 241 | tcaagtgtat catatgccaa gtacgccccc tattgacgtc aatgacggta aatggcccgc |
| 301 | ctggcattat gcccagtaca tgaccttatg ggactttcct acttggcagt acatctacgt |
| 361 | attagtcatc gctattacca tggtcgaggt gagccccacg ttctgcttca ctctccccat |
| 421 | ctcccccccc tccccacccc caattttgta tttatttatt ttttaattat tttgtgcagc |
| 481 | gatgggggcg gggggggggg gggggcgcgc gccaggcggg gcggggcggg gcgaggggcg |
| 541 | gggcggggcg aggcggagag gtgcggcggc agccaatcag agcggcgcgc tccgaaagtt |
| 601 | tccttttatg gcgaggcggc ggcggcggcg gccctataaa aagcgaagcg cgcggcgggc |
| 661 | gggagtcgct gcgcgctgcc ttcgccccgt accccactcc gccgccgcct cgcgccgccc |
| 721 | gccccggctc tgactgaccg cgttactccc acaggtgagc gggcgggacg gcccttctcc |
| 781 | tccgggctgt aattagcgct tggtttaatg acggcttgtt tcttttctgt ggctgcgtga |
| 341 | aagccttgag gggctccggg agggcccttt gtgcgggggg agcggctcgg ggggtgcgtg |
| 901 | cgtgtgtgtg tgcgtgggga gcgccgcgtg cggctccgcg ctgcccggcg gctgtgagcg |
| 961 | ctgcgggcgc ggcgcggggc tttgtgcgct ccgcagtgtg cgcgagggga gcgcggccgg |
| 1021 | gggcggtgcc ccgcggtgcg gggggggctg cgaggggaac aaaggctgcg tgcggggtgt |
| 1081 | gtgcgtgggg gggtgagcag ggggtgtggg cgcgtcggtc gggctgcaac cccccctgca |
| 1141 | cccccctccc cgagttgctg agcacggccc ggcttcgggt gcggggctcc gtacggggcg |
| 1201 | tggcgcgggg ctcgccgtgc cgggcggggg gtggcggcag gtgggggtgc cgggcggggc |
| 1261 | ggggccgcct cgggccgggg agggctcggg ggaggggcgc ggcggccccc ggagcgccgg |
| 1321 | cggctgtcga ggcgcggcga gccgcagcca ttgcctttta tggtaatcgt gcgagagggc |
| 1381 | gcagggactt cctttgtccc aaatctgtgc ggagccgaaa tctgggaggc gccgccgcac |
| 1441 | cccctctagc gggcgcgggg cgaagcggtg cggcgccggc aggaaggaaa tgggcgggga |
| 1501 | gggccttcgt gcgtcgccgc gccgccgtcc ccttctccct ctccagcctc ggggctgtcc |
| 1561 | gcggggggac ggctgccttc gggggggacg gggcagggcg gggttcggct tctggcgtgt |
| 1621 | gaccggcggc tctagagcct ctgctaacca tgttcatgcc ttcttctttt tcctacagct |
| 1681 | cctgggcaac gtgctggtta ttgtgctgtc tcatcatttt ggcaaagaat tc, |

or a variant having at least about 80%, or at least about 85%, or at least about 90%, or at least about 93%, or at least about 95%, or at least about 97%, or at least about 98% identity thereto.

In some embodiments, the promoter is CMV enhancer, chicken beta-Actin promoter and rabbit beta-Globin splice acceptor site (CAG), optionally comprising a nucleic acid sequence of SEQ ID NO: 16, or a variant having at least about 50%, or at least about 60%, or at least about 70%, or at least about 80%, or at least about 85%, or of at least about 90%, or at least about 93%, or at least about 95%, or at least about 97%, or at least about 98% identity thereto.

In some embodiments, the promoter is tissue-specific, i.e. retina-specific promoter. In embodiments in which the transposase is a DNA sequence encoding the transposase, such DNA sequence is also operably linked to a promoter. A variety of promoters can be used, including tissue-specific promoters, inducible promoters, constitutive promoters, etc.

In some embodiments, the retina-specific promoter is a retinal pigment epithelium (RPE) promoter, which can be RPE65 (retinal pigment epithelium-specific 65 kDa protein gene), IRBP (interphotoreceptor retinoid-binding protein), or VMD2 (vitelliform macular dystrophy 2) promoter.

The RPE65, IRBP, and VMD2 promoters are described in, e.g., Aguirre: Invest Ophthalmol Vis Sci. 2017; 58(12):5399-5411. doi:10.1167/iovs.17-22978. An example of an RPE65 promoter that can be used in some embodiments is:

| (SEQ ID NO: 3) | |
|---|---|
| 1 | GATCCAACAA AAGTGATTAT ACCCCCCAAA ATATGATGGT AGTATCTTAT ACTACCATCA |
| 61 | TTTTATAGGC ATAGGGCTCT TAGCTGCAAA TAATGGAACT AACTCTAATA AAGCAGAACG |
| 121 | CAAATATTGT AAATATTAGA GAGCTAACAA TCTCTGGGAT GGCTAAAGGA TGGAGCTTGG |
| 181 | AGGCTACCCA GCCAGTAACA ATATTCCGGG CTCCACTGTT GAATGGAGAC ACTACAACTG |
| 241 | CCTTGGATGG GCAGAGATAT TATGGATGCT AAGCCCCAGG TGCTACCATT AGGACTTCTA |
| 301 | CCACTGTCCT AACGGGTGGA GCCCATCACA TGCCTATGCC CTCACTGTAA GGAAATGAAG |
| 361 | CTACTGTTGT ATATCTTGGG AAGCACTTGG ATTAATTGTT ATACAGTTTT GTTGAAGAAG |
| 421 | ACCCCTAGGG TAAGTAGCCA TAACTGCACA CTAAATTTAA AATTGTTAAT GAGTTTCTCA |
| 481 | AAAAAAATGT TAAGGTTGTT AGCTGGTATA GTATATATCT TGCCTGTTTT CCAAGGACTT |
| 541 | CTTTGGGCAG TACCTTGTCT GTGCTGGCAA GCAACTGAGA CTTAATGAAA GAGTATTGGA |
| 601 | GATATGAATG AATTGATGCT GTATACTCTC AGAGTGCCAA ACATATACCA ATGGACAAGA |
| 661 | AGGTGAGGCA GAGAGCAGAC AGGCATTAGT GACAAGCAAA GATATGCAGA ATTTCATTCT |
| 721 | CAGCAAATGA AAAGTCCTCA ACCTGGTTGG AAGAATATTG GCACTGAATG GTATCAATAA |
| 781 | GGTTGCTAGA GAGGGTTAGA GGTGCACAAT GTGCTTCCAT AACATTTTAT ACTTCTCCAA |
| 841 | TCTTAGCACT AATCAAACAT GGTTGAATAC TTTGTTTTCT ATAACTCTTA CAGAGTTATA |
| 901 | AGATCTGTGA AGACAGGGAC AGGGACAATA CCCATCTCTG TCTGGTTCAT AGGTGGTATG |
| 961 | TAATAGATAT TTTTAAAAAT AAGTGAGTTA ATGAATGAGG GTGAGAATGA AGGCACAGAG |
| 1021 | GTATTAGGGG GAGGTGGGCC CCAGAGAATG GTGCCAAGGT CCAGTGGGGT GACTGGGATC |
| 1081 | AGCTCAGGCC TGACGCTGGC CACTCCCACC TAGCTCCTTT CTTTCTAATC TGTTCTCATT |
| 1141 | CTCCTTGGGA AGGATTGAGG TCTCTGGAAA ACAGCCAAAC AACTGTTATG GGAACAGCAA |
| 1201 | GCCCAAATAA AGCCAAGCAT CAGGGGGATC TGAGAGCTGA AAGCAACTTC TGTTCCCCCT |
| 1261 | CCCTCAGCTG AAGGGGTGGG GAAGGGCTCC CAAAGCCATA ACTCCTTTTA AGGGATTTAG |
| 1321 | AAGGCATAAA AAGGCCCCTG GCTGAGAACT TCCTTCTTCA TTCTGCAGTT GGTGCCAGAA |
| 1381 | CTCTGGATCC TGAACTGGAA GAAA, |

or a functional fragment of a variant having at least about 50%, or at least about 60%, or at least about 70%, or at least about 80%, or at least about 85%, or at least about 90%, or at least about 93%, or at least about 95%, or at least about 97%, or at least about 98% identity thereto.

A human interphotoreceptor retinoid-binding protein (IRBP) promoter has been demonstrated to rescue photoreceptors from progressive degeneration. al-Ubaldi & Baehr. J. Cell Biol. 1992; 119: 1681-1687, An example of an IRBP promoter (1325 bp) that can be used in some embodiments (adapted from Bobola et al., J. Biol. Chem. 1995; 270:1289-1294) is;

| (SEQ ID NO: 4) | |
|---|---|
| 1 | gatgcctact gaggcacaca ggggagcctg cctgctgccc gctcagccaa ggcggtgttg |
| 61 | ctggagccag cttgggacag ctctcccaac gctctgccct ggccttgcga cccactctct |
| 121 | gggccgtagt tgtctgtctg ttaagtgagg aaagtgccca tctccagagg cattcagcgg |
| 181 | caaagcaggg cttccaggtt ccgaccccat agcaggactt cttggatttc tacagccagt |
| 241 | cagttgcaag cagcacccat attatttcta taagaagtgg caggagctgg atctgaagag |
| 301 | tcagcagtct acctttccct gtttcttgtg ctttatgcag tcaggaggaa tgatctggat |
| 361 | tccatgtgaa gcctgggacc acggagaccc aagacttcct gcttgattct ccctgcgaac |
| 421 | tgcaggctgt gggctgagcc ttcaagaagc aggagtcccc tctagccatt aactctcaga |
| 481 | gctaacctca tttgaatggg aacactagtc ctgtgatgtc tggaaggtgg gcgcctctac |
| 541 | actccacacc ctacatggtg gtccagacac atcattccca gcattagaaa gctctagggg |
| 601 | gacccgttct gttccctgag gcattaaagg gacatagaaa taaatctcaa gctctgaggc |
| 661 | tgatgccagc ctcagactca gcctctgcac tgtatgggcc aattgtagcc ccaaggactt |
| 721 | cttcttgctg caccccctat ctgtccacac ctaaaacgat gggcttctat tagttacaga |
| 781 | actctctggc ctgttttgtt ttgctttgct ttgttttgtt ttgttttttt gtttttttgt |
| 841 | tttttagcta tgaaacagag gtaatatcta atacagataa cttaccagta atgagtgctt |
| 901 | cctacttact gggtactggg aagaagtgct ttacacatat tttctcattt aatctacaca |
| 961 | ataagtaatt aagacatttc cctgaggcca cgggagagac agtggcagaa cagttctcca |
| 1021 | aggaggactt gcaagttaat aactggactt tgcaaggctc tggtggaaac tgtcagcttg |
| 1081 | taaaggatgg agcacagtgt ctggcatgta gcaggaacta aaataatggc agtgattaat |
| 1141 | gttatgatat gcagacacaa cacagcaaga taagatgcaa tgtaccttct gggtcaaacc |
| 1201 | accctggcca ctcctccccg atacccaggg ttgatgtgct tgaattagac aggattaaag |
| 1261 | gcttactgga gctggaagcc ttgccccaac tcaggagttt agccccagcc cttctgtcca |
| 1321 | ccagc, |

or a functional fragment of a variant having at least about 50%, or at least about 60%, or at least about 70%, or at least about 80%, or at least about 85%, or at least about 90%, or at least about 93%, or at least about 95%, or at least about 97%, or at least about 98% identity thereto.

A human VMD2 promoter was shown to specifically and exclusively target transgene expression to the RPE cells in vivo after a single subretinal injection (in dogs). See Guziewicz et al., PloS One vol. 8,10 e75666. 15 Oct. 2013, doi:10.1371/journal.pone.0075666. An example of a VMD2 promoter sequence (624 bp) that is the upstream region of the BEST1 gene (see Esumi et al., J. Biol. Chem. 2004; 279(18)19064-19073), which can be used in some embodiments, is:

| (SEQ ID NO: 5) | |
|---|---|
| 1 | aattctgtca ttttactagg gtgatgaaat tcccaagcaa caccatcctt ttcagataag |
| 61 | ggcactgagg ctgagagagg agctgaaacc tacccggggt caccacacac aggtggcaag |
| 121 | gctgggacca gaaaccagga ctgttgactg cagcccggta ttcattcttt ccatagccca |
| 181 | cagggctgtc aaagacccca gggcctagtc agaggctcct ccttcctgga gagttcctgg |
| 241 | cacagaagtt gaagctcagc acagccccct aacccccaac tctctctgca aggcctcagg |
| 301 | ggtcagaaca ctggtggagc agatccttta gcctctggat tttagggcca tggtagaggg |
| 361 | ggtgttgccc taaattccag ccctggtctc agcccaacac cctccaagaa gaaattagag |
| 421 | gggccatggc caggctgtgc tagccgttgc ttctgagcag attacaagaa gggactaaga |
| 481 | caaggactcc tttgtggagg tcctggctta gggagtcaag tgacggcggc tcagcactca |
| 541 | cgtgggcagt gccagcctct aagagtgggc aggggcactg gccacagagt cccagggagt |
| 601 | cccaccagcc tagtcgccag acct, |

or a functional fragment of a variant having at least about 50%, or at least about 60%, or at least about 70%, or at least about 80%, or at least about 85%, or at least about 90%, or at least about 93%, or at least about 95%, or at least about 97%, or at least about 98% identity thereto.

In some embodiments, the retina-specific promoter is a photoreceptor promoter, optionally selected from β-phosphodiesterase (PDE), rhodopsin kinase (GRK1), CAR (cone arrestin), retinitis pigmentosa 1 (RP1), and L-opsin. The PDE and RP1 promoters, as well as a rhodopsin (Rho) promoter, were shown to drive photoreceptor-specific expression in vitro. Kan et al., Molecular Therapy, vol. 15, Suppl. 1, S258, May 1, 2007. An example of a PDE promoter (200 bp) that can be used in some embodiments (e.g., as described in Di Polo et al., Nucleic Acids Res. 1997; 25(19):3863-3867) is:

| (SEQ ID NO: 6) | |
|---|---|
| 1 | acgcctgcaa caggcaggag atcccccaac agttactccc agccttcatt ccacagggtc |
| 61 | tggttttcct ggaggtggga agtcccaggg tctgaggaga gggagcgcag gcccccattt |
| 121 | gtaggagtga gtcagctgac ccgcccccgg ggttcctaat ctcactaaga aagactttgc |
| 181 | tgatgacagg gtttcctggg, |

or a functional fragment of a variant having at least about 50%, or at least about 60%, or at least about 70%, or at least about 80%, or at least about 85%, or at least about 90%, or at least about 93%, or at least about 95%, or at least about 97%, or at least about 98% identity thereto.

The human rhodopsin kinase (GRK1) gene promoter was shown to be active and specific for rod and cone photoreceptors, and, because of its small size and proven activity in cones, it is a promoter of choice for somatic gene transfer and gene therapy targeting rods and cones. Khani et al., Investigative Ophthalmology & Visual Science September 2007; vol.48:3954-3961. An example of a GRK1 promoter (295 bp) that can be used in some embodiments (see Khani et al, 2007; McDougald et al., Mol Ther Methods Clin Dev. 2019; 13:380-389. Published 2019 Mar. 28) is:

| (SEQ ID NO: 7) | |
|---|---|
| 1 | gggccccaga agcctggtgg ttgtttgtcc ttctcagggg aaaagtgagg cggccccttg |
| 61 | gaggaagggg ccgggcagaa tgatctaatc ggattccaag cagctcaggg gattgtcttt |
| 121 | ttctagcacc ttcttgccac tcctaagcgt cctcagtgac cccggctggg atttagcctg |
| 181 | gtgctgtgtc agccccggtc tcccaggggc ttcccagtgg tccccaggaa ccctcgacag |
| 241 | ggccagggcg tctctctcgt ccagcaaggg cagggacggg ccacaggcca agggc, |

or a functional fragment of a variant having at least about 50%, or at least about 60%, or at least about 70%, or at least about 80%, or at least about 85%, or at least about 90%, or at least about 93%, or at least about 95%, or at least about 97%, or at least about 98% identity thereto.

CAR promoters were also shown to drive strong expression in retina. Dyka et al., Adv Exp Med Biol. 2014; 801:695-701. In some embodiments, a CAR promoter (2026 bp) (see McDougald et al., Mol Ther Methods Clin Dev. 2019; 13:380-389. Published 2019 Mar. 28) is:

| (SEQ ID NO: 8) | |
|---|---|
| 1 | ctggtgatta cattagggcc cacctggata atccagaatg atctccctat ttcaacatcc |
| 61 | ttaatttatt cacatctgca aagtctcttt ttcatataag gtaatgttca tcggttccca |
| 121 | ggattaagac ctgacatctt tgggggcata attcagcttg ccacagtagg taaaaattca |
| 181 | ttgagctgca gttaagattt gtgaatttta cctcagtcaa gaaatgcaca aacttctgga |
| 241 | aaagagtaat gatttacatt ccatcataat aatgaattaa agacctagca gatctactct |
| 301 | tttcctaccg agaggcccat ggatctgagt agaaagagaa gataagcggg attgagtacc |
| 361 | taaaagggag gtaggagact cgagtgtggg tctaaagaca aaaacaggct gaccactagt |
| 421 | cattctagag atctgggaaa ggtttcctga atgatgaaaa taagcataca agaagagagg |
| 481 | ccttcctttc ctgccattga atattgccat gtctggcatg aaaagtagat tcattctgac |
| 541 | ttttcgcctt cctcgcagac accaaccttg gcatgtatac aaatctttcc tgtatgtcca |
| 601 | gcatcagttc ctatcccact gtggtacctg cagaatctgg gcttcttgca ctatctgaaa |
| 661 | gcccctgaga aggagagagt tatagtaact aaacaaccag gccctgagat gcatattggc |
| 721 | taggaatggc aggggctgac actgtgaact gtgcaaagag aatatgggac agctgtccag |
| 781 | ggccctcagt gaggggcagg agttagggaa ggccctgccc agccctctga gccatagcca |
| 841 | tagccatcct ctgaggaatg gacaccccat tgtgggggtt ggggttgagg gctgtgtcta |
| 901 | tagataacta ctaatgtcca gactgctgta aggggaggtg aaggaggtca gagtcctgaa |
| 961 | accccagagc ttatagattc tgtctctaca ttttctatgc ccgtgaagcc tgagcctagg |
| 1021 | ccctgtggga aggacagtca agaaaggaag attactttgt tgttgctgtt gtgggggtcc |
| 1081 | tggcagctga agagacagaa atatctctaa ttccatgagc ggtcatacga ggcaagagaa |
| 1141 | gctgcttaga gcatggactt agttagtttc agggattgga cagagtcaag agctggggtg |
| 1201 | aggaggttta ccctcggtag gggtgacaca gatgtcaacc gcctattccc tccacatgca |
| 1261 | tgtcctgcca gaagaacctg tccctgggct gggaatctta tattaccttc ctctccaatg |
| 1321 | agaagagaag ttcaaggctc acagacatgt gcatacacag ctcaatgcac tcagatcccc |
| 1381 | ctccaccact cctgccccca ctacctacag gagattgact cctgctgtgc acataagctg |
| 1441 | ggataatcag ggtttctaaa catcagcttc aaaagtccaa tgtcccaaag tggtgggggg |
| 1501 | ctggggacga ggtactcttt cccataccct tggcttttgt gtggcctgga gccgctgata |
| 1561 | tagagattgg agtgggacac gaggtattcc tttcaaaaac acaaaggcct atactttgag |
| 1621 | ccctcccatt tcaatccccc accatgcttc acctttaaga cctccaactc cactttgatc |
| 1681 | ccagttctca ggttcaaggc ctcacaaggc caaaatcctg aagttaccct tctcaaactc |
| 1741 | ccttgccttt aacatcatca gaatcaacct cctaccccca ctctgtccca gcagcaatag |
| 1801 | cctgctaatc ttttagccac taatctttta ggcactaatc tgctttccaa actcttggca |
| 1861 | cctgaactat ttatagcagt gttttatgcc cccccaccaa gaaccctatt cttttcccat |
| 1921 | gacccccacc aatcaaaaca ctcagaggac tgtgggtata agaggctggg gaggcaggca |
| 1981 | tagcaaccag agctggagac tgatgtgaac ttcatctctc tcccca, |

or a functional fragment of a variant having at least about 50%, or at least about 60%, or at least about 70%, or at least 15 about 80%, or at least about 85%, or at least about 90%, or at least about 93%, or at least about 95%, or at least about 97%, or at least about 98% identity thereto.

A human L-opsin promoter was shown to direct high-level GFP expression in mouse photoreceptors. Ye et al., Hum Gene Ther. 2016; 27(1):72-82. In some embodiments, L-opsin promoter (1726 bp) (see Lee et al., Vision Res. 2008 February; 48(3):332-8) is:

| (SEQ ID NO: 9) | |
|---|---|
| 1 | gaggctgagg ggtggggaaa gggcatgggt gtttcatgag gacagagctt ccgtttcatg |
| 61 | caatgaaaag agtttggaga aggatggtgg tgactggact atacacttac acacggtagc |
| 121 | gatggtacac tttgtattat gtatatttta ccacgatctt tttaaagtgt caaaggcaaa |
| 181 | tggccaaatg gttccttgtc ctatagctgt agcagccatc ggctgttagt gacaaagccc |
| 241 | ctgagtcaag atgacagcag cccccataac tcctaatcgg ctctcccgcg tggagtcatt |
| 301 | taggagtagt cgcattagag acaagtccaa catctaatct tccaccctgg ccagggcccc |
| 361 | agctggcagc gagggtggga gactccgggc agagcagagg gcgctgacat tggggcccgg |
| 421 | cctggcttgg gtccctctgg cctttcccca ggggccctct ttccttgggg ctttcttggg |
| 481 | ccgccactgc tcccgctcct ctccccccat cccaccccct caccccctcg ttcttcatat |
| 541 | ccttctctag tgctccctcc accttcatcc acccttctgc aagagtgtgg gaccacaaat |
| 601 | gagttttcac ctggcctggg gacacacgtg cccccacagg tgctgagtga ctttctagga |
| 661 | cagtaatctg ctttaggcta aaatgggact tgatcttctg ttagccctaa tcatcaatta |
| 721 | gcagagccgg tgaaggtgca gaacctaccg cctttccagg cctcctccca cctctgccac |
| 781 | ctccactctc cttcctggga tgtgggggct ggcacacgtg tggcccaggg cattggtggg |
| 841 | attgcactga gctgggtcat tagcgtaatc ctggacaagg gcagacaggg cgagcggagg |
| 901 | gccagctccg gggctcaggc aaggctgggg gcttccccca gacaccccac tcctcctctg |
| 961 | ctggaccccc acttcatagg gcacttcgtg ttctcaaagg gcttccaaat agcatggtgg |
| 1021 | ccttggatgc ccagggaagc ctcagagttg cttatctccc tctagacaga aggggaatct |
| 1081 | cggtcaagag ggagaggtcg ccctgttcaa ggccacccag ccagctcatg gcggtaatgg |
| 1141 | gacaaggctg gccagccatc ccaccctcag aagggacccg gtggggcagg tgatctcaga |
| 1201 | ggaggctcac ttctgggtct cacattcttg gatccggttc caggcctcgg ccctaaatag |
| 1261 | tctccctggg ctttcaagag aaccacatga gaaaggagga ttcgggctct gagcagtttc |
| 1321 | accacccacc ccccagtctg caaatcctga cccgtgggtc cacctgcccc aaaggcggac |
| 1381 | gcaggacagt agaagggaac agagaacaca taaacacaga gagggccaca gcggctccca |
| 1441 | cagtcaccgc caccttcctg gcggggatgg gtggggcgtc tgagtttggt tcccagcaaa |
| 1501 | tccctctgag ccgcccttgc gggctcgcct caggagcagg ggagcaagag gtgggaggag |
| 1561 | gaggtctaag tcccaggccc aattaagaga tcaggtagtg tagggtttgg gagcttttaa |
| 1621 | ggtgaagagg cccgggctga tcccacaggc cagtataaag cgccgtgacc ctcaggtgat |
| 1681 | gcgccagggc cggctgccgt cggggacagg gctttccata gccagg, |

or a functional fragment of a variant having at least about 50%, or at least about 60%, or at least about 70%, or at least about 80%, or at least about 85%, or at least about 90%, or at least about 93%, or at least about 95%, or at least about 97%, or at least about 98% identity thereto.

In embodiments, the retina-specific promoter is the RPE promoter that comprises a nucleic acid sequence of SEQ ID NO: 3, SEQ ID NO: 4, or SEQ ID NO: 5, or a variant having at least about 80%, or at least about 85%, or at least about 90%, or at least about 93%, or at least about 95%, or at least about 97%, or at least about 98% identity thereto.

In embodiments, the retina-specific promoter is the photoreceptor promoter that comprises a nucleic acid sequence of SEQ ID NO: 6, SEQ ID NO: 7, SEQ ID NO: 8, or SEQ ID NO: 9, or a functional fragment of a variant having at least about 50%, or at least about 60%, or at least about 70%, or at least about 80%, or at least about 85%, or at least about 90%, or at least about 93%, or at least about 95%, or at least about 97%, or at least about 98% identity thereto.

In embodiments, the present non-viral vectors may comprise at least one pair of an inverted terminal repeat at the 5′ and 3′ ends of the transposon. In embodiments, an inverted terminal repeat is a sequence located at one end of a vector that can form a hairpin structure when used in combination with a complementary sequence that is located at the opposing end of the vector. The pair of inverted terminal repeats is involved in the transposition activity of the transposon of the non-viral vector of the present disclosure, in particular involved in DNA addition or removal and excision of DNA of interest. In one embodiment, at least one pair of an inverted terminal repeat appears to be the minimum sequence required for transposition activity in a plasmid. In another embodiment, the vector of the present disclosure may comprise at least two, three or four pairs of inverted terminal repeats. As would be understood by the person skilled in the art, to facilitate ease of cloning, the necessary terminal sequence may be as short as possible and thus contain as little inverted repeats as possible. Thus, in one embodiment, the vector of the present disclosure may comprise not more than one, not more than two, not more than three or not more than four pairs of inverted terminal repeats: in one embodiment, the vector of the present disclosure may comprise only one inverted terminal repeat.

In embodiments, the inverted terminal repeat of the present invention may form either a perfect inverted terminal repeat (or interchangeably referred to as “perfect inverted repeat”) or imperfect inverted terminal repeat (or interchangeably referred to as “imperfect inverted repeat”). As used herein, the term “perfect inverted repeat” refers to two identical DNA sequences placed at opposite direction. In contrast, the term “imperfect inverted repeat” refers to two DNA sequences that are similar to one another except that they contain a few mismatches. These repeats (i.e. both perfect inverted repeat and imperfect inverted repeat) are the binding sites of transposase.

In some embodiments, the ITRs of the non-viral vector are those of a piggyBac-like transposon, optionally comprising a TTAA repetitive sequence, and/or the ITRs flank the ABCA4. The piggyBac-like transposon transposes through a “cut-and-paste” mechanism, and the piggyBac-like transposon can comprise a TTAA repetitive sequence. The piggyBac transposon is a frequently used transposon system for gene modifications and does not require DNA synthesis during the actual transposition event. The piggyBac element can be cut down from the donor chromosome by a transposase, and the split donor DNA can be reconnected with DNA ligase. Zhao et al. Translational lung cancer research, 2006; 5(1):120-125. The piggyBac transposon shows precise excision, i.e., restoring the sequence to its pre-integration state. Yusa. piggyBac Transposon. Microbiol Spectr. 2015 April; 3(2). In some embodiments, the gene transfer construct comprises a Super piggyBac™ (SPB) transposase. See Barnett et al. Blood 2016; 128(242167.

In some embodiments, other non-viral gene transfer tools can be used such as, for example, the Sleeping Beauty transposon system. See, e.g., Aronovich et al. Human Molecular Genetics, 2011; 20(R1), R14—R20.

In some embodiments, sequences of the transposon systems can be codon optimized to provide improved mRNA stability and protein expression in mammalian systems.

In various embodiments, the gene transfer construct can be any suitable genetic construct, such as a nucleic acid construct, a plasmid, or a vector. In various embodiments, the gene transfer construct is DNA. In some embodiments, the gene transfer construct is RNA. In some embodiments, the gene transfer conduct can have DNA sequences and RNA sequences.

In embodiments, the present nucleic acids include polymeric form of nucleotides of any length, either ribonucleotides or deoxyribonucleotides, or analogs or derivatives thereof. In embodiments, there is provided double- and single-stranded DNA, as well as double- and single-stranded RNA, and RNA-DNA hybrids. In embodiments, transcriptionally-activated polynucleotides such as methylated or capped polynucleotides are provided. In embodiments, the present compositions are mRNA or DNA.

In embodiments, the present non-viral vectors are linear or circular DNA molecules that comprise a polynucleotide encoding a polypeptide and is operably linked to control sequences, wherein the control sequences provide for expression of the polynucleotide encoding the polypeptide. In embodiments, the non-viral vector comprises a promoter sequence, and transcriptional and translational stop signal sequences. Such vectors may include, among others, chromosomal and episomal vectors, e.g., vectors derived from bacterial plasmids, from transposons, from yeast episomes, from insertion elements, from yeast chromosomal elements, and vectors derived from combinations thereof. The present constructs may contain control regions that regulate as well as engender expression.

In some embodiments, the gene transfer construct can be codon optimized. In the described embodiments, nucleic acid encoding the ABCA4, or a functional fragment thereof, function as transgenes that are integrated into a host genome (e.g., a human genome) to provide desired clinical outcomes. Transgene codon optimization can be used to optimize therapeutic potential of the transgene and its expression in the host organism. Codon optimization is performed to match the codon usage in the transgene with the abundance of transfer RNA (tRNA) for each codon in a host organism or cell. Codon optimization methods are known in the art and described in, for example, WO 2007/142954, which is incorporated by reference herein in its entirety. Optimization strategies can include, for example, the modification of translation initiation regions, alteration of mRNA structural elements, and the use of different codon biases.

The gene transfer construct includes several other regulatory elements that are selected to ensure stable expression of the construct. Thus, in some embodiments, the non-viral vector is a DNA plasmid that can comprise one or more insulator sequences that prevent or mitigate activation or inactivation of nearby genes. In some embodiments, the one or more insulator sequences comprise an HS4 insulator (1.2-kb 5′-HS4 chicken β-globin (cHS4) insulator element) and an D4Z4 insulator (tandem macrosatellite repeats linked to Facio-Scapulo-Humeral Dystrophy (FSHD). In some embodiments, the sequences of the HS4 insulator and the D4Z4 insulator are as described in Rival-Gervier et al. Mol Ther. 2013 August; 21(8):1536-50, which is incorporated herein by reference in its entirety. In some embodiments, the gene of the gene transfer construct is capable of transposition in the presence of a transposase. In some embodiments, the non-viral vector in accordance with embodiments of the present disclosure comprises a nucleic acid construct encoding a transposase. The transposase can be an RNA transposase plasmid. In some embodiments, the non-viral vector further comprises a nucleic acid construct encoding a DNA transposase plasmid. In some embodiments, the transposase is an in vitro-transcribed mRNA transposase. The transposase is capable of excising and/or transposing the gene from the gene transfer construct to site- or locus-specific genomic regions.

A composition comprising a gene transfer construct in accordance with the present disclosure can include one or more non-viral vectors. Also, the transposase can be disposed on the same (cis) or different vector (trans) than a transposon with a transgene. Accordingly, in some embodiments, the transposase and the transposon encompassing a transgene are in cis configuration such that they are included in the same vector. In some embodiments, the transposase and the transposon encompassing a transgene are in trans configuration such that they are included in different vectors. The vector is any non-viral vector in accordance with the present disclosure.

In some embodiments, the transposase is derived from Bombyx mori, Xenopus tropicalis, Trichoplusia ni, Rhinolophus ferrumequinum, Rousettus aegyptiacus, Phyllostomus discolor; Myotis myotis, Myotis lucifugus, Pteropus vampyrus, Pipistrellus kuhlii, Pan troglodytes, Molossus molossus, or Homo sapiens, and/or is an engineered version thereof. In some embodiments, the transposase specifically recognizes the ITRs. The transposase can include DNA or RNA sequences encoding Bombyx mori, Xenopus tropicalis, or Trichoplusia ni proteins. See, e.g., U.S. Pat. No. 10,041,077, which is incorporated herein by reference in its entirety.

In some embodiments, however, a transposase may be introduced into the cell directly as protein, for example using cell-penetrating peptides (e.g., as described in Ramsey and Flynn. Pharmacol. Ther 2915; 154: 78-86); using small molecules including salt plus propanebetaine (e.g., as described in Astolfo et al. Cell 2015; 161:674-690); or electroporation (e.g., as described in Morgan and Day. Methods in Molecular Biology 1995; 48: 63-71).

In some embodiments, the transposon system can be implemented as described, e.g., in U.S. Pat. No. 10,435,696, which is incorporated herein by reference in its entirety.

In some embodiments, the described composition includes a transgene (e.g., ABCA4 or a functional fragment thereof) and a transposase in a certain ratio. In some embodiments, a transgene to transposase ratio is selected that improves efficiency of transpositional activity. The transgene to transposase ratio can be dependent on the concentration of the transfected gene transfer construct, and other factors. In some embodiments, the ratio of the nucleic acid encoding the ABCA4, or a functional fragment thereof, to the nucleic acid construct encoding the transposase is about 5:1, or about 4:1, or about 3:1, or about 2:1, or about 1:1, or about 1:2, or about 1:3, or about 1:4, or about 1:5. In some embodiments, the ratio of the nucleic acid encoding the ABCA4 protein to the nucleic acid construct encoding the transposase is about 2:1. In some aspects, a composition comprising a gene transfer construct is provided, in embodiments, the composition comprises (a) a nucleic acid encoding an ATP Binding Cassette Subfamily A Member 4 (ABC) transporter (ABCA4) protein, or a functional fragment thereof; (b) CAG promoter; and (c) a non-viral vector comprising one or more transposase recognition sites and one or more inverted terminal repeats (ITRs) or end sequences, wherein the ABCA4 protein is human ABCA4 protein, or a functional fragment thereof, that comprises a nucleotide sequence encoding a protein having an amino acid sequence of SEQ ID NO: 1, or a variant having at least about 95% identity thereto.

In some aspects, a composition comprising a gene transfer construct is provided. In embodiments, the composition comprises (a) a nucleic acid encoding an ATP Binding Cassette Subfamily A Member 4 (ABC) transporter (ABCA4) protein, or a functional fragment thereof; (b) CAG promoter; and (c) a non-viral vector comprising one or more transposase recognition sites and one or more inverted terminal repeats (ITRs) or end sequences, wherein the ABCA4 protein is human ABCA4, or a functional fragment thereof, that is encoded by a nucleotide sequence of SEG ID NO: 2, or a variant having at least about 95% identity thereto.

In some aspects, a method for treating and/or mitigating Inherited Macular Degeneration (IMD) is provided, comprising: (a) contacting a cell obtained from a patient or another individual with a composition of claim 62; (b) contacting the cell with a nucleic acid construct encoding a transposase that is derived from Bombyx mori, Xenopus tropicalis, Trichoplusia ni, Rhinolophus ferrumequinum, Rousettus aegyptiacus, Phyllostomus discolor, Myotis myotis, Myotis lucifugus, Pteropus vampyrus, Pipistrellus kuhlii, Pan troglodytes, Molossus molossus, or Homo sapiens, and/or an engineered version thereof, wherein the ratio of the nucleic acid encoding the ABCA4 protein, or a functional fragment thereof to the nucleic acid construct encoding the transposase is about 2:1; and (c) administering the cell to a patient in need thereof.

In some embodiments, the non-viral vector is a conjugated polynucleotide sequence that is introduced into cells by various transfection methods such as, e.g., methods that employ lipid particles. In some embodiments, a composition, including a gene transfer construct, comprises a delivery particle. In some embodiments, the delivery particle comprises a lipid-based particle (e.g., a lipid nanoparticle (LNP)), cationic lipid, or a biodegradable polymer). Lipid nanoparticle (LNP) delivery of gene transfer construct provides certain advantages, including transient, non-integrating expression to limit potential off-target events and immune responses, and efficient delivery with the capacity to transport large cargos. LNPs have been used for delivery of mRNA into the retina. See Patel et al., J Control Release. 2019 Jun. 10; 303:91-100. doi: 10.1016/fj.jconrel.2019.04.015. Epub 2019 Apr. 12. Also, U.S. Pat. No. 10,195,291, for example, describes the use of LNPs for delivery of RNA interference (RNAi) therapeutic agents.

In some embodiments, the composition in accordance with embodiments of the present disclosure is in the form of an LNP. In some embodiments, the LNP comprises one or more lipids selected from 1,2-dioleoyl-3-trimethylammonium propane (DOTAP); N,N-dioleyl-N,N-dimethylammonium chloride (DODAC); N-(2,3-dioleyloxy)propyl)-N,N,N-trimethylammonium chloride (DOTMA); N,N-distearyl-N,N-dimethylammonium bromide (DDAB), a cationic cholesterol derivative mixed with dimethylaminoethane-carbamoyl (DC-Chol), phosphatidylcholine (Pc, triolein (glyceryl trioleate), and 1,2-distearoyl-sn-glycero-3-phosphoethanolamine-N-[carboxy(polyethylene glycol)-2000] (DSPE-PEG), 1,2-dimyristoyl-rac-glycero-3-methoxypolyethyleneglycol-2000 (DMG-PEG 2K), and 1,2 distearol-sn-glycerol-3phosphocholine (DSPC).

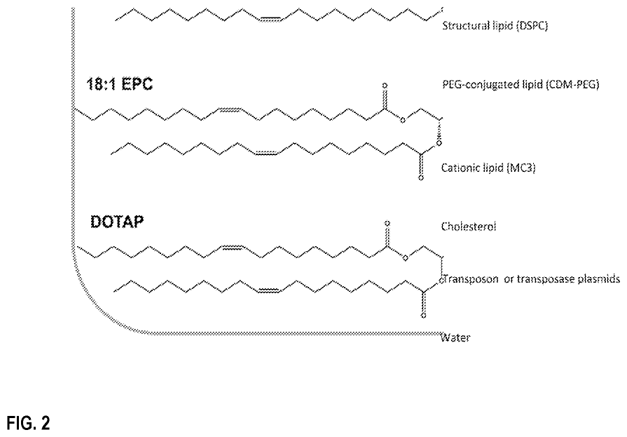

In some embodiments, an LNP can be as shown in FIG. 2, which is adapted from Patel et al., J Control Release 2019; 303:91-100. As shown in FIG. 2, the LNP can comprise one or more of a structural lipid (e.g. DSPC), a PEG-conjugated lipid (CDM-PEG), a cationic lipid (MCS), cholesterol, and a targeting ligand (e.g. GalNAc).

In some embodiments, the composition can have a lipid and a polymer in various ratios, wherein the lipid can be selected from, e.g., DOTAP, DC-Chol, PC, Triolein, DSPE-PEG, and wherein the polymer can be, e.g., PEI or Poly Lactic-co-Glycolic Acid (PLGA). Any other lipid and polymer can be used additionally or alternatively. In some embodiments, the ratio of the lipid and the polymer is about 0.5:1, or about 1:1, or about 1:1.5, or about 1:2, or about 1:2.5, or about 1:3, or about 3:1, or about 2.5:1, or about 2:1, or about 1.5:1, or about 1:1, or about 1:0.5.

In some embodiments, the LNP comprises a cationic lipid, non-limiting examples of which include N,N-dioleyl-N,N-dimethylammonium chloride (DODAC), N,N-distearyl-N,N-dimethylammonium bromide (DDAB), N-(l-(2,3-dioleoyloxy)propyl)-N,N,N-trimethylammonium chloride (DOTAP), N-(l-(2,3-dioleyloxy)propyl)-N,N,N-trimethylammonium chloride (DOTMA), N,N-dimethyl-2,3-dioleyloxy)propylamine (DODMA), 1,2-DiLinoleyloxy-N,N-dimethylaminopropane (DLinDMA), 1,2-Dilinolenyloxy-N,N-dimethylaminopropane (DLenDMA), 1,2-Dilinoleylcarbamoyloxy-3-dimethylaminopropane (DLin-C-DAP), 1,2-Dilinoleyoxy-3-(dimethylamino)acetoxypropane (DLin-DAC), 1,2-Dilinoleyoxy-3-morpholinopropane (DLin-MA), 1,2-Dilinoleoyl-3-dimethylaminopropane (DLinDAP), 1,2-Dilinoleyithio-3-dimethylaminopropane (DLin-S-DMA), 1-Linoleoyl-2-linoleyloxy-3-dimethylaminopropane (DLin-2-DMAP), 1,2-Dilinoleyloxy-3-trimethylaminopropane chloride salt (DLin-TMA.Cl), 1,2-Dilinoleoyl-3-trimethylaminopropane chloride salt (DLin-TAP.Cl), 1,2-Dilinoleyloxy-3-(N-methylpiperazino)propane (DLin-MPZ), or 3-(N,N-Dilinoleylamino)-1,2-propanediol (DLinAP), 3-(N,N-Dioleylamino)-1,2-propanedio (DOAP), 1,2-Dilinoleyloxo-3-(2-N,N-dimethylamino)ethoxypropane (DLin-EG-DMA), 1,2-Dilinolenyloxy-N,N-dimethylaminopropane (DLinDMA), 2,2-Dilinoleyl-4-dimethylaminomethyl-[1,3]-dioxolane (DLin-K-DMA) or analogs thereof, (3aR,5s,6aS)-N,N-dimethyl-2,2-di((9Z,12Z)-octadeca-9,12-dienyl)tetrahydro-3aH-cyclopenta[d][1,3]dioxol-5-amine (ALN100), (6Z,9Z,28Z,31Z)-heptatriaconta-6,9,28,31-tetraen-19-yl 4-(dimethylamino)butanoate (MC3), 1,1′-(2-(4-(2-((2-(bis(2-′)amino)ethyl)(2 hydroxydodecyl)amino)ethyl)piperazin-1-yl)ethylazanediyl)didodecan-2-ol (Tech G1), or a mixture thereof.

In some embodiments, the LNP comprises one or more molecules selected from polyethylenimine (PEI) and poly(lactic-co-glycolic acid) (PLGA), and N-Acetylgalactosamine (GalNAc), which are suitable for hepatic delivery, In some embodiments, the LNP comprises a hepatic-directed compound as described, e.g., in U.S. Pat. No. 5,985,826, which is incorporated by reference herein in its entirety. GalNAc is known to target Asialoglycoprotein Receptor (ASGPR) expressed on mammalian hepatic cells, See Hu et al. Protein Pept Lett 2014; 21(10):1025-30.

In some examples, the gene transfer constructs of the present disclosure can be formulated or complexed with PEI or a derivative thereof, such as polyethyleneimine-polyethyleneglycol-N-acetylgalactosamine (PEI-PEG-GAL) or polyethyleneimine-polyethyleneglycol-tri-N-acetylgalactosamine (PEI-PEG-triGAL) derivatives.

In some embodiments, the LNP is a conjugated lipid, non-limiting examples of which include a polyethyleneglycol (PEG)-lipid including, without limitation, a PEG-diacylglycerol (DAG), a PEG-dialkyloxypropyl (DAA), a PEG-phospholipid, a PEG-ceramide (Cer), or a mixture thereof. The PEG-DAA conjugate may be, for example, a PEG-dilauryloxypropyl (C12, a PEG-dimyristyloxypropyl (C14), a PEG-dipalmityloxypropyl (C16), or a PEG-distearyloxypropyl (C18).

In embodiments, a nanoparticle is a particle having a diameter of less than about 1000 nm. In some embodiments, nanoparticles of the present disclosure have a greatest dimension (e.g. diameter) of about 500 nm or less, or about 400 nm or less, or about 300 nm or less, or about 200 nm or less, or about 100 nm or less. In some embodiments, nanoparticles of the present invention have a greatest dimension ranging between about 50 nm and about 150 nm, or between about 70 nm and about 130 nm, or between about 80 nm and about 120 nm, or between about 90 nm and about 110 nm, in some embodiments, the nanoparticles of the present invention have a greatest dimension (e.g., a diameter) of about 100 nm.

In some aspects, the compositions in accordance with the present disclosure can be delivered via an in vivo genetic modification method. In some embodiments, a genetic modification in accordance with the present disclosure can be performed via an ex vivo method.

Accordingly, in some embodiments, a method for preventing or decreasing the rate of photoreceptor loss in a patient is provided that comprises administering to a patient in need thereof a composition according to any embodiment, or a combination of embodiments, of the present disclosure. The method includes delivering the composition via a suitable route, including administering by injection.

In some embodiments, the present methods and compositions can provide durable prevention or decreasing of the rate of photoreceptor loss, and the need for additional therapeutic agents can therefore be decreased or eliminated. For example, in some embodiments, the method is performed in the absence of a steroid treatment. The method can be substantially non-immunogenic.

In some aspects, the present invention provides an ex vivo gene therapy approach. Accordingly, in some aspects, a method for preventing or decreasing the rate of photoreceptor loss in a patient is provided that comprises (a) contacting a cell obtained from a patient (autologous) or another individual (allogeneic) with a composition in accordance with embodiments of the present disclosure; and (b) administering the cell to a patient in need thereof.

In some aspects, the method for treating and/or mitigating an inherited Macular Degeneration (IMD) is provided that comprises administering to a patient in need thereof a composition in accordance with embodiments of the present disclosure, in such in vivo method, the composition is administered using any of the techniques described herein.

In some embodiments, the in vivo and ex viva methods described herein can treat and slow progression of various MDs which are a heterogeneous group of disorders characterized by bilateral symmetrical central visual loss. MDs include Stargardt disease, Best disease, X-linked retinoschisis, pattern dystrophy, Sorsby fundus dystrophy, and autosomal dominant drusen. Best disease is an autosomal dominant condition associated with disease-causing variants in BEST1; X-linked retinoschisis (XLRS) is the most common form of juvenile-onset retinal degeneration in male adolescents; pattern dystrophy (PD) is a group of disorders characterized by variable distributions of pigment deposition at the level of the RPE; Sorsby fundus dystrophy (SFD) is a rare macular dystrophy often leading to bilateral central visual loss in the fifth decade of life; and autosomal dominant drusen (ADD) is an autosomal dominant condition characterized by drusen-like deposits at the macula, which may have a radiating or honeycomb-like appearance. See Rahman et al., Br Ophthalmol. 2020; 104(4):451-460.

In some aspects, an ex vivo method for treating and/or mitigating an IMD is provided that comprises (a) contacting a cell obtained from a patient or another individual with a composition in accordance with embodiments of the present disclosure, and (b) administering cells to a patient in need thereof: in some embodiments, the IMD is a STGD. In some embodiments, the STGD is STGD Type 1 (STGD1). In some embodiments, the STGD disease can be STGD Type 3 (STGD3) or STGD Type 4 (STGD4) disease.

In some embodiments, the IMD is characterized by one or more mutations in one or more of ABCA4, ELOVL4, PROM1, BEST1, and PRPH2. In some embodiments, the ABCA4 mutations are autosomal recessive mutations.

Mutations in ELOVL4 (elongation of very long chain fatty acids protein 4) were shown to cause STGD3 characterized by retinal degeneration. Agbaga et al., PNAS Sep. 2, 2008; 105 (35) 12843-12848; see also Zhang et al., Nat Genet. 2001; January; 27(1):89-93. The clinical profile of STGD3 is very similar to STGD1.

PROM1 (prominin 1 gene) encodes a pentaspan transmembrane glycoprotein, which is a protein localized to membrane protrusions. Yang et al., J Clin invest. 2008; 118(8):2908-2916. Mutations in PROM1 gene have been shown to result in retinitis pigmentosa and Stargardt disease, and this gene is expressed from at least five alternative promoters that are expressed in a tissue-dependent manner. See, e.g., Lönnroth et al., Int J Oncol. 2014; 45(6):2208-2220.

The BEST1 gene provides instructions for making a protein called bestrophin-1, which appears to play a critical role in normal vision. Mutations in the BESTI gene cause detachment of the retina and degeneration of photoreceptor (PR) cells due to a primary channelopathy in the neighboring RPE cells. Guziewicz et al., PNAS Mar. 20, 2018 115 (12) E2839-E2848; see also Petrukhin et al., Nature Genetics 1998; vol.19:241-247. Disease-causing variants in BEST1 have been linked to Best Disease (BD), which is the second most common MD, affecting approximately 1 in 10 000. Rahman et al., Br J Ophthalmol. 2020 April; 104(4):451-460. BEST1 sequence variants also account for at least four other phenotypes, such as adult vitelliform MD, autosomal dominant vitreochoroidopathy, autosomal recessive bestrophinopathy, and retinitis pigmentosa. Id.

The PRPH2 (peripherin-2) gene encodes a PR-specific tetraspanin protein called peripherin-2/retinal degeneration slow (RDS), arid mutations in PRPH2 have been shown to cause forms of retinitis pigmentosa and macular degeneration. Conley & Naash. Cold Spring Harb Perspect Med. 2014 Aug. 28; 4(11):a017376: Mutations in PRPH2 have been identified in patients with Stargardt macular degeneration.

The pathogenic mutations in one or more of ABCA4, ELOVL4, PROM1, BEST1 and PRPH2 can be corrected using the described methods for treating and/or mitigating related macular dystrophy conditions.

One of the advantages of ex vivo gene therapy is the ability to “sample” the transduced cells before patient administration: This facilitates efficacy arid allows performing safety checks before introducing the cell(s) to the patient, For example, the transduction efficiency and/or the clonality of integration can be assessed before infusion of the product. The present disclosure provides compositions and methods that can be effectively used for ex vivo gene modification.

In some embodiments, any of the in vivo and ex vivo methods described herein improve distance visual acuity of the patient of the patient, In some embodiments, the method is substantially non-immunogenic.

In some embodiments, the method requires a single administration, which simplifies the delivery of the present composition and improves overall patient experience. Many patients afflicted by various IMDs disorders are children, and delivering a durable, substantially non-immunogenic treatment in accordance with some embodiments of the present disclosure—as a one-time administration—facilitates the therapy delivery process and decreases the burden on the patient.

As mentioned above, accumulation of lipofuscin in the RPE has been associated with the development of STGD, age-related macular degeneration, and other retinal diseases. The dumps of lipofuscin, a yellow substance that forms flecks, accumulate in and around the macula, impairing central vision. A main component of lipofuscin is the bis-retinoid N-retinylidene-N-retinylethanolamine (A2E), though lipofuscin includes other bis-retinoids. A2E is a fluorescent material that accumulates, with age or in some retinal disorders such as STGD, in the lysosomes of RPE of the eye. RPE lipofuscin includes A2E and an additional fluorophore—a double bond isomer of A2E, iso-A2E. Studies on the photochemistry of A2E and iso-A2E indicated that they exist in a photoequilibrium of 4 4 (A2E):1 (iso-A2E). See Parish et al., Proc Natl Acad Sci USA. 1998; 95(25):14609-13. A2E was shown to trigger the accumulation of lipofuscin-like debris in the RPE. Mihai & Washington. Cell Death & Disease 5, e1348(2014). A2E can be responsible for RPE debris found in the human eye, which encompass lipofuscin-like bodes, late-stage lysosomes, abnormal glycogen and lipid deposits, and inclusions that show heterogeneous electron density. Id. A2E thus drives retinal senescence and associated degeneration. A2E's chemical precursor, vitamin A aldehyde (retinaldehyde), also plays a role in the degenerative process. Id.

Accordingly, lowering levels of one or more of retinaldehyde, A2E, and iso-A2E can treat or mitigate lipofuscin accumulation in the retina, e.g., in the RPE and/or the underlying Bruch's membrane, in some embodiments, the method reduces or prevents the formation of RPE debris. In some embodiments, the lowering levels of one or more of retinaldehyde, A2E, and iso-A2E can treat or mitigate accumulation of vitamin A dimers in the RPE and Bruch's membrane (BM).

Accordingly, in some embodiments, the method provides a lowering of one or more of retinaldehyde, N-retinylidene-N-retinylethanolamine (A2E) and iso-A2E relative to a level of one or more of retinaldehyde, A2E, and iso-A2E without the administration of the present composition. In some embodiments, levels of one or more of retinaldehyde, A2E, and iso-A2E are lowered (relative to a level of one or more of retinaldehyde, A2E, and iso-A2E without the administration of the present composition) are lowered by greater than at least about a 40%. In some embodiments, the method provides greater than about a 40%, or greater than about a 50%, or greater than about a 60%, or greater than about a 70%, or greater than about a 80%, or greater than about a 90% lowering.

In some embodiments, a nucleic acid construct encoding a transposase is administering to the patient. The transposase can be derived from Bombyx mori, Xenopus tropicalis, Trichoplusia ni, Rhinolophus ferrumequinum, Rousettus aegyptiacus, Phyllostomus discolor, Myotis myotis, Myotis lucifugus, Pteropus vampyrus, Pipistrellus kuhlii, Pan troglodytes, Molossus molossus, or Homo sapiens, and/or an engineered version thereof.

In some embodiments, the ex vivo method for preventing or decreasing the rate of photoreceptor loss in a patient comprises contacting the cells with a nucleic acid construct encoding a transposase, optionally derived from Bombyx mori, Xenopus tropicalis, Trichoplusia ni, Rhinolophus ferrumequinum, Rousettus aegyptiacus, Phyllostomus discolor, Myotis myotis, Myotis lucifugus, Pteropus vampyrus, Pipistrellus kuhlii, Pan troglodytes, Molossus molossus, or Homo sapiens, and/or an engineered version thereof.

In some embodiments, the method for preventing or decreasing the rate of photoreceptor loss in a patient is performed in the absence of a steroid treatment. Steroids, such as glucocorticoid steroids (e.g., prednisone) have been used to improve effectiveness of AAV-based gene therapy by reducing immune response. However, steroid treatment is not without side effects. The compositions and methods of the present disclosure can be substantially non-immunogenic, arid can therefore eliminate the need for a steroid treatment.

In some embodiments, however, the methods are performed in combination with a steroid treatment.

In some embodiments, the method can be used to administer the described composition in combination with one or more additional therapeutic agents. Non-limiting examples of the additional therapeutic agents comprise one or more of an anti-Vascular endothelial growth factor (VEGF) therapeutic agents including aflibercept (EYLEA), ranibizumab (LUCENTIS), and bevacizumab (Avastin). The additional therapeutic agents can include deuterated vitamin A and/or other vitamins or nutritional supplements (e.g., beta carotene, lutein, and zeaxanthin).

The administration can be intra-vitreal or intra-retinal. In some embodiments, the administering is to RPE cells and/or photoreceptors. The compositions for non-viral gene therapy in accordance with the present disclosure can be administered via various delivery routes, including the administration by injection. In some embodiments, the injection is intra-vitreal or intra-retinal. In some embodiments, the injection is sub-vitreal or sub-retinal. In some embodiments, the injection is sub-RPE.

In some embodiments, the in vitro or ex vivo method for treating and/or mitigating an IMD provides improved distance visual acuity and/or decreased the rate of photoreceptor loss as compared to a lack of treatment. In some embodiments, the method results in improvement of best corrected visual acuity (BOVA) to greater than about 20/200.

In some embodiments, the method for treating and/or mitigating an IMD results in improvement of retinal or foveal morphology, as measured by fundus autofluorescence (FAF) or Spectral Domain-Optical Coherence Tomography (SD-OCT). FAF is a non-invasive retinal imaging modality used to provide a density map of lipofuscin in the retinal pigment epithelium. See Madeline et al., Int J Retin Vitr 2, 12 (2016); Sepah et al:, Saudi J Ophthalmol. 2014; 28(2):111-116; Sparrow et al., Investigative Ophthalmology & Visual Science September 2010; vol,51:4351-4357.

SD-OCT is an interferometric technique that provides depth-resolved tissue structure information encoded in the magnitude and delay of the back-scattered light by spectral analysis of the interference fringe pattern. Yaqoob et al., Biotechniques, vol. 39, No. 6S; published Online:30 May 2018. Other imaging technologies can be used as well, including, e.g., a scanning laser ophthalmoscopy (SLO), Fluorescence lifetime imaging ophthalmoscopy (FLIO), and two-photon microscopic imaging (TPM). Images (of one or both eyes) acquired using a suitable technology can be analyzed to assess parameters of a patient, including fluorescence intensity. For example, FAF that is characterized by a general increase of autofluorescence intensity is indicative of the Stargardt disease, at early stages of the disease. Burke et al., Invest Ophthalmol Vis Sci. 2014; 55: 2841 —2852.

In some embodiments, the method results in reduction or prevention of one or more of wavy vision, blind spots, blurriness, loss of depth perception, sensitivity to glare, impaired color vision, and difficulty adapting to dim lighting (delayed dark adaptation) in the patient.

In some embodiments, the method can be used to administer the described composition in combination with one or more additional therapeutic agents. Non-limiting examples of the additional therapeutic agents comprise one or more of Soraprazan, Isotretinoin, Dobesilate, 4-methylpyrazole, ALK-001 9 (C20 deuterated vitamin A), Fenretinide (a synthetic form of vitamin A), LBS-500, A1120, Emixustat, Fenofibrate, and Avacincaptad pegol. In some embodiments, the method obviates the need for an additional therapeutic agent, which can be any of the above therapeutic agents.

In some embodiments, the method obviates the need for steroid treatment.

In some embodiments, the composition in accordance with the present disclosure comprises a pharmaceutically acceptable carrier, excipient or diluent.

Methods of formulating suitable pharmaceutical compositions are known in the art, see, e.g., Remington: The Science and Practice of Pharmacy, 21st ed., 2005; and the books in the series Drugs and the Pharmaceutical Sciences: a Series of Textbooks and Monographs (Dekker, N.Y.). For example, pharmaceutical compositions suitable for injectable use can include sterile aqueous solutions (where water soluble) or dispersions and sterile powders for the extemporaneous preparation of sterile injectable solutions or dispersion. For intravenous administration, suitable carriers include physiological saline, bacteriostatic water, Gremophor EL™ (BASF, Parsippany, N.J.) or phosphate buffered saline (PBS). In all cases, the composition must be sterile and the fluid should be easy to draw up by a syringe. It should be stable under the conditions of manufacture and storage and must be preserved against the contaminating action of microorganisms such as bacteria arid fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent that delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization, Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying, which yield a powder of the active ingredient plus any additional desired ingredient from a previously sterile-filtered solution thereof.

Therapeutic compounds can be prepared with carriers that will protect the therapeutic compounds against rapid elimination from the body, such as a controlled release formulation, including implants and microencapsulated delivery systems. Biodegradable, biocompatible polymers can be used, such as collagen, ethylene vinyl acetate, polyanhydrides (e.g., poly[1,3-bis(carboxyphenoxy)propane-co-sebacic-acid] (PCPP-SA) matrix, fatty acid dimer-sebacic acid (FAD-SA) copolymer, poly(lactide-co-glycolide)), polyglycolic acid, collagen, polyorthoesters, polyethyleneglycol-coated liposomes, and polylactic acid. Such formulations can be prepared using standard techniques, or obtained commercially, e.g., from Alza Corporation and Nova Pharmaceuticals, Inc. Liposomal suspensions can also be used as pharmaceutically acceptable carriers. These can be prepared according to methods known to those skilled in the art, for example, as described in U.S. Pat. No: 4,522,811. Semisolid, gelling, soft-gel, or other formulations (including controlled release) can be used, e.g., when administration to a surgical site is desired. Methods of making such formulations are known in the art and can include the use of biodegradable, biocompatible polymers. See, e.g., Sawyer et al., Yale J Biol Med. 2006; 79(3-4): 141-152.

In embodiments, there is provided a method of transforming a cell using the gene transfer constructs described herein in the presence of a transposase to produce a stably transfected cell which results from the stable integration of a gene of interest into the cell. In embodiments, the stable integration comprises an introduction of a polynucleotide into a chromosome or mini-chromosome of the cell and, therefore, becomes a relatively permanent part of the cellular genome.

In embodiments, the present invention relates to determining whether a gene of interest, e.g. ABCA4 transferred into a genome of a host. In one embodiment, the method may include performing a polymerase chain reaction with primers flanking the gene of interest; determining the size of the amplified polymerase chain reaction products obtained; and comparing the size of products obtained with a reference size, wherein if the size of the products obtained matches the expected size, then the gene of interest was successfully transferred.

In embodiments, there is provided a host cell comprising a composition as described herein (e.g., without limitation, a composition comprising the gene transfer construct and/or transposase). In embodiments, the host cell is a prokaryotic or eukaryotic cell, e.g. a mammalian cell.

In embodiments, there is provided a transgenic organism that may comprise cells which have been transformed by the methods of the present disclosure. In one example, the organism may be a mammal or an insect. When the organism is a mammal, the organism may include, but is not limited to, a mouse, a rat, a monkey, a dog, a rabbit and the like. When the organism is an insect, the organism may include, but is not limited to, a fruit fly, a mosquito, a bollworm and the like.

The compositions can be included in a container, kit, pack, or dispenser together with instructions for administration.

Also provided herein are kits comprising: i) any of the aforementioned gene transfer constructs of this invention, and/or any of the aforementioned cells of this invention and ii) a container. In certain embodiments, the kits further comprise instructions for the use thereof. In certain embodiments, any of the aforementioned kits can further comprise a recombinant DNA construct comprising a nucleic acid sequence that encodes a transposase,

This invention is further illustrated by the following non-limiting examples.

Definitions

As used herein, “a,” “an,” or “the” can mean one or more than one.

Further, the term “about” when used in connection with a referenced numeric indication means the referenced numeric indication plus or minus up to 10% of that referenced numeric indication. For example, the language “about 50” covers the range of 45 to 55.

An “effective amount,” when used in connection with medical uses is an amount that is effective for providing a measurable treatment, prevention, or reduction in the rate of pathogenesis of a disease of interest.

As referred to herein, all compositional percentages are by weight of the total composition, unless otherwise specified. As used herein, the word “include,” and its variants, is intended to be non-limiting, such that recitation of items in a list is not to the exclusion of other like items that may also be useful in the compositions and methods of this technology. Similarly, the terms “can” and “may” and their variants are intended to be non-limiting, such that recitation that an embodiment can or may comprise certain elements or features does not exclude other embodiments of the present technology that do not contain those elements or features.

Although the open-ended term “comprising,” as a synonym of terms such as including, containing, or having, is used herein to describe and claim the invention, the present invention, or embodiments thereof, may alternatively be described using alternative terms such as “consisting of” or “consisting essentially of.”

As used herein, the words “preferred” and “preferably” refer to embodiments of the technology that afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the technology.

The amount of compositions described herein needed for achieving a therapeutic effect may be determined empirically in accordance with conventional procedures for the particular purpose. Generally, for administering therapeutic agents for therapeutic purposes, the therapeutic agents are given at a pharmacologically effective dose. A “pharmacologically effective amount,” “pharmacologically effective dose,” “therapeutically effective amount,” or “effective amount” refers to an amount sufficient to produce the desired physiological effect or amount capable of achieving the desired result, particularly for treating the disorder or disease. An effective amount as used herein would include an amount sufficient to, for example, delay the development of a symptom of the disorder or disease, after the course of a symptom of the disorder or disease (e.g., slow the progression of a symptom of the disease), reduce or eliminate one or more symptoms or manifestations of the disorder or disease, and reverse a symptom of a disorder or disease. Therapeutic benefit also includes halting or slowing the progression of the underlying disease or disorder, regardless of whether improvement is realized.

Effective amounts, toxicity, and therapeutic efficacy can be determined by standard pharmaceutical procedures in cell cultures or experimental animals, e.g., for determining the LD₅₀ (the dose lethal to about 50% of the population) and the ED₅₀ (the dose therapeutically effective in about 50% of the population). The dosage can vary depending upon the dosage form employed and the route of administration utilized. The dose ratio between toxic and therapeutic effects is the therapeutic index and can be expressed as the ratio LD₅₀/ED₅₀. In some embodiments, compositions and methods that exhibit large therapeutic indices are preferred. A therapeutically effective dose can be estimated initially from in vitro assays, including, for example, cell culture assays. Also, a dose can be formulated in animal models to achieve a circulating plasma concentration range that includes the IC₅₀ as determined in cell culture, or in an appropriate animal model. Levels of the described compositions in plasma can be measured, for example, by high performance liquid chromatography. The effects of any particular dosage can be monitored by a suitable bioassay. The dosage can be determined by a physician and adjusted, as necessary, to suit observed effects of the treatment.

As used herein, “methods of treatment” are equally applicable to use of a composition for treating the diseases or disorders described herein and/or compositions for use and/or uses in the manufacture of a medicaments for treating the diseases or disorders described herein.

### EXAMPLES

Hereinafter, the present invention will be described in further detail with reference to examples. It will be obvious to a person having ordinary skit in the art that these examples are illustrative purposes only and are not to be construed to limit the scope of the present invention. In addition, it will be apparent to those skilled in that art that various modifications and variations can be made without departing from the technical scope of the present invention.

### Example 1

### Design of Transposon Expression Vectors

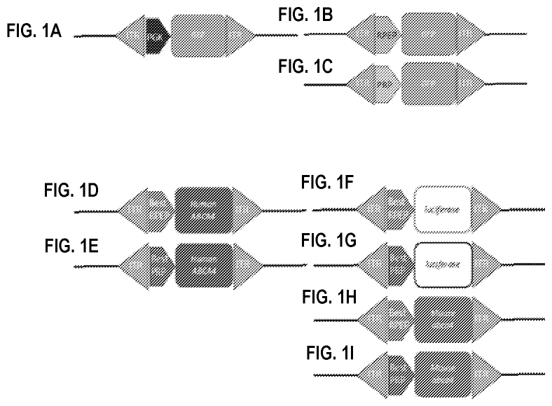

Non-viral, transposon expression vectors schematically shown in FIGS. 1A-1I are designed and cloned for in vitro, in vivo, and ex vivo studies of transfection, transposition efficacy, and expression studies in retinal cell lines.

FIG. 1A shows a phosphoglycerate kinase (PGK)-GFP transposon construct with a PGK promoter, which is used to determine a transposon (Tn): transposase (Ts) ratio and transposition efficacy by GFP fluorescent-activated cell sorting (FACS). FIGS. 1B and 1C show transposon constructs that are used to assess effectiveness if a retinal pigment epithelium promoter (RPEP) (FIG. 1B) and a photoreceptor promoter (PRP) (FIG. 1C) to selectively maximize GFP expression (determined by FACS) and copy number [determined using Droplet Digital PCR (ddPCR) or quantitative PCR (qPCR) technology].

FIG. 1D shows a BEST-RPEP construct that can be used to assess the expression of ABCA4 by flow cytometry and ABCA4 copy number (using, e.g. ddPCR or qPCR). FIG. 1E shows a BEST-PRP construct that can similarly be used to assess the expression of ABCA4 by flow cytometry and ABCA4 copy number (using, e.g. ddPCR or qPCR).

The transposon constructs shown in FIGS. 1F, 1G, 1H, and 1I are used in human iPSCs and transgenic abca4 −/− mice studies which are discussed below. The constructs in FIGS. 1F and 1H include a BEST-RPEP promoter, and constructs in FIGS. 1G and 1I include a BEST-PRP promoter.

### Example 2

### Determining the Effects of Different Transposon (Tn): Transposase (Ts) Ratios

The effects of different transposon (Tn):transposase (Ts) ratios are assessed on stable Green Fluorescent Protein (GFP) expression (>14 days) in cell lines of retinal and non-retinal origin. The study involves establishing cultures of human retinal derived adherent cell lines (ARPE-19, RPE-1) and a derived mouse photoreceptor cell line (661W). Cultures of HEK293 (ABCA4 negative) and HeLa (ABCA4 positive) cells are used as controls: in this example, the transposon vector as shown in FIG. 1A can be used. LEAPIN transposase technology can be used (ATUM, Newark, Calif.).

Different conditions for electroporation of the established cell lines can be studied, using a transposon vector expressing a GFP driven by a constitutive promoter, e.g. the vector designed as shown in FIG. 1A. Cells can be transfected with gene transfer constructs having two, three, or greater than three different Tn:Ts ratios. Conditions which result in cultures with relatively high numbers of GFP positive cells can be kept in culture by passage for 14 days. In these studies, 14 days is expected to be a sufficient period of time to allow for loss of transient expression of GFP. Transfected cultures are analyzed after 14 days by flow cytometry to determine the percentage of cells which have retained GFP expression, as a measure of stable expression. Cultures with greater than 40% GFP expression can be analyzed by ddPCR or qPCR, to determine a copy number.

### Example 3

### Selecting RPE-Specific and Photoreceptor Promoters

In this study, promoters are assessed and selected based on their ability to cause specific and high levels of GFP expression in retinal cell lines derived from the retinal pigment epithelium (RPE) or photoreceptors, in this example, the transposon vectors as shown in FIGS. 1B and 1C can be used. RPE (VMD2, IRBP, RPE65), photoreceptor [PDE, Rhodopsin kinase (Rk or GRK1), CAR (cone arrestin), RP1, L-opsin], and non-specific promoters (PGK, CAG, CMV) are cloned into transposon vectors, driving expression of GFP. The generated constructs are transfected using a certain condition (which can be identified as described in Example 2), into two human RPE cell lines (ARPE-19, RPE-1), a derived mouse photoreceptor cell line (661W), and two control cell lines (HEK293, HeLa). Relative expression levels are determined qualitatively (visually by eye or by flow cytometry), and promoters which express strongly in RPE or the photoreceptor cell line and relatively lower in the control cells, are to be considered retina-specific for purposes of this assay.

Also, in this study, ARPE-19, RPE-1, and 661W transfections with promoters considered to be RPE- and photoreceptor-specific are cultured by passage for ˜14 days and are analyzed by flow cytometry after this period. Differential levels of GFP expression are taken as a measure of the relative strengths of these promoters in the studied cell lines.

### Example 4

### Demonstrating Stable Expression of Human ABCA4 Driven by Retina-Specific Promoters in Cell Lines of Retinal and Non-Retinal Origin

Endogenous ABCA4 positive and negative controls are confirmed using HEK293 cells. HEK293 cells are used because it has been shown that ABCA4 has a similar transport function in transfected HEK293 cells as it does within the photoreceptor (see Sabirzhanova et al., J Biol Chem 2015; 290:19743-55; Quazi et al., Nat Common 2012; 3:925) and RT-PCR does not show endogenous ABCA4 expression in untransfected HEK293 (protein atlas). See Bauwens et al., Genet Med 2019; 21:1761-71. In addition, HeLa cells express endogenous ABCA4 (protein atlas). To confirm that HEK293 cells can be used as a negative control and HeLa cells can be used as a positive control, cells are labeled with an antibody against human ABCA4 using standard methods. The labeled cells are quantified by flow cytometry and visualized by immunocytochemistry techniques. Additionally, mRNA levels of endogenous ABCA4 are quantified by ddPCR or RT qPCR.

In this study, an RPE-specific promoter arid a photoreceptor promoter can be used that are selected as described in Example 3. The selected promoters are cloned into transposon vectors such as, e.g. the transposon vectors as shown in FIGS. 1D and 1E, driving expression of both human and mouse ABCA4. The transposon constructs are transfected using a transfection condition determined, e.g., as described in Example 2, into human retinal derived adherent cell lines (ARPE-19, RPE-1), and a photoreceptor cell line (661W). HEK293 (ABCA4 negative) and HeLa (ABCA4 positive) cells are used as untransfected controls. The cells are cultured by passage for ˜14 days. After this period, cultured cells were labeled using an anti-ABCA4 antibody, and the percentage of cells which express ABCA4 was quantified by flow cytometry. Percentage of fluorescent cells, analyzed by flow cytometry, is used to monitor transfection efficiency.

Additionally, the presence of ABCA4 transcript is quantified by ddPCR or RT qPCR using known methods.

### Example 5

### Generating Transposon (Tn) and Transposase (Ts) Constructs for Studies in STGD Patient iPSCs, Transgenic abca4 Mice, and Large Animal Models

The aim of this study is to identify lead transposon (Tn) and transposase (Ts) constructs for in vivo, in vitro, and ex vivo testing in patient's individual pluripotent stem cells (iPSCs), transgenic abca4 −/− mice, and large animal models (e.g. abcd4 mutant Labrador retriever). Vector constructs as shown in FIGS. 1F, 1G, 1H, and 1I can be used. The constructs can include a Luciferase (pLuc) or a GFP gene, and photoreceptor and RPE-specific promoters.

In this study, in vivo studies in Abca4 −/− transgenic mice or other animals are performed using intra-retinal delivery of transduced cell to show transposition efficacy. Thus, intra-retinal injections of a construct (using the murine Abc4a gene) into the Abca4a −/− mouse are performed to show the correction of the phenotype. Similar experiments in the naturally occurring Abca4 −/− Labrador retriever dogs (see Makelainen et at, PLoS Genet 2019; 15:e1007873) are designed to show safety, tolerability and efficacy of the appropriate constructs and administration procedure. Biodistribution, dose-response, pharmacokinetic, pharmacodynamic, safety, and pathological studies are performed in Abca4 −/− Labrador retriever dogs (or other canine models) or non-human primates (cynomolgus monkeys; Macaca fascicularis) in a GLP environment, to reverse retinal pathology.

### Example 6

Use of the MLT Transposase to Transpose 661W Mouse Photoreceptor Cells

An objective of this study was to determine the lipofection conditions to transpose 661W photoreceptor cells using the MLT transposase (RNA helper) of the present disclosure, using green fluorescent protein (GFP) driven by a CAG-GFP donor construct.

661W cells were transfected with a ratio of donor transposon DNA (CAG-GFP): MLT transposase 1 arid MLT transposase 2 mRNA (donor DNA:helper RNA) of 10 ug:5 ug. Conditions which result in cultures with relatively high numbers of GFP positive cells were kept in culture by passage for 7 to 14 days. 14 days is expected to be a sufficient period of time to allow for loss of transient expression of GFP. Cells were imaged at different time points post-transfection to monitor expression and determine which condition allowed for GFP expression out to 14 days. Optimal transfected cultures are imaged and analyzed by flow cytometry to determine the percentage of cells which have retained GFP expression. Cultures with greater than 40% GFP expression are analyzed by qPCR to determine copy number.

The following agents were used in the present study: a donor DNA (>1 ug/ul, 300 ul, 1×TE buffer, endotoxin-free, sterile), helper RNA MLT transposase 1 (>500 ng/ul, 100 ul, nuclease-free water, sterile), and helper RNA MLT transposase 2 (>500 ng/ul, 100 ul, nuclease-free water, sterile). Table 1 shows reagents used in the present study.

TABLE 1
Reagents used in the present study.

| Reagents | Supplier & Catalog Number |
|---|---|
| DNA | CAG-GFP (VB200819-1024gzm) |
| 661W Cells | |
| RNA | MLT transposase 1 (MLT1) (VB200905-1046fxw) |
| | (encodes SEQ ID NO: 13) |
| RNA | MLT transposase 2 (MLT 2) (VB200905-1047pvx) |
| | (encodes SEQ ID NO: 15) |
| Lipofectamine | ThermoFisher (Invitrogen ™) Catalog |
| 3000 (L3) | Number L3000-001 |
| Lipofectamine | ThermoFisher (Invitrogen ™) Catalog |
| LTX & PLUS | Number A12621 |
| reagent (LTX) | |
| Lipofectamine | ThermoFisher (Invitrogen ™) Catalog |
| Messenger MAX | Number LMRNA001 |
| (MAX) | |

Results

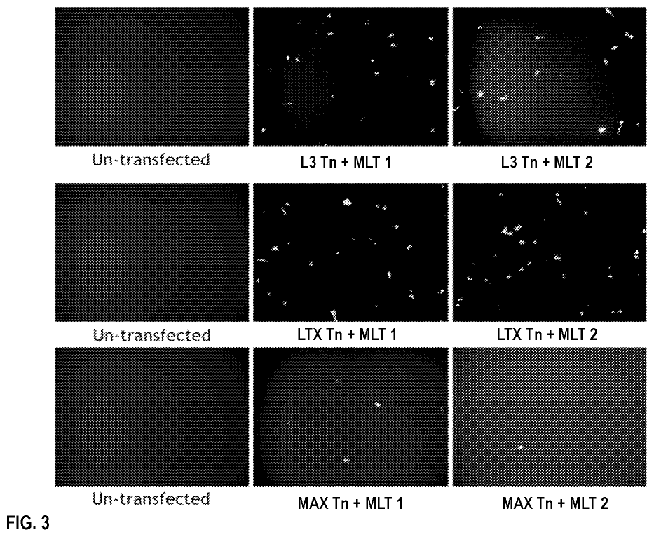

FIG. 3 shows GFP expression of 661W mouse photoreceptor cells 24 hours post transfection with varying lipofection reagents as well as either MLT transposase 1 or MLT 1 (which comprises the amino acid sequence of SEQ ID NO: 13), or MLT transposase 2 or MLT 2 (which comprises the amino acid sequence of SEQ ID NO: 15) of the present disclosure, compared to un-transfected cells.

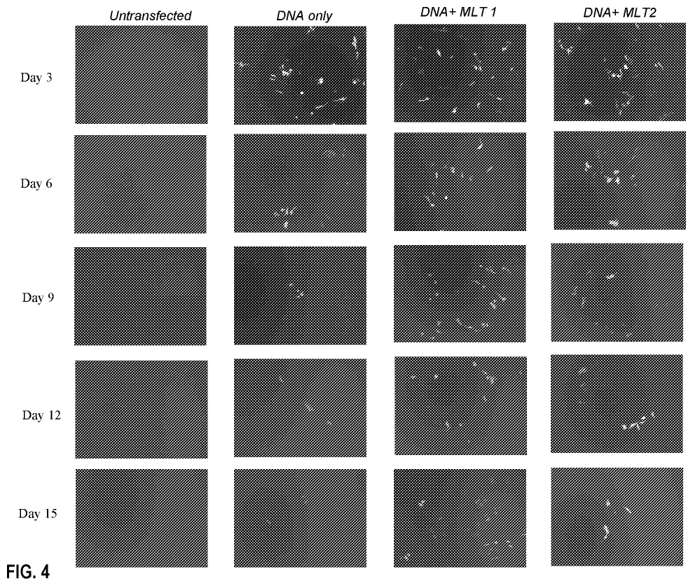

FIG. 4 shows the stable integration of donor DNA (GFP) by transposition in mouse photoreceptor cell line 661W after 4 rounds of splitting over 15 days.

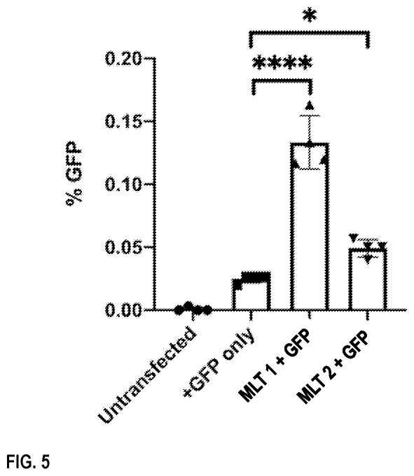

FIG. 5 illustrates results of FACS analysis of stable integration of donor DNA (GFP) by transposition in mouse photoreceptor cell line 661W on day 15.

As shown in FIG. 3, all un-transfected cells did not display any GFP expression. The use of MLT transposase 1 for a transfection resulted in GFP expression present in 661W cells after 24 hours. The same was observed for the MLT transposase 2 (FIG. 3). MAX+CAG-GFP did not express much GFP in either the MLT transposase 1 or the MLT transposase 2 transfections. L3+CAG-GFP expressed a small amount of GFP 24 hours post transfection. LTX+CAG-GFP expressed a moderate amount of GFP 24 hours post transfection. LTX had 40-50% of cells expressing GFP 24 hours post transfection.

The GFP continued to express in the transfected cells only in conditions where helper RNA (MLT transposase 1 or MLT transposase 1) were co-overexpressed with the GFP donor DNA for long time (FIG. 4). Gets were split 4 times over the period of 15 days, and donor only DNA condition lost its expression, while the donor DNA (GFP) with either MLT transposase 1 or with MLT transposase 2 continued to express GFP.

FACS analysis was carried out on day 15th for at the four conditions (FIG. 5). FACS data suggest MU transposase 1 shows more GFP expression as compared to the cells co-transfected with GFP donor DNA with the MLT transposase 2. Both MLT transposase 1 and the MLT transposase 2 showed significantly higher expression of GFP as compared to the donor DNA alone or untransfected conditions.

In sum, this data shows that, for lipofectamine, LTX (Lipofectamine with PLUS Reagent) is efficacious reagent for transposing 661W cells with CAG-GFP and either MLT transposase 1 or MLT transposase 2. Both MLT transposase 1 and MLT transposase 2 had similar GFP expression 24 hours post transfection and thus yielded stable integration of the donor DNA by transposition. For the 661W cell type, MLT transposase 1 showed more effective transposition as compared to MLT transposase 2.

### Example 7

### ARPE-19 Human Retinal Pigment Epithelial Cell Transfection with MLT Transposase

An objective of this study was to evaluate the effects of helper RNA transposase (Ts) to donor DNA transposon to two different helper RNA transposases (MLT transposase 1 and MLT transposase 2) on stable green fluorescent protein (GFP) expression in retinal cell lines using a CAG-GFP donor construct.

ARPE-19 cells were transfected with a ratio of donor transposon DNA (CAG-GFP):MLT transposase 1 and MLT transposase 2 mRNA (Donor DNA:Helper RNA) of 10 ug:5 ug. Conditions which result in cultures with relatively high numbers of GFP positive cells were kept in culture by passage for 7 to 14 days, 14 days is expected to be a sufficient period of time to allow for loss of transient expression of GFP. Cells were imaged at different time points post-transfection to monitor expression and determine which condition is allowing for GFP expression out to 14 days. Optimal transfected cultures were imaged and analyzed by flow cytometry to determine the percentage of cells which have retained OFF expression.

The following agents were used in the present study: donor DNA (>1 ug/ul, 300 ul, 1×TE buffer, endotoxin-free, sterile), helper RNA MLT transposase 1 (>500 ng/ul, 100 ul, nuclease-free water, sterile), helper RNA MLT transposase 2 (>500 ng/ul, 100 ul, nuclease-free water, sterile). Table 2 shows reagents used in the present study.

TABLE 2
Reagents used in the present study.

| Reagents | Supplier & Catalog Number |
|---|---|
| DNA | CAG-GFP (VB200819-1024gzm) |
| RNA | MLT transposase 1 (VB200905-1046fxw) |
| RNA | MLT transposase 2 (VB200905-1047pvx) |
| Lipofectamine | ThermoFisher (Invitrogen ™) Catalog |
| 3000 (L3) | Number L3000-001 |
| Lipofectamine | ThermoFisher (Invitrogen ™) Catalog |
| LTX & PLUS | Number A12621 |
| reagent (LTX) | |
| Lipofectamine | ThermoFisher (Invitrogen ™) Catalog |
| Messenger MAX | Number LMRNA001 |
| (MAX) | |

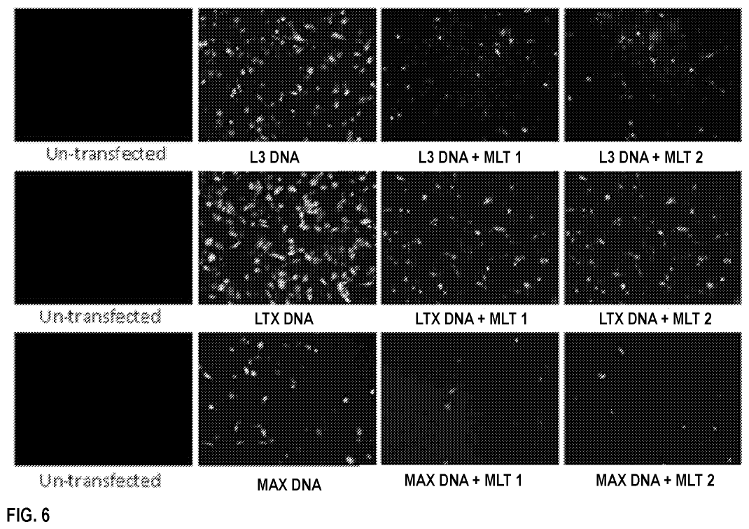

FIG. 6 shows expression of GFP in ARPE-19 cells at 24 hours post transfection. For this experiment, ARPE-19 cells were seeded in 24 well plate. 24 hours later, the cells were transfected with three different transfection systems: L3 (Lipofectamine 3000, ThermoFisher Catalog #L3000-001), LTX (Lipofectamine LTX & PLUS, ThermoFisher Catalog #A12621), and MAX (Lipofectamine Messenger MAX, ThermoFisher Catalog #LMRNA001). Then, 24 hours post-transfection, the cells were imaged for GFP.

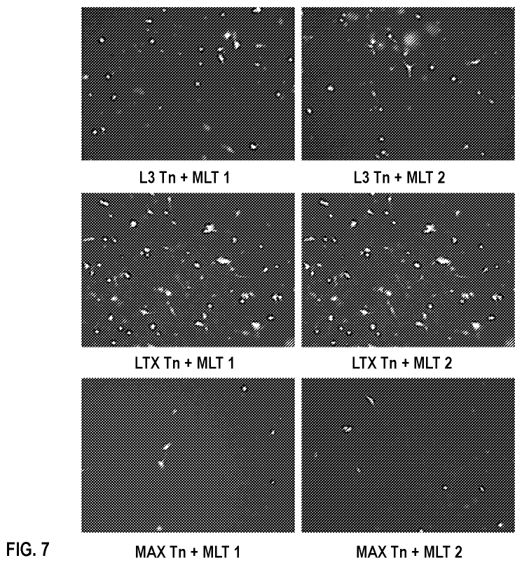

FIG. 7 shows higher resolution images of MLT transposase 1 and MLT transposase 2, visible GFP expression at 24 hours post transfection.

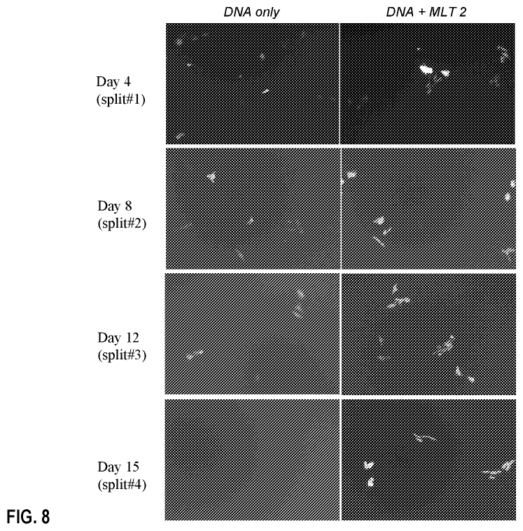

FIG. 8 shows stable integration of donor DNA (GFP) in photoreceptor cell line ARPE19 with MLT transposase 2.

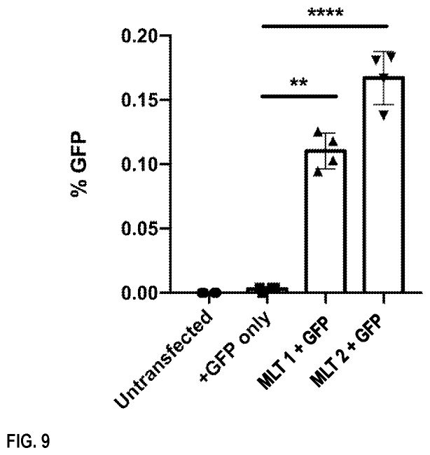

FIG. 9 illustrates that the FACS analysis shows stable GFP expression from ARPE19 cell lines after 4 generations of cell divisions.

As shown in the results of the present study, all un-transfected cells did not display any GFP expression, which can be seen in FIG. 6. L3 and only CAG-GFP expressed GFP presence after 24 hours post transfection. LTX and only CAG-GFP expressed the most GFP presence after 24 hours post transfection. MAX and CAG-GFP displayed moderate GFP expression 24 hours post transfection as well. When MLT transposase 1 was added to the lipofection reagent and CAG-GFP, there was still GFP expression present in cells after 24 hours, but it was not as much as the lipofection reagent and only CAG-GFP. The same was true for MLT transposase 2 (see FIG. 6), MLT transposase 1 and MLT transposase 2 were similar in their GFP expression efficiency, which can be seen in FIG. 7, with a side-by-side comparison of lipofection reagent+DNA with both MLT transposase 1 (left column) and MLT transposase 2 (right column).

Donor DNA, GFP was found to be integrated stably in the ARPE19 cell line, only when it was co-overexpressed with the helper either MLT transposase 1 or MLT transposase 2. The expression of GFP was investigated for 15 days and 4 splits in between to make sure the signals that are visible are not transient. The donor-only condition lost its GFP expression after 2nd split (see FIG. 8).

The flow cytometry analysis revealed that MLT transposase 2 was significantly more effective in stable transposition of donor (GFP) as compared to other conditions such as untransfected or donor only, MLT transposase 1 also appeared to be effective in stable integration of GFP (FIG. 9).

Lipofectamine & PLUS was an efficient lipofection reagent when using just CAG-GFP as well as using both CAG-GFP and either MUT transposase 1 or MLT transposase 2. Both MLT transposase 1 and MLT transposase 2 had similar GFP expression rates for these ARPE-19 cells: These data show that MLT transposase 1 and MLT transposase 2 both are efficient in stable transposition of donor DNA into the genome. However, MLT transposase 2 is more effective in stable integration of donor DNA in ARPE19 cell line than MLT transposase 1.

### Example 8

Mouse In Vivo Sub-Retinal LNP Dose Pharmacodynamics using Donor DNA (CAG-GFP)/MLT Transposase

An objective of this study was to analyze the levels of GFP expression in the mouse retina after sub-retinal injection of two doses (high and low) of a lipid nanoparticle (LNP) formulation comprising a nucleic acid encoding a donor DNA (CAG-GFP) and a nucleic acid encoding a helper RNA (MLT transposase 2 or MLT 2).

In the present study, GFP expression in the mouse retina was measured after sub-retinal injection of the two doses of a lipid nanoparticle formulations comprising a donor DNA (CAG-GFP) and a helper RNA (MLT transposase 2 or MLT 2), at a ratio of 2:1. The “high” dose was 500 ng/uL (333 ng donor DNA/166 ng helper RNA), and the “low” dose was 250 ng/uL (166 ng donor DNA/83 ng helper RNA).

Results of retinal GFP expression in the photoreceptor and RPE cell layers were measured by immunohistochemistry (IHC).

The left eye was injected with a donor DNA (CAG-GFP) and MLT transposase 2 (MLT with S8P/C13R mutations) co-encapsulated in a lipid nanoparticle. The right eye was injected with only the donor DNA encapsulated by a lipid nanoparticle. A goal was to demonstrate that the MLT transposase 2 can transfect ARPE-19 cells in the retina without causing cell damage.

In the present study, a DNA encoding CAG-GFP (VB200819-1024gzm) was used, and an RNA encoding the MLT transposase 2 (VB200926-1055qkq) was used. The LNP formulation had a cationic lipid, cholesterol, a phospholipid, and a PEG lipid: Table 2 includes information on the mice used in the present experiments:

TABLE 2
Description of test animals and agents administered to the animals.

| Mouse | | | | | Vol | Concentration | Dilution | Stock | Buffer |
|---|---|---|---|---|---|---|---|---|---|
| Group | Treatment | #Males | #Females | Formulation | (uL) | (ug) | of Stock | (uL) | (uL) |
| 1 | Control | 1 | 1 | Empty | 1 | | | | |
| | | | | LNP | | | | | |
| 2 | MLT | 1 | 1 | LNP | 1 | 333/166 | 1:1 | 100 | 100 |
| | (1 or 2) | | | | | | | | |
| 3 | MLT | 1 | 1 | LNP | 1 | 166/83 | 1:2 | 100 | 200 |
| | (1 or 2) | | | | | | | | |

Results

The images of mouse eyes were captured using Phoenix MICRON IV™ Retinal Imaging Microscope, fundus imaging.

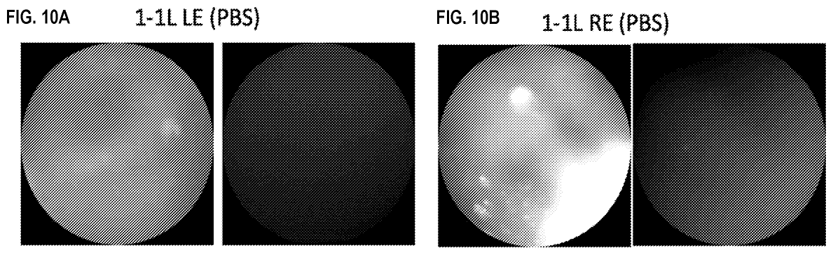

FIGS. 10A and 10B show images of mouse 1-1L left (FIG. 10A) and 1-1L right (FIG. 10B) eyes injected with PBS.

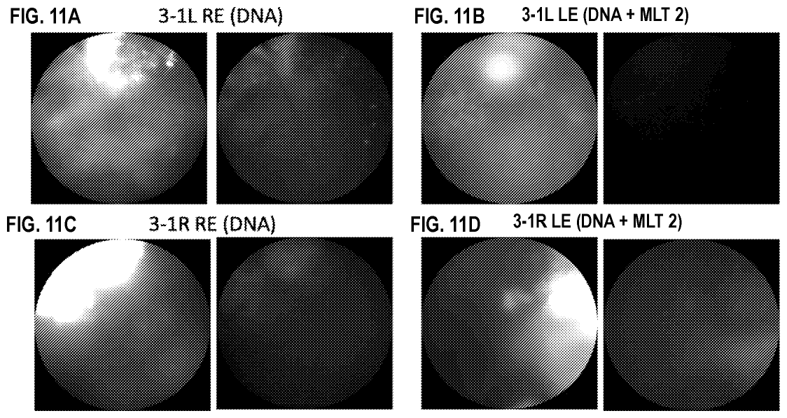

FIGS. 11A, 11B, 11C, and 11D show images of mice 3-1L and 3-1R right eyes injected with only DNA (FIG. 11A and FIG. 11C) and mice 3-1L and 3-1R left eyes injected with a donor DNA and MLT 2 (FIG. 11B and FIG. 11D).

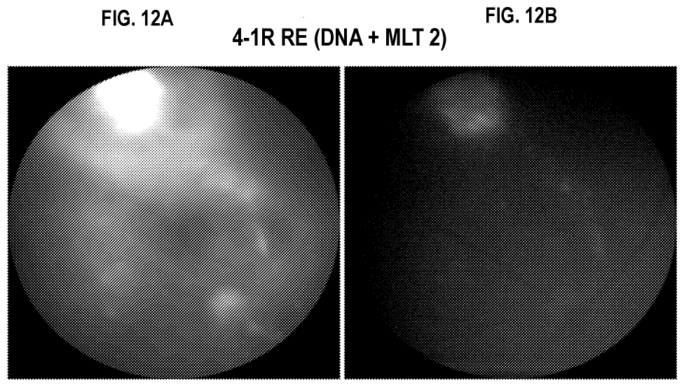

FIGS. 12A and 12B show images of mouse 4-1R's right eye injected with a donor DNA (FIG. 12A) and MLT 2 (FIG. 12B).

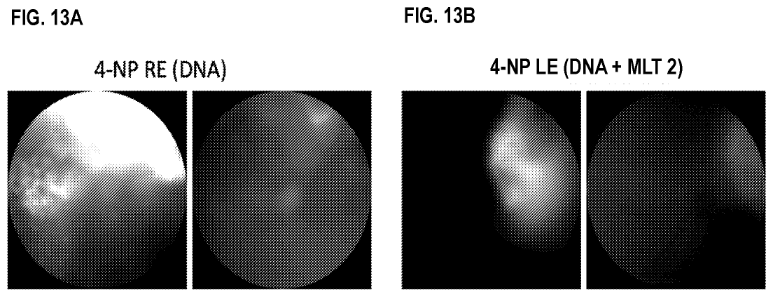

FIGS. 13A and 13B show images of mouse 4-NP right eye (FIG. 13A) injected with only a donor DNA, and left eye (FIG. 13B) injected with both the donor DNA and MLT 2.

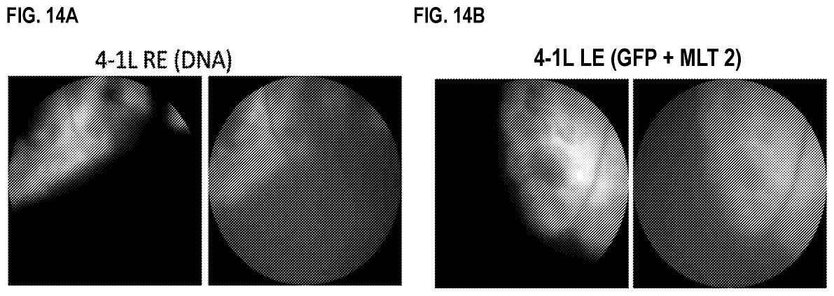

FIGS. 14A and 14B show images of mouse 4-1L right eye (FIG. 14A) injected with only a donor DNA, and left eye (FIG. 14B) injected with both the donor DNA and MLT 2.

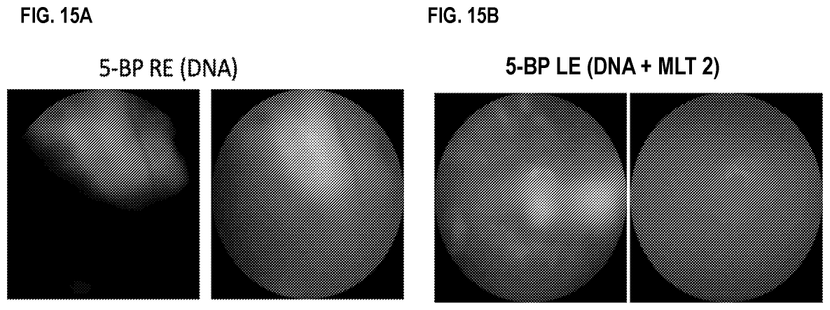

FIGS. 15A and 15B show images of mouse 5-BP right eye (FIG. 15A) injected with only a donor DNA, and left eye (FIG. 15B) injected with both the donor DNA and MLT 2.

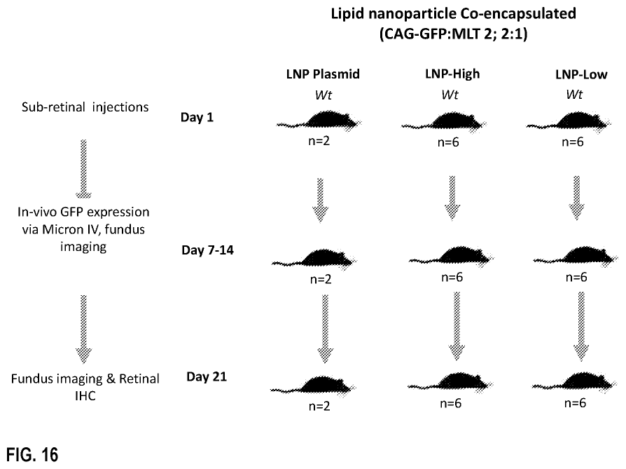

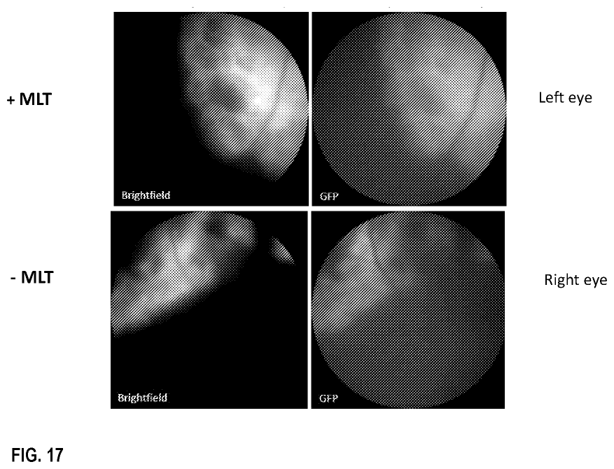

FIG. 16 illustrates a general set-up of the present study, and additionally shows that images were taken on day 21 post sub-retinal injections. FIG. 17 shows images of mouse left and right eyes (top and bottom rows, respectively), taken on day 21 day post sub-retinal injection, with (“'MLT”) or without (“−MLT”) the MLT transposase used in the transfection. In FIG. 17, the right eye is the control (the donor DNA only) and the left eye is the treated eye (the donor DNA+MLT 2 transposase).

FIGS. 10A, 10B, 11A-11D, 12A, 12B, 13A, 13B, 14A, 14B, 15A, 15B and 17 show images of the mouse eyes treated with the high dose of 500 ng/uL.

The results of this study show that the MLT transposase 2 does not negatively affect the mouse eye when injected subretinally while co-encapsulated with the donor DNA (CAG-GFP, in this example). As shown in FIGS. 14A and 14B, both eyes, 7 days post subretinal injection were not visibly damaged and exhibited GFP expression. Some surgical efficiency variation between animal to animal and also between left and right eye of a same animal were noticed.

In the present study, the MLT transposase dose that results in successful transposition of a gene from a donor DNA, was determined to be 500 ng/uL (333 ng DNA/166 ng RNA).

In conclusion, the present study shows a positive expression of a transgene (green fluorescent protein (GFP), used as a working example of a transgene) upon injection of the LNPs into the eyes sub-retinally. The expression of the transgene continued until 21 days (see FIG. 17), demonstrating feasibility of the present approach for a therapeutic use.

### Equivalents

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth and as follows in the scope of the appended claims.

Those skilled in the art will recognize, or be able to ascertain, using no more than routine experimentation, numerous equivalents to the specific embodiments described specifically herein. Such equivalents are intended to be encompassed in the scope of the following claims.

### Incorporation By Reference

All patents and publications referenced herein are hereby incorporated by reference in their entireties.

The publications discussed herein are provided solely for their disclosure prior to the filing date of the present application. Nothing herein is to be construed as an admission that the present invention is not entitled to antedate such publication by virtue of prior invention.

As used herein, all headings are simply for organization and are not intended to limit the disclosure in any manner. The content of any individual section may be equally applicable to all sections.

What is claimed is:

1. A composition comprising a gene transfer construct, comprising:
(a) a nucleic acid an ATP Binding Cassette Subfamily A Member 4 (ABC) transporter (ABCA4) protein, or a functional fragment thereof;
(b) a retina-specific promoter; and
(c) a non-viral vector comprising one or more transposase recognition sites and one or more inverted terminal repeats (ITRs) or end sequences.

2. The composition of claim 1, wherein the gene transfer construct comprises DNA or RNA.

3. The composition of claim 1 or 2, wherein the gene transfer construct is codon optimized.

4. The composition of any one of claims 1 to 3, wherein the ABCA4 protein is human ABCA4 protein, or a functional fragment thereof.

5. The composition of claim 4, wherein the nucleic acid encoding the human ABCA4 protein, or a functional fragment thereof comprises a nucleotide sequence encoding a protein having an amino acid sequence of SEQ ID NO: 1, or a variant having at least about 90%, or at least about 93%, or at least about 95%, or at least about 97%, or at least about 98% identity thereto.

6. The composition of claim 4, wherein the nucleic acid encoding the human ABCA4 protein, or a functional fragment thereof comprises a nucleotide sequence of SEQ ID NO: 2, or a variant having at least about 90%, or at least about 93%, or at least about 95%, or at least about 97%, or at least about 98% identity thereto.

7. The composition of any one of claims 1 to 6, wherein the retina-specific promoter is a human promoter.

8. The composition of any one of claims 1 to 7, wherein the retina-specific promoter is a retinal pigment epithelium (RPE) promoter, optionally selected from retinal pigment epithelium-specific 65 kDa protein (RPE65) promoter, interphotoreceptor retinoid-binding protein (IRBP) promoter, and vitelliform macular dystrophy 2 (VMD2) promoter, or a photoreceptor promoter, optionally selected from PDE, rhodopsin kinase (GRK1), CAR (cone arrestin), RP1, and L-opsin, or a functional fragment of a variant having at least about 50%, or at least about 60%, or at least about 70%, or at least about 80%, or at least about 85%, or at least about 90%, or at least about 93%, or at least about 95%, or at least about 97%, or at least about 98% identity thereto.

9. The composition of any one of claims 1 to 8, wherein the promoter is CMV enhancer, chicken beta-Actin promoter and rabbit beta-Globin splice acceptor site (CAG), optionally comprising a nucleic acid sequence of SEQ ID NO: 16, or a functional fragment of a variant having at least about 50%, or at least about 60%, or at least about 70%, or at least about 80%, or at least about 85%, or at least about 90%, or at least about 93%, or at least about 95%, or at least about 97%, or at least about 98% identity thereto.

10. The composition of claim 8, wherein the RPE promoter comprises a nucleic acid sequence of SEQ ID NO: 3, SEQ ID NO: 4, or SEQ ID NO: 5, or a functional fragment of a variant having at least about 50%, or at least about 60%, or at least about 70%, or at least about 80%, or at least about 85%, or at least about 90%, or at least about 93%, or at least about 95%, or at least about 97%, or at least about 98% identity thereto.

11. The composition of claim 8, wherein the photoreceptor promoter comprises a nucleic acid sequence of SEQ ID NO: 6, SEQ ID NO: 7, SEQ ID NO: 8, or SEQ ID NO: 9, or a functional fragment of a variant having at least about 50%, or at least about 60%, or at least about 70%, or at least about 80%, or at least about 85%, or at least about 90%, or at least about 93%, or at least about 95%, or at least about 97%, or at least about 98% identity thereto.

12. The composition of any one of claims 1 to 11, wherein the non-viral vector is a DNA plasmid.

13. The composition of claim 12, wherein the DNA plasmid comprises one or more insulator sequences that prevent or mitigate activation or inactivation of nearby genes.

14. The composition of any one of claims 1 to 13, wherein:
the ITRs or the end sequences are those of a piggyBac-like transposon, optionally comprising a TTA repetitive sequence; and/or
the ITRs or the end sequences flank the nucleic acid encoding the ABCA4 protein.

15. The composition of any one of claims 1 to 14, wherein the non-viral vector further comprising a nucleic acid construct encoding a transposase, optionally an RNA transposase plasmid.

16. The composition of any one of claims 1 to 14, further comprising a nucleic acid construct encoding a DNA transposase plasmid or an in vitro-transcribed mRNA transposase.

17. The composition of claim 15 or 16, wherein the transposase is capable of excising and/or transposing the gene from the gene transfer construct.

18. The composition(of claim 17, wherein the transposase is derived from Bombyx, Xenopus tropicalis, Trichoplusia ni, Rhinolophus ferrumequinum, Rousettus aegyptiacus, Phyllostomus discolor, Myotis myotis, Myotis lucifugus, Pteropus vampyrus, Pipistrellus kuhlii, Pan troglodytes, Molossus molossus, or Homo sapiens, and/or is an engineered version thereof and/or wherein the transposase specifically recognizes the ITRs or the end sequences.

19. The composition of any one of claims 1 to 18, wherein the gene is capable of transposition in the presence of a transposase.

20. The composition of any one of claims 1 to 19, wherein the composition is in the form of a lipid nanoparticle (LNP).

21. The composition of claim 20, comprising of one or more lipids selected from 1,2-dioleoyl-3-trimethylammonium propane (DOTAP), a cationic cholesterol derivative mixed with dimethylaminoethane-carbamoyl (DC-Chol), phosphatidyicholine (PC), triolein (glyceryl trioleate), and 1,2-distearoyl-sn-glycero-3-phosphoethanolamine-N-[carboxy(polyethylene glycol)-2000] (DSPE-PEG), 1,2-dimyristoyl-rac-glycero-3-methoxypolyethyleneglycol-2000 (DMG-PEG 2K), and 1,2 distearol -sn-glycerol-3 phosphocholine (DSPC).

22. The composition of claim 20 or 21, comprising of one or more molecules selected from polyethylenimine (PEI) and polylactic-co-glycolic acid) (PLGA), and N-Acetylgalactosamine (Gal-Nac).

23. An isolated cell comprising the composition of any one of claims 1 to 22.

24. A method for preventing or decreasing the rate of photoreceptor loss in a patient, comprising administering to a patient in need thereof a composition of any one of claims 1 to 22.

25. A method for preventing or decreasing the rate of photoreceptor loss in a patient, comprising:
(a) contacting a cell obtained from a patient or another individual with a composition of any one of claims 1 to 22; and
(b) administering the cell to a patient in need thereof.

26. The method of claim 24 or 25, wherein the method improves distance visual acuity of the patient.

27. The method of claim 24 or 25, wherein the method provides a lowering of one or more of retinaldehyde, N-retinylidene-N-retinylethanolamine (A2E) and iso-A2E relative to a level of one or more of retinaldehyde, A2E and iso-A2E without the administration, optionally greater than about a 40%, or greater than about a 50%, or greater than about a 60%, or greater than about a 70%, or greater than about a 80%, or greater than about a 90% lowering.

28. The method of claim 24 or 25, wherein the method lowers or prevents lipofuscin accumulation in the retina, optionally in the RPE and/or Bruch's membrane.

29. The method of any one of claims 24 to 28, wherein the method is performed in the absence of a steroid treatment.

30. The method of any one of claims 24 to 29, wherein the method is substantially non-immunogenic.

31. The method of any one of claims 24 to 30, wherein the prevention or decreasing of the rate of photoreceptor loss is durable.

32. The method of any one of claims 24 to 31, wherein the method requires a single administration.

33. The method of any one of claims 24 to 32, wherein the method reduces or prevents the formation of retinal pigment epithelium (RPE) debris.

34. The method of any one of claims 24 to 33, further comprising administering a nucleic acid construct encoding a transposase, optionally derived from Bombyx mori, Xenopus tropicalis, Trichoplusia ni, Rhinolophus ferrumequinum, Rousettus aegyptiacus, Phyllostomus discolor, Myotis myotis, Myotis lucifugus, Pteropus vampyrus, Pipistrellus kuhlii, Pan troglodytes, Molossus molossus, or Homo sapiens, and/or an engineered version thereof.

35. The method of any one of claims 24 to 33, further comprising contacting the cells with a nucleic acid construct encoding a transposase, optionally derived from Bombyx mori, Xenopus tropicalis, or Trichoplusia ni and/or an engineered version thereof.

36. The method of any one of claims 24 to 35, wherein the administering is intra-vitreal, or intra-retinal, or sub-vitreal, or sub-retinal.

37. The method of any one of claims 24 to 36, wherein the administering is to RPE cells and/or photoreceptors.

38. The method of any one of claims 24 to 37, wherein the administering is by injection.

39. The method of any one of claims 34 to 38, wherein the ratio of nucleic acid encoding the ABCA4 protein, or a functional fragment thereof to nucleic acid construct encoding the transposase is about 5:1, or about 4:1, or about 3:1, or about 2:1, or about 1:1, or about 1:2, or about 1:3, or about 1:4, or about 1:5.

40. The method of any one of claims 34 to 39, wherein the ratio of nucleic acid encoding the ABCA4 protein, or a functional fragment thereof to nucleic acid construct encoding the transposase is about 2:1.

41. A method for treating and/or mitigating Inherited Macular Degeneration (IMD), comprising administering to a patient in need thereof a composition of any one of claims 1-22.

42. A method for treating and/or mitigating Inherited Macular Degeneration (IMD), comprising:
(a) contacting a cell obtained from a patient or another individual with a composition of any one of claims 1 to 22; and
(b) administering the cell to a patient in need thereof.

43. The method of claim 41 or 42, wherein the IPM is STGD, and wherein the STGD disease optionally is STGD Type 1 (STGD1).

44. The method of any one of claims 41 to 43, wherein the IMD is characterized by one or more mutations in one or more of ABCA4, ELOVL4, PROM1, BEST1 and PRPH2, the ABCA4 mutations optionally being autosomal recessive mutations.

45. The method of any one of claims 41 to 44, wherein the method provides improved distance visual acuity and/or decreased the rate of photoreceptor loss as compared to a lack of treatment.

46. The method of any one of claims 41 to 45, wherein the method results in improvement of best corrected visual acuity (BCVA) to greater than about 20/200:

47. The method of any one of claims 41 to 45, wherein the method results in improvement of retinal or foveal morphology, as measured by fundus autofluorescence (FAF) or Spectral Domain-Optical Coherence Tomography (SD-OCT).

48. The method of any one of claims 41 to 47, wherein the method results in reduction or prevention of one or more of wavy vision, blind spots, blurriness, loss of depth perception, sensitivity to glare, impaired color vision, and difficulty adapting to dim lighting (delayed dark adaptation) in the patient.

49. The method of any one of claims 41 to 48, wherein the method obviates the need for steroid treatment.

50. The method of any one of claims 41 to 49, wherein the method improves distance visual acuity of the patient.

51. The method of any one of claims 41 to 50, wherein the method is substantially non-immunogenic.

52. The method of any one of claims 41 to 51, wherein the treatment and/or mitigation is durable.

53. The method of any one of claims 41 to 52, wherein the method requires a single administration.

54. The method of any one of claims 41 to 53, wherein the method reduces or prevents the formation of retinal pigment epithelium (RPE) debris.

55. The method of any one of claims 41 to 54, further comprising administering a nucleic acid construct encoding a transposase, optionally derived from Bombyx mori, Xenopus tropicalis, Trichoplusia ni, Rhinolophus ferrumequinum, Rousettus aegyptiacus, Phyllostomus discolor, Myotis myotis, Myotis lucifugus, Pteropus vampyrus, Pipistrellus kuhlii, Pan troglodytes, Molossus molossus, or Homo sapiens, and/or is engineered version thereof.

56. The method of any one of claims 41 to 55, wherein the administering is intra-vitreal or intra-retinal.

57. The method of any one of claims 41 to 56, wherein the administering is to RPE cells and/or photoreceptors.

58. The method of any one of claims 41 to 57, wherein the administering is by injection.

59. The method of any one of claims 42 to 54, further comprising contacting the cells with a nucleic acid construct encoding a transposase, optionally derived from Bombyx mori, Xenopus tropicalis, Trichoplusia ni, Rhinolophus ferrumequinum, Rousettus aegyptiacus, Phyllostomus discolor, Myotis myotis, Myotis lucifugus, Pteropus vampyrus, Pipistrellus kuhlii, Pan troglodytes, Molossus molossus, or Homo sapiens, and/or an engineered version thereof.

60. The method of any one of claims 55 to 59, wherein the ratio of the nucleic acid encoding the ABCA4 protein, or a functional fragment thereof to the nucleic acid construct encoding the transposase is about 5:1, or about 4:1, or about 3:1, or about 2;1, or about 1:1, or about 1:2, or about 1:3, or about 1:4, or about 1:5.

61. The method of any one of claims 55 to 60, wherein the ratio of the nucleic acid encoding the ABCA4, or a functional fragment thereof to the nucleic acid construct encoding the transposase is about 2:1.

62. A composition comprising a gene transfer construct, comprising:
(a) a nucleic acid encoding an ATP Binding Cassette Subfamily A Member 4 (ABC) transporter (ABCA4) protein, or a functional fragment thereof;
(b) CAG promoter; and
(c) a non-viral vector comprising one or more transposase recognition sites and one or more inverted terminal repeats (ITRs) or end sequences,
wherein the ABCA4 protein is human ABCA4, or a functional fragment thereof, that is encoded by a nucleotide sequence of SEQ ID NO: 2, or a variant having at least about 95% identity thereto.

63. A method for treating and/or mitigating Inherited Macular Degeneration (IMD), comprising:
(a) contacting a cell obtained from a patient or another individual with a composition of claim 62;
(b) contacting the cell with a nucleic acid construct encoding a transposase that is derived from Bombyx mori, Xenopus tropicalis, Trichoplusia ni, Rhinolophus ferrumequinum, Rousettus aegyptiacus, Phyllostomus discolor, Myotis myotis, Myotis lucifugus, Pteropus vampyrus, Pipistrellus kuhlii, Pan troglodytes, Molossus molossus, or Homo sapiens, and/or an engineered version thereof, wherein the ratio of ABCA4, or a functional fragment thereof to transposase is about 2:1; and
(c) administering the cell to a patient in need thereof.
